(19) United States
(12) Patent Application Publication
(10) Pub. No.: US 2010/0151109 A1
(43) Pub. Date: Jun. 17, 2010

(54) MODULATION OF PLANT PROTEIN LEVELS

(76) Inventors: Amr Saad Ragab, Blacksburg, VA (US); Steven Craig Bobzin, Malibu, CA (US); Daniel Mumenthaler, Bonita, CA (US); Joel Cruz Rarang, Granada Hills, CA (US)

Correspondence Address: FISH & RICHARDSON P.C., P.O. BOX 1022, MINNEAPOLIS, MN, 55440-1022, US

(21) Appl. No.: 12/519,106

(22) PCT Filed: Dec. 14, 2007
(86) PCT No.: PCT/US07/87638

§ 371 Date: Feb. 26, 2010

Related U.S. Application Data
(60) Provisional application No. 60/870,232, filed on Dec. 15, 2006.

(51) Int. Cl. A23L 1/212; C12N 5/10; A01H 5/00; C12N 15/29; A23K 1/14

(52) U.S. Cl. 426/630; 435/419; 800/298; 536/23.6; 426/635; 426/615

(57) ABSTRACT

Methods and materials for modulating, e.g., increasing or decreasing, protein levels in plants are disclosed. For example, nucleic acids encoding protein-modulating polypeptides are disclosed as well as methods for using such nucleic acids to transform plant cells. Also disclosed are plants having increased protein levels and plant products produced from plants having increased protein levels.

### CROSS-REFERENCE TO RELATED APPLICATION

This application claims priority to U.S. Patent Application No. 60/870,232, filed on Dec. 15, 2006, and entitled “MODULATION OF PLANT PROTEIN LEVELS,” the entire contents of which are incorporated herein by reference.

### TECHNICAL FIELD

This document relates to methods and materials involved in modulating (e.g., increasing or decreasing) protein levels in plants. For example, this document provides plants having increased protein levels as well as materials and methods for making plants and plant products having increased protein levels.

### BACKGROUND

Protein is an important nutrient required for growth, maintenance, and repair of tissues. The building blocks of proteins are 20 amino acids that may be consumed from both plant and animal sources. Most microorganisms such as E. coli can synthesize the entire set of 20 amino acids, whereas human beings cannot make nine of them. The amino acids that must be supplied in the diet are called essential amino acids, whereas those that can be synthesized endogenously are termed nonessential amino acids. These designations refer to the needs of an organism under a particular set of conditions. For example, enough arginine is synthesized by the urea cycle to meet the needs of an adult, but perhaps not those of a growing child. A deficiency of even one amino acid results in a negative nitrogen balance. In this state, more protein is degraded than is synthesized, and so more nitrogen is excreted than is ingested.

According to U.S. government standards, the Recommended Daily Allowance (RDA) of protein is 0.8 gram per kilogram of ideal body weight for the adult human. The biological value of a dietary protein is determined by the amount and proportion of essential amino acids it provides. If the protein in a food supplies all of the essential amino acids, it is called a complete protein. If the protein in a food does not supply all of the essential amino acids, it is designated as an incomplete protein. Meat and other animal products are sources of complete proteins. However, a diet high in meat can lead to high cholesterol or other diseases, such as gout. Some plant sources of protein are considered to be partially complete because, although consumed alone they may not meet the requirements for essential amino acids, they can be combined to provide amounts and proportions of essential amino acids equivalent to those in proteins from animal sources. Soy protein is an exception because it is a complete protein. Soy protein products can be good substitutes for animal products because soybeans contain all of the amino acids essential to human nutrition and they have less fat, especially saturated fat, than animal-based foods. The U.S. Food and Drug Administration (FDA) determined that diets including four daily soy servings can reduce levels of low-density lipoproteins (LDLs), the cholesterol that builds up in blood vessels, by as much as 10 percent (Henkel, FDA Consumer, 34:3 (2000); fda.gov/fdac/features/2000/300_soy.html). FDA allows a health claim on food labels stating that a daily diet containing 25 grams of soy protein, that is also low in saturated fat and cholesterol, may reduce the risk of heart disease (Henkel, FDA Consumer, 34:3 (2000); fda.gov/fdac/features/2000/300_soy.html).

There is a need for methods of increasing protein production in plants, which provide healthier and more economical sources of protein than animal products.

### SUMMARY

This document provides methods and materials related to plants having modulated (e.g., increased or decreased) levels of protein. For example, this document provides transgenic plants and plant cells having increased levels of protein, nucleic acids used to generate transgenic plants and plant cells having increased levels of protein, and methods for making plants and plant cells having increased levels of protein. Such plants and plant cells can be grown to produce, for example, seeds having increased protein content. Seeds having increased protein levels may be useful to produce foodstuffs and animal feed having increased protein content, which may benefit both food producers and consumers.

In one aspect, a method of modulating the level of protein in a plant is provided. The method comprises introducing into a plant cell an exogenous nucleic acid comprising a nucleotide sequence encoding a polypeptide having 80 percent or greater sequence identity to an amino acid sequence selected from the group consisting of SEQ ID NOs:96-98, SEQ ID NO:100, SEQ ID NOs:102-103, SEQ ID NOs:106-110, SEQ ID NO:112, SEQ ID NO:114, SEQ ID NO:116, SEQ ID NOs:118-119, SEQ ID NO:122, SEQ ID NOs:125-126, SEQ ID NO:128, SEQ ID NO:131, SEQ ID NOs:133-134, SEQ ID NO:136, SEQ ID NO:138, SEQ ID NOs:140-141, SEQ ID NOs:143-146, SEQ ID NO:148, SEQ ID NO:150, SEQ ID NO:152, SEQ ID NO:154, SEQ ID NOs:156-157, SEQ ID NO:159, SEQ ID NO:161, SEQ ID NO:163, SEQ ID NO:165, SEQ ID NOs:167-175, SEQ ID NO:177, SEQ ID NOs:179-182, SEQ ID NO:184, SEQ ID NO:186, SEQ ID NO:186, SEQ ID NOs:188-189, SEQ ID NO:191, SEQ ID NOs:193-205, SEQ ID NOs:207-210, SEQ ID NOs:212-220, SEQ ID NO:222, SEQ ID NO:224, SEQ ID NO:226, SEQ ID NO:228, SEQ ID NO:230, SEQ ID NO:232, SEQ ID NO:234, SEQ ID NO:236, SEQ ID NOs:238-239, SEQ ID NO:241, and SEQ ID NOs:243-247, where a tissue of a plant produced from the plant cell has a difference in the level of protein as compared to the corresponding level in tissue of a control plant that does not comprise the nucleic acid.

In another aspect, a method of modulating the level of protein in a plant is provided. The method comprises introducing into a plant cell an exogenous nucleic acid comprising a nucleotide sequence encoding a polypeptide having 80 percent or greater sequence identity to an amino acid sequence selected from the group consisting of SEQ ID NOs:96-98, SEQ ID NO:100, SEQ ID NO:102, SEQ ID NO:106, SEQ ID NO:108, SEQ ID NO:110, SEQ ID NO:112, SEQ ID NO:114, SEQ ID NO:116, SEQ ID NOs:118-119, SEQ ID NO:122, SEQ ID NO:125, SEQ ID NO:128, SEQ ID NO:131, SEQ ID NOs:133-134, SEQ ID NO:136, SEQ ID NO:138, SEQ ID NO:140, SEQ ID NO:143, SEQ ID NO:148, SEQ ID NO:150, SEQ ID NO:152, SEQ ID NO:154, SEQ ID NO:156, SEQ ID NO:159, SEQ ID NO:161, SEQ ID NO:163, SEQ ID NO:165, SEQ ID NO:167, SEQ ID NO:177, SEQ ID NO:179, SEQ ID NO:181, SEQ ID NO:184, SEQ ID NO:186, SEQ ID NO:188, SEQ ID NO:191, SEQ ID NO:193, SEQ ID NO:207, SEQ ID NO:212, SEQ ID NO:222, SEQ ID NO:224, SEQ ID NO:226, SEQ ID NO:228, SEQ ID NO:230, SEQ ID NO:232, SEQ ID NO:234, SEQ ID NO:236, SEQ ID NO:238, SEQ ID NO:241, and SEQ ID NO:243, where a tissue of a plant produced from the plant cell has a difference in the level of protein as compared to the corresponding level in tissue of a control plant that does not comprise the nucleic acid.

In another aspect, a method of modulating the level of protein in a plant is provided. The method comprises introducing into a plant cell an exogenous nucleic acid comprising a nucleotide sequence having 80 percent or greater sequence identity to a nucleotide sequence corresponding to SEQ ID NO:115, where a tissue of a plant produced from the plant cell has a difference in the level of protein as compared to the corresponding level in tissue of a control plant that does not comprise the nucleic acid.

In another aspect, a method of modulating the level of protein in a plant is provided. The method comprises introducing into a plant cell an exogenous nucleic acid comprising a nucleotide sequence having 80 percent or greater sequence identity to a nucleotide sequence selected from the group consisting of SEQ ID NO:95, SEQ ID NO:99, SEQ ID NO:101, SEQ ID NO:104, SEQ ID NO:105, SEQ ID NO:111, SEQ ID NO:113, SEQ ID NO:115, SEQ ID NO:117, SEQ ID NO:120, SEQ ID NO:121, SEQ ID NO:123, SEQ ID NO:124, SEQ ID NO:127, SEQ ID NO:129, SEQ ID NO:130, SEQ ID NO:132, SEQ ID NO:135, SEQ ID NO:137, SEQ ID NO:139, SEQ ID NO:142, SEQ ID NO:147, SEQ ID NO:149, SEQ ID NO:151, SEQ ID NO:153, SEQ ID NO:155, SEQ ID NO:158, SEQ ID NO:160, SEQ ID NO:162, SEQ ID NO:164, SEQ ID NO:166, SEQ ID NO:176, SEQ ID NO:178, SEQ ID NO:183, SEQ ID NO:187, SEQ ID NO:190, SEQ ID NO:192, SEQ ID NO:206, SEQ ID NO:211, SEQ ID NO:221, SEQ ID NO:223, SEQ ID NO:225, SEQ ID NO:227, SEQ ID NO:229, SEQ ID NO:231, SEQ ID NO:233, SEQ ID NO:235, SEQ ID NO:237, SEQ ID NO:240, SEQ ID NO:242, and SEQ ID NO:248, where a tissue of a plant produced from the plant cell has a difference in the level of protein as compared to the corresponding level in tissue of a control plant that does not comprise the nucleic acid.

The sequence identity can be 85 percent or greater, 90 percent or greater, or 95 percent or greater. The nucleotide sequence can encode a polypeptide comprising an amino acid sequence corresponding to SEQ ID NO:96. The nucleotide sequence can encode a polypeptide comprising an amino acid sequence corresponding to SEQ ID NO:102. The nucleotide sequence can encode a polypeptide comprising an amino acid sequence corresponding to SEQ ID NO:112. The nucleotide sequence can encode a polypeptide comprising an amino acid sequence corresponding to SEQ ID NO:114. The nucleotide sequence can encode a polypeptide comprising an amino acid sequence corresponding to SEQ ID NO:116. The nucleotide sequence can encode a polypeptide comprising an amino acid sequence corresponding to SEQ ID NO:118. The nucleotide sequence can encode a polypeptide comprising an amino acid sequence corresponding to SEQ ID NO:128. The nucleotide sequence can encode a polypeptide comprising an amino acid sequence corresponding to SEQ ID NO:181. The nucleic acid can comprise a nucleotide sequence corresponding to SEQ ID NO:115. The introducing step can comprise introducing the nucleic acid into a plurality of plant cells. The method can further comprise the step of producing a plurality of plants from the plant cells. The method can further comprise the step of selecting one or more plants from the plurality of plants that have the difference in the level of protein. The method can further comprise the step of producing a plant from the plant cell.

The difference can be an increase in the level of protein. The exogenous nucleic acid can be operably linked to a regulatory region. The regulatory region can be a promoter. The promoter can be a tissue-preferential, broadly expressing, or inducible promoter. The promoter can be a maturing endosperm promoter. The plant can be a dicot. The plant can be a member of the genus Arachis, Brassica, Carthamus, Glycine, Gossypium, Helianthus, Lactuca, Linum, Lycopersicon, Medicago, Olea, Pisum, Solanum, Trifolium, or Vitis. The plant can be a monocot. The plant can be a member of the genus Avena, Elaeis, Hordeum, Musa, Oryza, Phleum, Secale, Sorghum, Triticosecale, Triticum, or Zea. The nucleotide sequence can encode a polypeptide comprising an amino acid sequence corresponding to SEQ ID NO:96, and the plant can be a member of the genus Oryza. The nucleotide sequence can encode a polypeptide comprising an amino acid sequence corresponding to SEQ ID NO:112, and the plant can be a member of the genus Oryza. The tissue can be seed tissue.

A method of producing a plant tissue is also provided. The method comprises growing a plant cell comprising an exogenous nucleic acid comprising a nucleotide sequence encoding a polypeptide having 80 percent or greater sequence identity to an amino acid sequence selected from the group consisting of SEQ ID NOs:96-98, SEQ ID NO:100, SEQ ID NOs:102-103, SEQ ID NOs:106-110, SEQ ID NO:112, SEQ ID NO:114, SEQ ID NO:116, SEQ ID NOs:118-119, SEQ ID NO:122, SEQ ID NOs:125-126, SEQ ID NO:128, SEQ ID NO:131, SEQ ID NOs:133-134, SEQ ID NO:136, SEQ ID NO:138, SEQ ID NOs:140-141, SEQ ID NOs:143-146, SEQ ID NO:148, SEQ ID NO:150, SEQ ID NO:152, SEQ ID NO:154, SEQ ID NOs:156-157, SEQ ID NO:159, SEQ ID NO:161, SEQ ID NO:163, SEQ ID NO:165, SEQ ID NOs:167-175, SEQ ID NO:177, SEQ ID NOs:179-182, SEQ ID NO:184, SEQ ID NO:186, SEQ ID NO:186, SEQ ID NOs:188-189, SEQ ID NO:191, SEQ ID NOs:193-205, SEQ ID NOs:207-210, SEQ ID NOs:212-220, SEQ ID NO:222, SEQ ID NO:224, SEQ ID NO:226, SEQ ID NO:228, SEQ ID NO:230, SEQ ID NO:232, SEQ ID NO:234, SEQ ID NO:236, SEQ ID NOs:238-239, SEQ ID NO:241, and SEQ ID NOs:243-247, where the tissue has a difference in the level of protein as compared to the corresponding level in tissue of a control plant that does not comprise the nucleic acid.

In another aspect, a method of producing a plant tissue is provided. The method comprises growing a plant cell comprising an exogenous nucleic acid comprising a nucleotide sequence encoding a polypeptide having 80 percent or greater sequence identity to an amino acid sequence selected from the group consisting of SEQ ID NOs:96-98, SEQ ID NO:100, SEQ ID NO:102, SEQ ID NO:106, SEQ ID NO:108, SEQ ID NO:110, SEQ ID NO:112, SEQ ID NO:114, SEQ ID NO:116, SEQ ID NOs:118-119, SEQ ID NO:122, SEQ ID NO:125, SEQ ID NO:128, SEQ ID NO:131, SEQ ID NOs:133-134, SEQ ID NO:136, SEQ ID NO:138, SEQ ID NO:140, SEQ ID NO:143, SEQ ID NO:148, SEQ ID NO:150, SEQ ID NO:152, SEQ ID NO:154, SEQ ID NO:156, SEQ ID NO:159, SEQ ID NO:161, SEQ ID NO:163, SEQ ID NO:165, SEQ ID NO:167, SEQ ID NO:177, SEQ ID NO:179, SEQ ID NO:181, SEQ ID NO:184, SEQ ID NO:186, SEQ ID NO:188, SEQ ID NO:191, SEQ ID NO:193, SEQ ID NO:207, SEQ ID NO:212, SEQ ID NO:222, SEQ ID NO:224, SEQ ID NO:226, SEQ ID NO:228, SEQ ID NO:230, SEQ ID NO:232, SEQ ID NO:234, SEQ ID NO:236, SEQ ID NO:238, SEQ ID NO:241, and SEQ ID NO:243, where the tissue has a difference in the level of protein as compared to the corresponding level in tissue of a control plant that does not comprise the nucleic acid.

In another aspect, a method of producing a plant tissue is provided. The method comprises growing a plant cell comprising an exogenous nucleic acid comprising a nucleotide sequence having 80 percent or greater sequence identity to a nucleotide sequence corresponding to SEQ ID NO:115, where the tissue has a difference in the level of protein as compared to the corresponding level in tissue of a control plant that does not comprise the nucleic acid.

In another aspect, a method of producing a plant tissue is provided. The method comprises growing a plant cell comprising an exogenous nucleic acid comprising a nucleotide sequence having 80 percent or greater sequence identity to a nucleotide sequence selected from the group consisting of SEQ ID NO:95, SEQ ID NO:99, SEQ ID NO:101, SEQ ID NO:104, SEQ ID NO:105, SEQ ID NO:111, SEQ ID NO:113, SEQ ID NO:115, SEQ ID NO:117, SEQ ID NO:120, SEQ ID NO:121, SEQ ID NO:123, SEQ ID NO:124, SEQ ID NO:127, SEQ ID NO:129, SEQ ID NO:130, SEQ ID NO:132, SEQ ID NO:135, SEQ ID NO:137, SEQ ID NO:139, SEQ ID NO:142, SEQ ID NO:147, SEQ ID NO:149, SEQ ID NO:151, SEQ ID NO:153, SEQ ID NO:155, SEQ ID NO:158, SEQ ID NO:160, SEQ ID NO:162, SEQ ID NO:164, SEQ ID NO:166, SEQ ID NO:176, SEQ ID NO:178, SEQ ID NO:183, SEQ ID NO:187, SEQ ID NO:190, SEQ ID NO:192, SEQ ID NO:206, SEQ ID NO:211, SEQ ID NO:221, SEQ ID NO:223, SEQ ID NO:225, SEQ ID NO:227, SEQ ID NO:229, SEQ ID NO:231, SEQ ID NO:233, SEQ ID NO:235, SEQ ID NO:237, SEQ ID NO:240, SEQ ID NO:242, and SEQ ID NO:248, where the tissue has a difference in the level of protein as compared to the corresponding level in tissue of a control plant that does not comprise the nucleic acid.

The sequence identity can be 85 percent or greater, 90 percent or greater, or 95 percent or greater. The nucleotide sequence can encode a polypeptide comprising an amino acid sequence corresponding to SEQ ID NO:96. The nucleotide sequence can encode a polypeptide comprising an amino acid sequence corresponding to SEQ ID NO:102. The nucleotide sequence can encode a polypeptide comprising an amino acid sequence corresponding to SEQ ID NO:112. The nucleotide sequence can encode a polypeptide comprising an amino acid sequence corresponding to SEQ ID NO:114. The nucleotide sequence can encode a polypeptide comprising an amino acid sequence corresponding to SEQ ID NO:116. The nucleotide sequence can encode a polypeptide comprising an amino acid sequence corresponding to SEQ ID NO:118. The nucleotide sequence can encode a polypeptide comprising an amino acid sequence corresponding to SEQ ID NO:128. The nucleotide sequence can encode a polypeptide comprising an amino acid sequence corresponding to SEQ ID NO:181. The exogenous nucleic acid can comprise a nucleotide sequence corresponding to SEQ ID NO:115.

The difference can be an increase in the level of protein. The exogenous nucleic acid can be operably linked to a regulatory region. The regulatory region can be a promoter. The promoter can be a tissue-preferential, broadly expressing, or inducible promoter. The promoter can be a maturing endosperm promoter. The plant tissue can be dicotyledonous. The plant tissue can be a member of the genus Arachis, Brassica, Carthamus, Glycine, Gossypium, Helianthus, Lactuca, Linum, Lycopersicon, Medicago, Olea, Pisum, Solanum, Trifolium, or Vitis. The plant tissue can be monocotyledonous. The plant tissue can be a member of the genus Avena, Elaeis, Hordeum, Musa, Oryza, Phleum, Secale, Sorghum, Triticosecale, Triticum, or Zea. The nucleotide sequence can encode a polypeptide comprising an amino acid sequence corresponding to SEQ ID NO:96, and the plant tissue can be a member of the genus Oryza. The nucleotide sequence can encode a polypeptide comprising an amino acid sequence corresponding to SEQ ID NO:112, and the plant tissue can be a member of the genus Oryza. The tissue can be seed tissue.

A plant cell is also provided. The plant cell comprises an exogenous nucleic acid comprising a nucleotide sequence encoding a polypeptide having 80 percent or greater sequence identity to an amino acid sequence selected from the group consisting of SEQ ID NOs:96-98, SEQ ID NO:100, SEQ ID NOs:102-103, SEQ ID NOs:106-110, SEQ ID NO:112, SEQ ID NO:114, SEQ ID NO:116, SEQ ID NOs:118-119, SEQ ID NO:122, SEQ ID NOs:125-126, SEQ ID NO:128, SEQ ID NO:131, SEQ ID NOs:133-134, SEQ ID NO:136, SEQ ID NO:138, SEQ ID NOs:140-141, SEQ ID NOs:143-146, SEQ ID NO:148, SEQ ID NO:150, SEQ ID NO:152, SEQ ID NO:154, SEQ ID NOs:156-157, SEQ ID NO:159, SEQ ID NO:161, SEQ ID NO:163, SEQ ID NO:165, SEQ ID NOs:167-175, SEQ ID NO:177, SEQ ID NOs:179-182, SEQ ID NO:184, SEQ ID NO:186, SEQ ID NO:186, SEQ ID NOs:188-189, SEQ ID NO:191, SEQ ID NOs:193-205, SEQ ID NOs:207-210, SEQ ID NOs:212-220, SEQ ID NO:222, SEQ ID NO:224, SEQ ID NO:226, SEQ ID NO:228, SEQ ID NO:230, SEQ ID NO:232, SEQ ID NO:234, SEQ ID NO:236, SEQ ID NOs:238-239, SEQ ID NO:241, and SEQ ID NOs:243-247, where a tissue of a plant produced from the plant cell has a difference in the level of protein as compared to the corresponding level in tissue of a control plant that does not comprise the nucleic acid.

In another aspect, a plant cell is provided. The plant cell comprises an exogenous nucleic acid comprising a nucleotide sequence encoding a polypeptide having 80 percent or greater sequence identity to an amino acid sequence selected from the group consisting of SEQ ID NOs:96-98, SEQ ID NO:100, SEQ ID NO:102, SEQ ID NO:106, SEQ ID NO:108, SEQ ID NO:110, SEQ ID NO:112, SEQ ID NO:114, SEQ ID NO:116, SEQ ID NOs:118-119, SEQ ID NO:122, SEQ ID NO:125, SEQ ID NO:128, SEQ ID NO:131, SEQ ID NOs:133-134, SEQ ID NO:136, SEQ ID NO:138, SEQ ID NO:140, SEQ ID NO:143, SEQ ID NO:148, SEQ ID NO:150, SEQ ID NO:152, SEQ ID NO:154, SEQ ID NO:156, SEQ ID NO:159, SEQ ID NO:161, SEQ ID NO:163, SEQ ID NO:165, SEQ ID NO:167, SEQ ID NO:177, SEQ ID NO:179, SEQ ID NO:181, SEQ ID NO:184, SEQ ID NO:186, SEQ ID NO:188, SEQ ID NO:191, SEQ ID NO:193, SEQ ID NO:207, SEQ ID NO:212, SEQ ID NO:222, SEQ ID NO:224, SEQ ID NO:226, SEQ ID NO:228, SEQ ID NO:230, SEQ ID NO:232, SEQ ID NO:234, SEQ ID NO:236, SEQ ID NO:238, SEQ ID NO:241, and SEQ ID NO:243, where a tissue of a plant produced from the plant cell has a difference in the level of protein as compared to the corresponding level in tissue of a control plant that does not comprise the nucleic acid.

In another aspect, a plant cell is provided. The plant cell comprises an exogenous nucleic acid comprising a nucleotide sequence having 80 percent or greater sequence identity to a nucleotide sequence corresponding to SEQ ID NO:115, where a tissue of a plant produced from the plant cell has a difference in the level of protein as compared to the corresponding level in tissue of a control plant that does not comprise the nucleic acid.

In another aspect, a plant cell is provided. The plant cell comprises an exogenous nucleic acid comprising a nucleotide sequence having 80 percent or greater sequence identity to a nucleotide sequence selected from the group consisting of SEQ ID NO:95, SEQ ID NO:99, SEQ ID NO:101, SEQ ID NO:104, SEQ ID NO:105, SEQ ID NO:111, SEQ ID NO:113, SEQ ID NO:115, SEQ ID NO:117, SEQ ID NO:120, SEQ ID NO:121, SEQ ID NO:123, SEQ ID NO:124, SEQ ID NO:127, SEQ ID NO:129, SEQ ID NO:130, SEQ ID NO:132, SEQ ID NO:135, SEQ ID NO:137, SEQ ID NO:139, SEQ ID NO:142, SEQ ID NO:147, SEQ ID NO:149, SEQ ID NO:151, SEQ ID NO:153, SEQ ID NO:155, SEQ ID NO:158, SEQ ID NO:160, SEQ ID NO:162, SEQ ID NO:164, SEQ ID NO:166, SEQ ID NO:176, SEQ ID NO:178, SEQ ID NO:183, SEQ ID NO:187, SEQ ID NO:190, SEQ ID NO:192, SEQ ID NO:206, SEQ ID NO:211, SEQ ID NO:221, SEQ ID NO:223, SEQ ID NO:225, SEQ ID NO:227, SEQ ID NO:229, SEQ ID NO:231, SEQ ID NO:233, SEQ ID NO:235, SEQ ID NO:237, SEQ ID NO:240, SEQ ID NO:242, and SEQ ID NO:248, where a tissue of a plant produced from the plant cell has a difference in the level of protein as compared to the corresponding level in tissue of a control plant that does not comprise the nucleic acid.

The sequence identity can be 85 percent or greater, 90 percent or greater, or 95 percent or greater. The nucleotide sequence can encode a polypeptide comprising an amino acid sequence corresponding to SEQ ID NO:96. The nucleotide sequence can encode a polypeptide comprising an amino acid sequence corresponding to SEQ ID NO:102. The nucleotide sequence can encode a polypeptide comprising an amino acid sequence corresponding to SEQ ID NO:112. The nucleotide sequence can encode a polypeptide comprising an amino acid sequence corresponding to SEQ ID NO:114. The nucleotide sequence can encode a polypeptide comprising an amino acid sequence corresponding to SEQ ID NO:116. The nucleotide sequence can encode a polypeptide comprising an amino acid sequence corresponding to SEQ ID NO:118. The nucleotide sequence can encode a polypeptide comprising an amino acid sequence corresponding to SEQ ID NO:128. The nucleotide sequence can encode a polypeptide comprising an amino acid sequence corresponding to SEQ ID NO:181. The exogenous nucleic acid can comprise a nucleotide sequence corresponding to SEQ ID NO:115.

The difference can be an increase in the level of protein. The exogenous nucleic acid can be operably linked to a regulatory region. The regulatory region can be a promoter. The promoter can be a tissue-preferential, broadly expressing, or inducible promoter. The promoter can be a maturing endosperm promoter. The plant can be a dicot. The plant can be a member of the genus Arachis, Brassica, Carthamus, Glycine, Gossypium, Helianthus, Lactuca, Linum, Lycopersicon, Medicago, Olea, Pisum, Solanum, Trifolium, or Vitis. The plant can be a monocot. The plant can be a member of the genus Avena, Elaeis, Hordeum, Musa, Oryza, Phleum, Secale, Sorghum, Triticosecale, Triticum, or Zea. The nucleotide sequence can encode a polypeptide comprising an amino acid sequence corresponding to SEQ ID NO:96, and the plant can be a member of the genus Oryza. The nucleotide sequence can encode a polypeptide comprising an amino acid sequence corresponding to SEQ ID NO:112, and the plant can be a member of the genus Oryza. The tissue can be seed tissue.

A transgenic plant is also provided. The transgenic plant comprises any of the plant cells described above. Progeny of the transgenic plant are also provided. The progeny have a difference in the level of protein as compared to the level of protein in a corresponding control plant that does not comprise the exogenous nucleic acid. Seed, vegetative tissue, and fruit from the transgenic plant are also provided. In addition, food products and feed products comprising seed, vegetative tissue, and/or fruit from the transgenic plant are provided. Protein from the transgenic plant, which can be a soybean plant, is also provided.

In another aspect, an isolated nucleic acid molecule is provided. The isolated nucleic acid molecule comprises a nucleotide sequence having 95% or greater sequence identity to the nucleotide sequence set forth in SEQ ID NO:105.

In another aspect, an isolated nucleic acid is provided. The isolated nucleic acid comprises a nucleotide sequence encoding a polypeptide having 80% or greater sequence identity to the amino acid sequence set forth in SEQ ID NO:106.

In another aspect, an isolated nucleic acid molecule is provided. The isolated nucleic acid molecule comprises a nucleotide sequence having 95% or greater sequence identity to the nucleotide sequence set forth in SEQ ID NO:121.

In another aspect, an isolated nucleic acid is provided. The isolated nucleic acid comprises a nucleotide sequence encoding a polypeptide having 80% or greater sequence identity to the amino acid sequence set forth in SEQ ID NO:122.

In another aspect, an isolated nucleic acid molecule is provided. The isolated nucleic acid molecule comprises a nucleotide sequence having 95% or greater sequence identity to the nucleotide sequence set forth in SEQ ID NO:124.

In another aspect, an isolated nucleic acid is provided. The isolated nucleic acid comprises a nucleotide sequence encoding a polypeptide having 80% or greater sequence identity to the amino acid sequence set forth in SEQ ID NO:125.

In another aspect, an isolated nucleic acid molecule is provided. The isolated nucleic acid molecule comprises a nucleotide sequence having 95% or greater sequence identity to the nucleotide sequence set forth in SEQ ID NO:130.

In another aspect, an isolated nucleic acid is provided. The isolated nucleic acid comprises a nucleotide sequence encoding a polypeptide having 80% or greater sequence identity to the amino acid sequence set forth in SEQ ID NO:131.

In another aspect, an isolated nucleic acid molecule is provided. The isolated nucleic acid molecule comprises a nucleotide sequence having 95% or greater sequence identity to the nucleotide sequence set forth in SEQ ID NO:132.

In another aspect, an isolated nucleic acid is provided. The isolated nucleic acid comprises a nucleotide sequence encoding a polypeptide having 80% or greater sequence identity to the amino acid sequence set forth in SEQ ID NO:133.

A method of producing a plant also is provided. The method includes growing a plant cell that includes an exogenous nucleic acid, the exogenous nucleic acid comprising a regulatory region operably linked to a nucleotide sequence encoding a polypeptide, wherein the HMM bit score of the amino acid sequence of the polypeptide is greater than about 20, the HMM based on the amino acid sequences depicted in one of FIGS. 1 to 7, and wherein the plant has a difference in protein content as compared to the corresponding protein content of a control plant that does not comprise the nucleic acid.

In yet another aspect, a method of modulating the level of protein in a plant is provided. The method includes introducing into a plant cell an exogenous nucleic acid, the exogenous nucleic acid comprising a regulatory region operably linked to a nucleotide sequence encoding a polypeptide, wherein the HMM bit score of the amino acid sequence of the polypeptide is greater than about 20, the HMM based on the amino acid sequences depicted in one of FIGS. 1 to 7, and wherein a tissue of a plant produced from the plant cell has a difference in the protein content as compared to the corresponding protein content of a control plant that does not comprise the exogenous nucleic acid.

A plant cell comprising an exogenous nucleic acid also is provided as well as a transgenic plant that comprises such a plant cell. The exogenous nucleic acid includes a regulatory region operably linked to a nucleotide sequence encoding a polypeptide, wherein the HMM bit score of the amino acid sequence of the polypeptide is greater than about 20, the HMM based on the amino acid sequences depicted in one of FIGS. 1 to 7, and wherein a tissue of a plant produced from the plant cell has a difference in protein content as compared to the corresponding protein content of a control plant that does not comprise the nucleic acid.

Unless otherwise defined, all technical and scientific terms used herein have the same meaning as commonly understood by one of ordinary skill in the art to which this invention pertains. Although methods and materials similar or equivalent to those described herein can be used to practice the invention, suitable methods and materials are described below. All publications, patent applications, patents, and other references mentioned herein are incorporated by reference in their entirety. In case of conflict, the present specification, including definitions, will control. In addition, the materials, methods, and examples are illustrative only and not intended to be limiting.

The details of one or more embodiments of the invention are set forth in the accompanying drawings and the description below. Other features, objects, and advantages of the invention will be apparent from the description and drawings, and from the claims.

### DESCRIPTION OF THE DRAWINGS

FIG. 1 is an alignment of Lead Ceres ANNOT ID 826303 (SEQ ID NO:96) with homologous and/or orthologous amino acid sequences CeresClone:1103899 (SEQ ID NO:97), CeresClone:463034 (SEQ ID NO:98), and CeresClone:1816436 (SEQ ID NO:100). In all the alignment figures shown herein, a dash in an aligned sequence represents a gap, i.e., a lack of an amino acid at that position. Identical amino acids or conserved amino acid substitutions among aligned sequences are identified by boxes. FIG. 1 and the other alignment figures provided herein were produced using MUSCLE version 3.52 based on the sequence alignments generated with ProbCon (Do et al., Genome Res., 15(2):330-40 (2005)) version 1.11.

FIGS. 2A-2B are an alignment of Lead Annot ID 571199 (SEQ ID NO:102) with homologous and/or orthologous amino acid sequences CeresAnnot:1469254 (SEQ ID NO:106), gi|92895842 (SEQ ID NO:107), CeresClone:1121764 (SEQ ID NO:108), gi|33146851 (SEQ ID NO:109), and CeresClone:278526 (SEQ ID NO:110).

FIG. 3 is an alignment of Lead Ceres ANNOT ID 564367 (SEQ ID NO:118) with homologous and/or orthologous amino acid sequences CeresClone:594825 (SEQ ID NO:119), CeresAnnot:1486448 (SEQ ID NO:125), and gi|115479583 (SEQ ID NO:126).

FIG. 4 is an alignment of Lead Ceres ANNOT ID 851745 (SEQ ID NO:128) with homologous and/or orthologous amino acid sequences CeresAnnot 1455259 (SEQ ID NO:131), CeresClone 1939499 (SEQ ID NO:133), and CeresClone 605517 (SEQ ID NO:134).

FIGS. 5A-5D are an alignment of Ceres CLONE ID no. 97982 (SEQ ID NO:114) with homologous and/or orthologous amino acid sequences gi|46917479 (SEQ ID NO:172), gi|75298155 (SEQ ID NO:173), gi|71608998 (SEQ ID NO:220), Ceres CLONE ID 1938642 (SEQ ID NO:222), Ceres ANNOT ID 1488622 (SEQ ID NO:224), Ceres CLONE ID 593224 (SEQ ID NO:230), Ceres ANNOT ID 6014891 (SEQ ID NO:232), Ceres CLONE ID 686770 (SEQ ID NO:236), Ceres CLONE ID 1060795 (SEQ ID NO:238), gi|152955979 (SEQ ID NO:239), Ceres CLONE ID 1913328 (SEQ ID NO:241), gi|125561861 (SEQ ID NO:244), gi|42408633 (SEQ ID NO:245), and gi|7533036 (SEQ ID NO:246).

FIGS. 6A-6C are an alignment of Ceres ANNOT ID 842015 (SEQ ID NO:112) with homologous and/or orthologous amino acid sequences Ceres ANNOT ID 1531521 (SEQ ID NO:165), gi|125538901 (SEQ ID NO:174), and gi|125581583 (SEQ ID NO:175).

FIGS. 7A-7E are an alignment of full-length Ceres CLONE ID 258034 (SEQ ID NO:181) with homologous and/or orthologous amino acid sequences gi|10720232 (SEQ ID NO:182), Ceres CLONE ID 1832498 (SEQ ID NO:184), gi|10720220 (SEQ ID NO:189), Ceres ANNOT ID 1470542 (SEQ ID NO:191), gi|15239574 (SEQ ID NO:194), gi|266742 (SEQ ID NO:200), gi|10720233 (SEQ ID NO:201), gi|21068893 (SEQ ID NO:202), gi|10720236 (SEQ ID NO:204), gi|90398977 (SEQ ID NO:205), Ceres ANNOT ID 6014695 (SEQ ID NO:207), gi|10720235 (SEQ ID NO:208), gi|58003345 (SEQ ID NO:209), gi|7330644 (SEQ ID NO:210), Ceres CLONE ID 1787867 (SEQ ID NO:212), gi|18071421 (SEQ ID NO:213), gi|116058730 (SEQ ID NO:215), gi|34915978 (SEQ ID NO:216), and gi|9587209 (SEQ ID NO:218).

### DETAILED DESCRIPTION

The invention features methods and materials related to modulating (e.g., increasing or decreasing) protein levels in plants. In some embodiments, the plants may also have modulated levels of oil. The methods can include transforming a plant cell with a nucleic acid encoding a protein-modulating polypeptide, wherein expression of the polypeptide results in a modulated level of protein. Plant cells produced using such methods can be grown to produce plants having an increased or decreased protein content. Such plants, and the seeds of such plants, may be used to produce, for example, foodstuffs and animal feed having an increased protein content and nutritional value.

Polypeptides

The term “polypeptide” as used herein refers to a compound of two or more subunit amino acids, amino acid analogs, or other peptidomimetics, regardless of post-translational modification, e.g., phosphorylation or glycosylation. The subunits may be linked by peptide bonds or other bonds such as, for example, ester or ether bonds. The term “amino acid” refers to natural and/or unnatural or synthetic amino acids, including D/L optical isomers. Full-length proteins, analogs, mutants, and fragments thereof are encompassed by this definition.

Polypeptides described herein include protein-modulating polypeptides. Protein-modulating polypeptides can be effective to modulate protein levels when expressed in a plant or plant cell. Modulation of the level of protein can be either an increase or a decrease in the level of protein relative to the corresponding level in control plants.

A protein-modulating polypeptide can contain a TLD domain, which is predicted to be characteristic of an enzyme. The TLD domain is restricted to eukaryotes, and is often found associated with Fibrinogen_C6. SEQ ID NO:96 sets forth the amino acid sequence of an Arabidopsis clone, identified herein as Ceres ANNOT ID no. 826303 (SEQ ID NO:95), that is predicted to encode a polypeptide containing a TLD domain.

A protein-modulating polypeptide can comprise the amino acid sequence set forth in SEQ ID NO:96. Alternatively, a protein-modulating polypeptide can be a homolog, ortholog, or variant of the polypeptide having the amino acid sequence set forth in SEQ ID NO:96. For example, a protein-modulating polypeptide can have an amino acid sequence with at least 47% sequence identity, e.g., 50%, 52%, 56%, 59%, 61%, 65%, 70%, 75%, 80%, 85%, 90%, 95%, 97%, 98%, or 99% sequence identity, to the amino acid sequence set forth in SEQ ID NO:96.

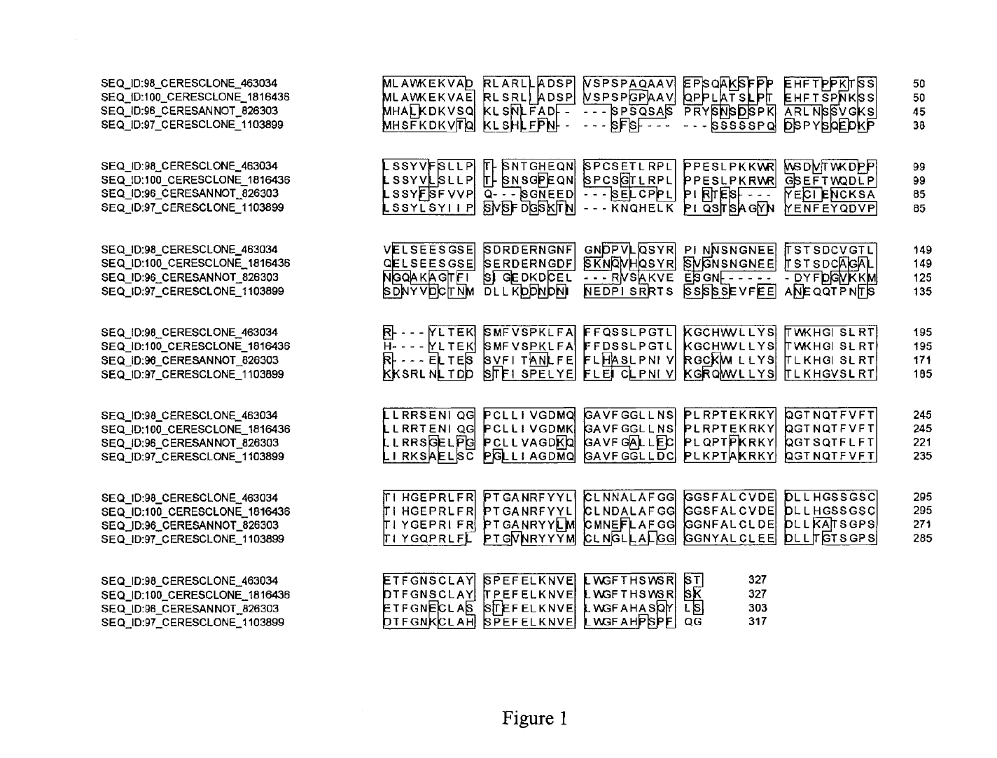

Amino acid sequences of homologs and/or orthologs of the polypeptide having the amino acid sequence set forth in SEQ ID NO:96 are provided in FIG. 1 and in the sequence listing. For example, the alignment in FIG. 1 provides the amino acid sequences of Lead Ceres ANNOT ID 826303 (SEQ ID NO:96), CeresClone:1103899 (SEQ ID NO:97), CeresClone:463034 (SEQ ID NO:98), and CeresClone:1816436 (SEQ ID NO:100). Other homologs and/or orthologs of SEQ ID NO:96 include Ceres ANNOT ID 1516248 (SEQ ID NO:148), Ceres ANNOT ID 1462954 (SEQ ID NO:150), Ceres CLONE ID 467151 (SEQ ID NO:152), Ceres ANNOT ID 6121933 (SEQ ID NO:154), Ceres CLONE ID 884270 (SEQ ID NO:156), gi|147801307 (SEQ ID NO:157), Ceres CLONE ID 1804056 (SEQ ID NO:159), Ceres CLONE ID 1795473 (SEQ ID NO:161), and Ceres CLONE ID 1811977 (SEQ ID NO:163).

In some cases, a protein-modulating polypeptide includes a polypeptide having at least 80% sequence identity, e.g., 80%, 85%, 90%, 95%, 97%, 98%, or 99% sequence identity, to an amino acid sequence corresponding to SEQ ID NO:97, SEQ ID NO:98, SEQ ID NO:100, SEQ ID NO:148, SEQ ID NO:150, SEQ ID NO:152, SEQ ID NO:154, SEQ ID NO:156, SEQ ID NO:157, SEQ ID NO:159, SEQ ID NO:161, or SEQ ID NO:163.

A protein-modulating polypeptide can contain an ATP_bind_{—}3 domain characteristic of polypeptides belonging to the PP-loop superfamily. Members of the PP-loop superfamily contain a conserved amino acid sequence motif identified in four groups of enzymes that catalyze the hydrolysis of the alpha-beta phosphate bond of ATP, namely GMP synthetases, argininosuccinate synthetases, asparagine synthetases, and ATP sulfurylases. The motif is also present in Rhodobacter capsulata AdgA, Escherichia coli NtrL, and Bacillus subtilis OutB. The observed pattern of amino acid residue conservation and predicted secondary structures suggest that this motif may be a modified version of the P-loop of nucleotide binding domains, and that it is likely to be involved in phosphate binding. More particularly, the motif appears to be a part of an ATP pyrophophatase domain. In some polypeptides, the pyrophosphatase domain is associated with amidotransferase domains (type I or type II), a putative citrulline-aspartate ligase domain, or a nitrilase/amidase domain. SEQ ID NO:112 sets forth the amino acid sequence of an Arabidopsis clone, identified herein as Ceres ANNOT ID no. 842015 (SEQ ID NO:111), that is predicted to encode a polypeptide containing an ATP_bind_{—}3 domain.

A protein-modulating polypeptide can comprise the amino acid sequence set forth in SEQ ID NO:112. Alternatively, a protein-modulating polypeptide can be a homolog, ortholog, or variant of the polypeptide having the amino acid sequence set forth in SEQ ID NO:112. For example, a protein-modulating polypeptide can have an amino acid sequence with at least 40% sequence identity, e.g., 41%, 46%, 50%, 55%, 60%, 65%, 70%, 75%, 80%, 85%, 90%, 95%, 97%, 98%, or 99% sequence identity, to the amino acid sequence set forth in SEQ ID NO:112.

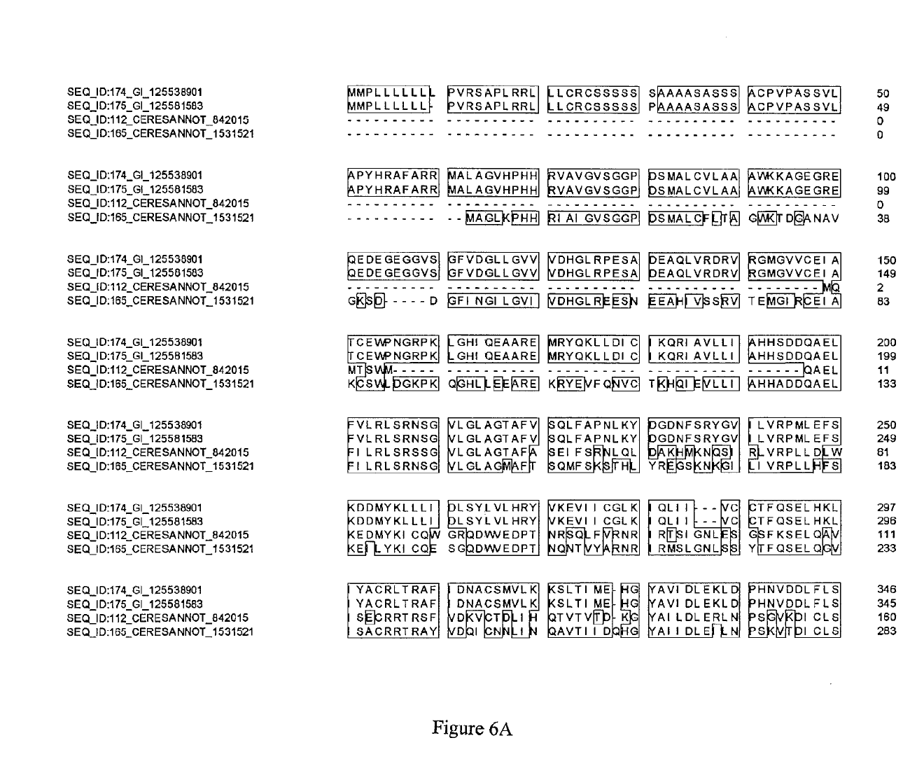

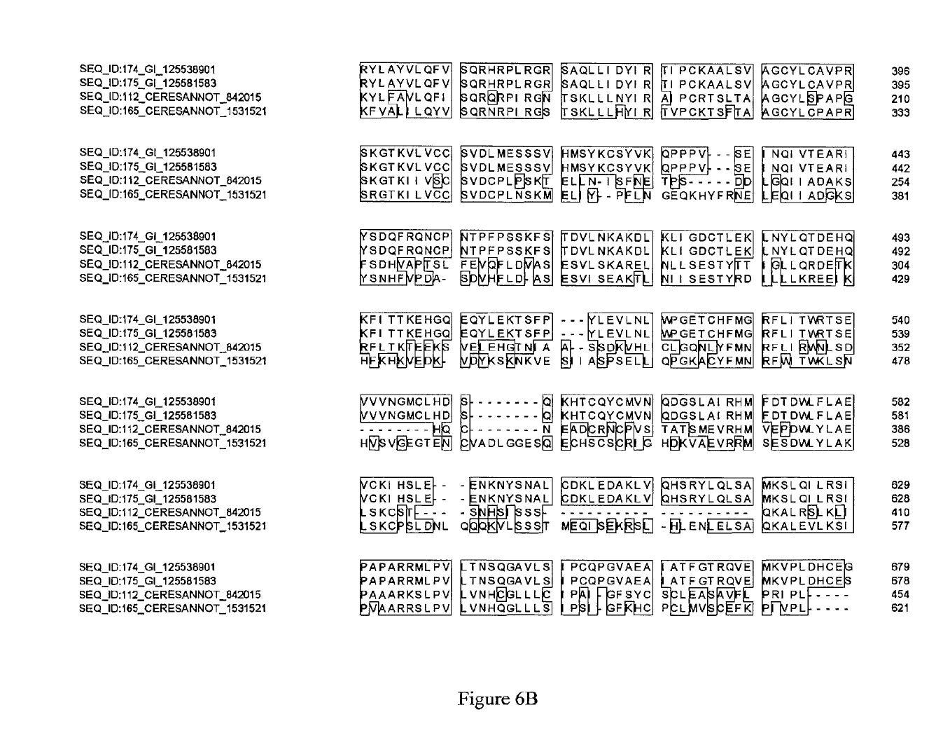

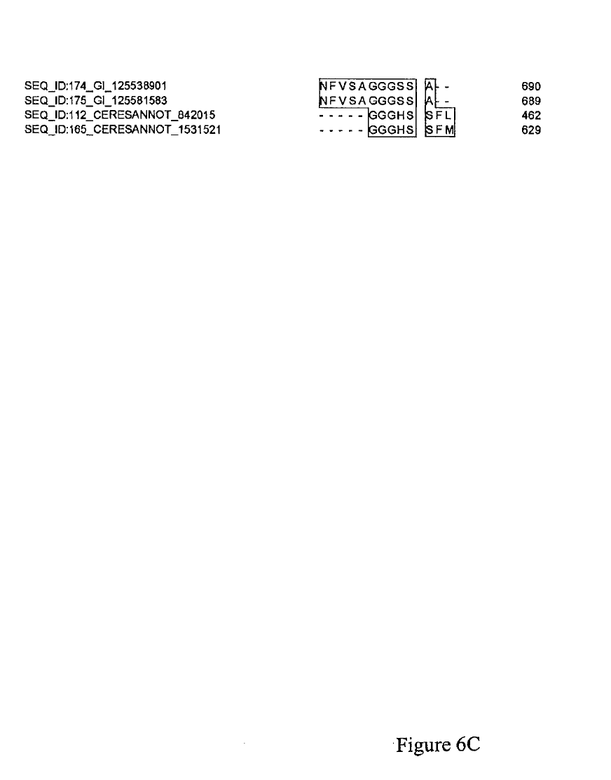

Amino acid sequences of homologs and/or orthologs of the polypeptide having the amino acid sequence set forth in SEQ ID NO:112 are provided in FIGS. 6A-6C and in the sequence listing. For example, the alignment in FIGS. 6A-6C provides the amino acid sequences of Ceres ANNOT ID no. 842015 (SEQ ID NO:112), Ceres ANNOT ID 1531521 (SEQ ID NO:165), gi|125538901 (SEQ ID NO:174), and gi|125581583 (SEQ ID NO:175). Other homologs and/or orthologs of SEQ ID NO:112 include Ceres ANNOT ID 1478015 (SEQ ID NO:167), gi|42572525 (SEQ ID NO:168), gi|42572523 (SEQ ID NO:169), gi|19071761 (SEQ ID NO:170), and gi|9294051 (SEQ ID NO:171).

In some cases, a protein-modulating polypeptide includes a polypeptide having at least 80% sequence identity, e.g., 80%, 85%, 90%, 95%, 97%, 98%, or 99% sequence identity, to an amino acid sequence corresponding to SEQ ID NO:165, SEQ ID NO:167, SEQ ID NO:168, SEQ ID NO:169, SEQ ID NO:170, SEQ ID NO:171, SEQ ID NO:174, or SEQ ID NO:175.

A protein-modulating polypeptide can contain a Carb_anhydrase domain characteristic of eukaryotic-type carbonic anhydrase polypeptides. The terms carbonic dehydratase and carbonic anhydrase are synonyms. Carbonate dehydratase polypeptides are zinc metalloenzymes that catalyze the reversible hydration of carbon dioxide. Eight enzymatic and evolutionary related forms of carbonic anhydrase are known to exist in vertebrates: three cytosolic isozymes (CA-I, CA-II and CA-III); two membrane-bound forms (CA-IV and CA-VII); a mitochondrial form (CA-V); a secreted salivary form (CA-VI); and an isozyme. SEQ ID NO:114 sets forth the amino acid sequence of an Arabidopsis clone, identified herein as Ceres CLONE ID 97982 (SEQ ID NO:113), that is predicted to encode a polypeptide containing a Carb_anhydrase domain.

A protein-modulating polypeptide can comprise the amino acid sequence set forth in SEQ ID NO:114. Alternatively, a protein-modulating polypeptide can be a homolog, ortholog, or variant of the polypeptide having the amino acid sequence set forth in SEQ ID NO:114. For example, a protein-modulating polypeptide can have an amino acid sequence with at least 40% sequence identity, e.g., 41%, 45%, 50%, 55%, 60%, 65%, 70%, 75%, 80%, 85%, 90%, 95%, 97%, 98%, or 99% sequence identity, to the amino acid sequence set forth in SEQ ID NO:114.

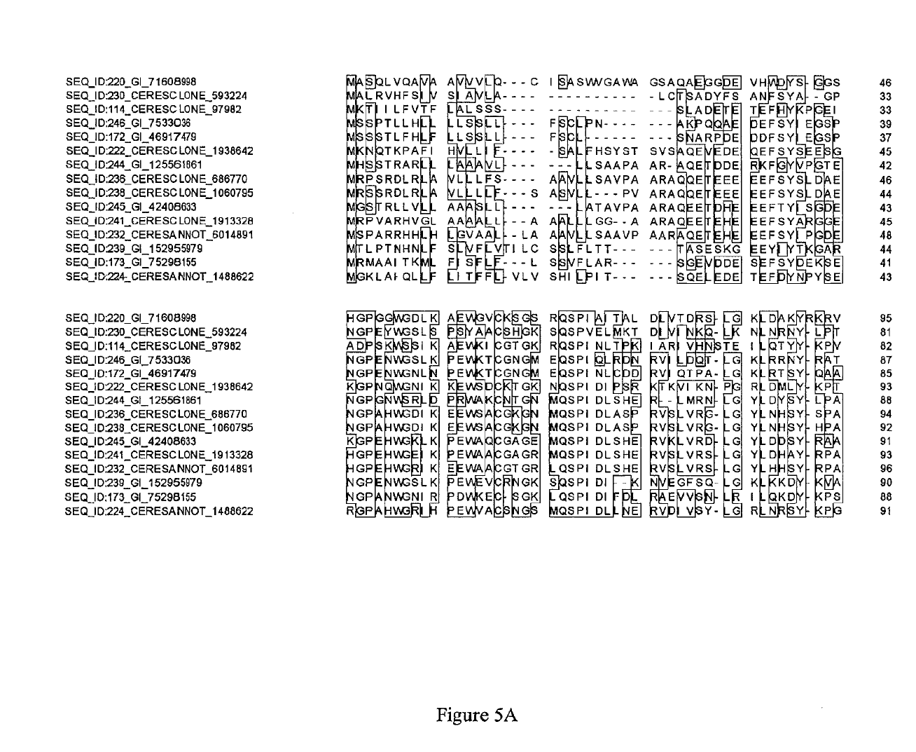

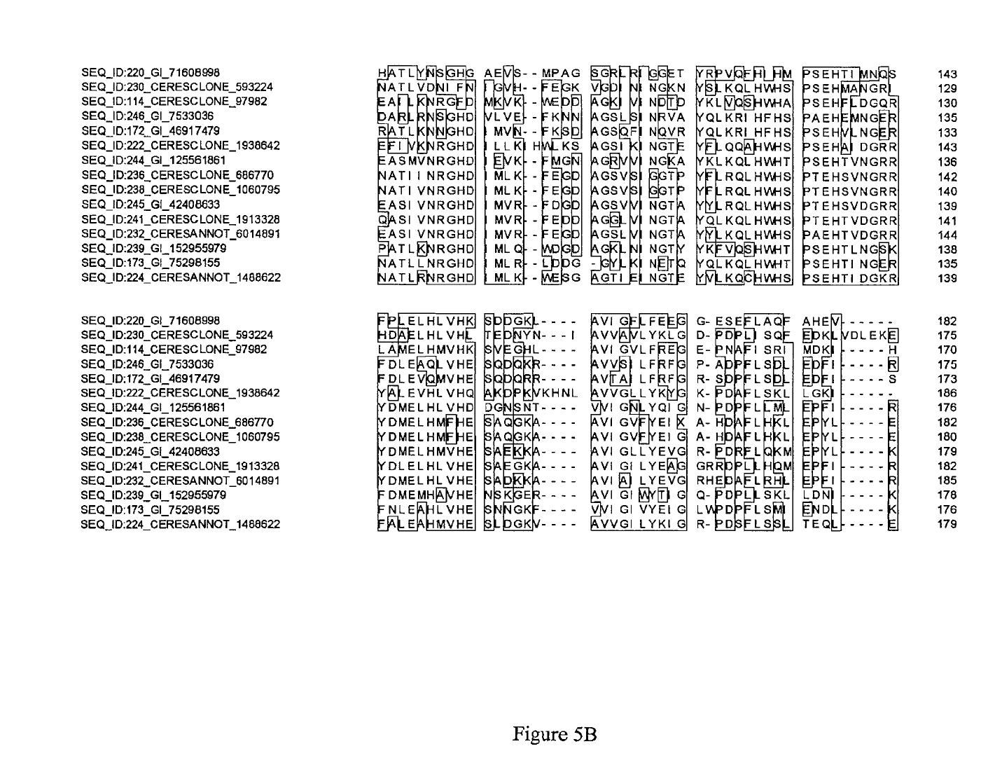

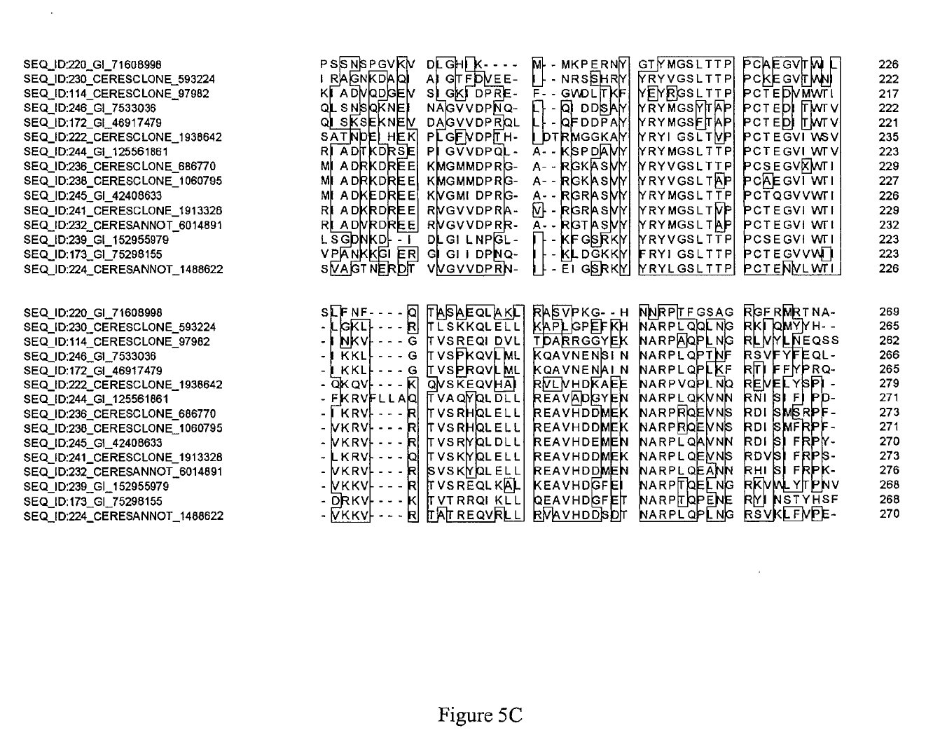

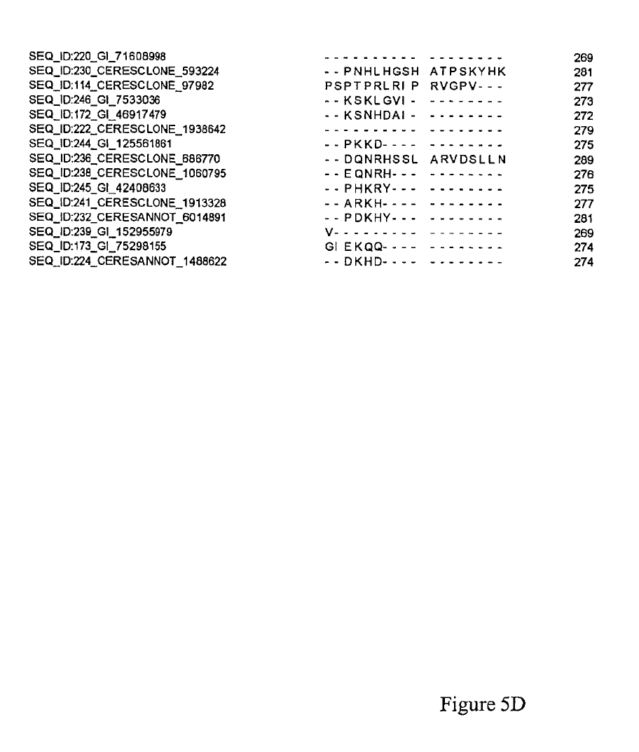

Amino acid sequences of homologs and/or orthologs of the polypeptide having the amino acid sequence set forth in SEQ ID NO:114 are provided in FIGS. 5A-5D and in the sequence listing. For example, the alignment in FIGS. 5A-5D provides the amino acid sequences of Ceres CLONE ID 97982 (SEQ ID NO:114), gi|46917479 (SEQ ID NO:172), gi|75298155 (SEQ ID NO:173), gi|71608998 (SEQ ID NO:220), Ceres CLONE ID 1938642 (SEQ ID NO:222), Ceres ANNOT ID 1488622 (SEQ ID NO:224), Ceres CLONE ID 593224 (SEQ ID NO:230), Ceres ANNOT ID 6014891 (SEQ ID NO:232), Ceres CLONE ID 686770 (SEQ ID NO:236), Ceres CLONE ID 1060795 (SEQ ID NO:238), gi|152955979 (SEQ ID NO:239), Ceres CLONE ID 1913328 (SEQ ID NO:241), gi|125561861 (SEQ ID NO:244), gi|42408633 (SEQ ID NO:245), and gi|7533036 (SEQ ID NO:246). Other homologs and/or orthologs of SEQ ID NO:114 include Ceres ANNOT ID 1462112 (SEQ ID NO:226), Ceres ANNOT ID 1515413 (SEQ ID NO:228), Ceres ANNOT ID 6014889 (SEQ ID NO:234), Ceres CLONE ID 2017488 (SEQ ID NO:243), and gi|7330291 (SEQ ID NO:247).

In some cases, a protein-modulating polypeptide includes a polypeptide having at least 80% sequence identity, e.g., 80%, 85%, 90%, 95%, 97%, 98%, or 99% sequence identity, to an amino acid sequence corresponding to SEQ ID NO:172, SEQ ID NO:173, SEQ ID NO:220, SEQ ID NO:222, SEQ ID NO:224, SEQ ID NO:226, SEQ ID NO:228, SEQ ID NO:230, SEQ ID NO:232, SEQ ID NO:234, SEQ ID NO:236, SEQ ID NO:238, SEQ ID NO:239, SEQ ID NO:241, SEQ ID NO:243, SEQ ID NO:244, SEQ ID NO:245, SEQ ID NO:246, or SEQ ID NO:247.

A protein-modulating polypeptide can contain a DnaJ domain. The DnaJ domain (J-domain) is associated with a chaperone (protein folding) system. The prokaryotic heat shock protein DnaJ interacts with the chaperone hsp70-like DnaK protein. The DnaJ protein consists of an N-terminal conserved domain (J domain) of about 70 amino acids, a glycine-rich region (G-domain) of about 30 residues, a central domain containing four repeats of a CXXCXGXG motif (CRR-domain), and a C-terminal region of 120 to 170 residues. It is thought that the J-domain of DnaJ mediates the interaction with the DnaK protein. The J-domain of DnaJ consists of four helices, the second of which has a charged surface including at least one pair of basic residues that is essential for interaction with the ATPase domain of Hsp70. The J- and CRR-domains are found in many prokaryotic and eukaryotic polypeptides, either together or separately. The T-antigens, for example, are reported to contain DnaJ domains. In yeast, J-domains have been classified into three groups; the class III proteins are functionally distinct and do not appear to act as molecular chaperones.

A protein-modulating polypeptide can contain a CSL zinc finger domain. The CSL zinc finger domain is a zinc binding motif that contains four cysteine residues that chelate zinc and is often found associated with the DnaJ domain. The CSL zinc finger domain also can be found in DPH3 and DPH4, two proteins that are involved in the biosynthesis of diphthamide, a post-translationally modified histidine residue found only in translation elongation factor 2 (eEF-2). It is conserved from archaea to humans and serves as the target for diphteria toxin and Pseudomonas exotoxin A. These two toxins catalyze the transfer of ADP-ribose to diphtamide on eEF-2, thus inactivating eEF-2, halting cellular protein synthesis, and causing cell death. See Collier (Toxicon. 2001 39(11):1793-803). SEQ ID NO:118 sets forth the amino acid sequence of an Arabidopsis clone, identified herein as Ceres ANNOT ID no. 564367 (SEQ ID NO:117), that is predicted to encode a polypeptide containing a DnaJ domain and a CSL zinc finger domain.

A protein-modulating polypeptide can comprise the amino acid sequence set forth in SEQ ID NO:118. Alternatively, a protein-modulating polypeptide can be a homolog, ortholog, or variant of the polypeptide having the amino acid sequence set forth in SEQ ID NO:118. For example, a protein-modulating polypeptide can have an amino acid sequence with at least 45% sequence identity, e.g., 46%, 50%, 55%, 60%, 65%, 70%, 75%, 80%, 85%, 90%, 95%, 97%, 98%, or 99% sequence identity, to the amino acid sequence set forth in SEQ ID NO:118.

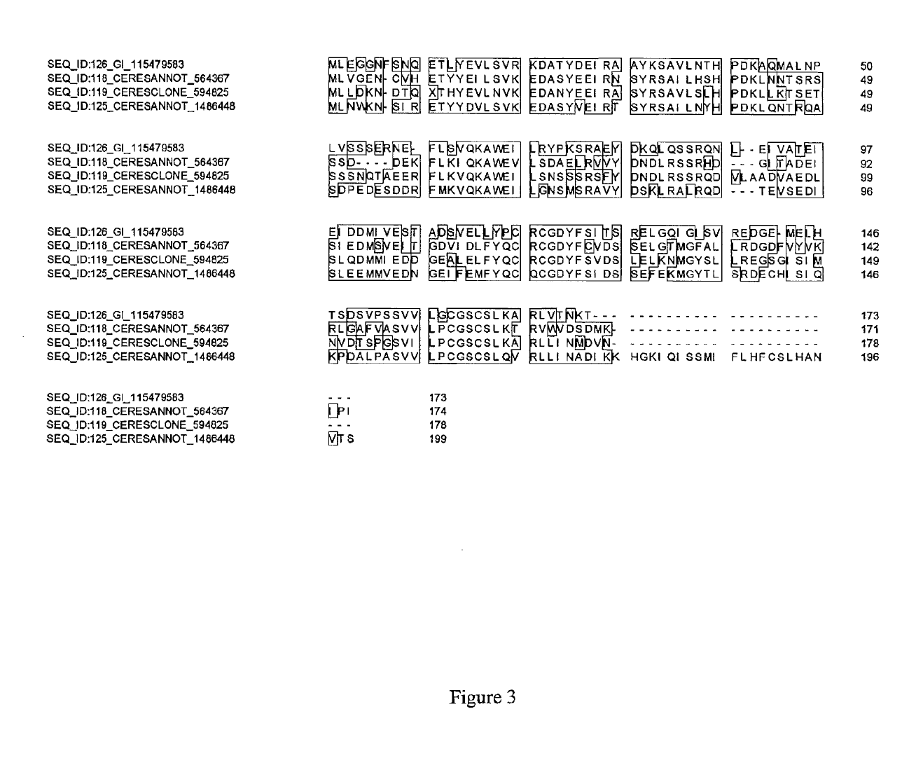

Amino acid sequences of homologs and/or orthologs of the polypeptide having the amino acid sequence set forth in SEQ ID NO:118 are provided in FIG. 3 and in the sequence listing. For example, the alignment in FIG. 3 provides the amino acid sequences of Lead Ceres ANNOT ID 564367 (SEQ ID NO:118), CeresClone:594825 (SEQ ID NO:119), CeresAnnot:1486448 (SEQ ID NO:125), and gi|115479583 (SEQ ID NO:126). Other homologs and/or orthologs of SEQ ID NO:118 include Ceres ANNOT ID 1539862 (SEQ ID NO:122), Ceres ANNOT ID 6068784 (SEQ ID NO:136), Ceres ANNOT ID 6041876 (SEQ ID NO:138), Ceres CLONE ID 333468 (SEQ ID NO:140), and gi/115476866 (SEQ ID NO:141).

In some cases, a protein-modulating polypeptide includes a polypeptide having at least 80% sequence identity, e.g., 80%, 85%, 90%, 95%, 97%, 98%, or 99% sequence identity, to an amino acid sequence corresponding to SEQ ID NO:119, SEQ ID NO:122, SEQ ID NO:125, SEQ ID NO:126, SEQ ID NO:136, SEQ ID NO:138, SEQ ID NO:140, and SEQ ID NO:141. SEQ ID NO:102 and SEQ ID NO:128 set forth the amino acid sequences of DNA clones, identified herein as Ceres ANNOT ID no. 571199 (SEQ ID NO:101) and Ceres ANNOT ID no. 851745 (SEQ ID NO:127), respectively, each of which is predicted to encode a polypeptide that does not have homology to an existing protein family based on Pfam analysis.

A protein-modulating polypeptide can comprise the amino acid sequence set forth in SEQ ID NO:102 and SEQ ID NO:128. Alternatively, a protein-modulating polypeptide can be a homolog, ortholog, or variant of the polypeptide having the amino acid sequence set forth in SEQ ID NO:102 or SEQ ID NO:128. For example, a protein-modulating polypeptide can have an amino acid sequence with at least 45% sequence identity, e.g., 47%, 50%, 55%, 60%, 65%, 70%, 75%, 80%, 85%, 90%, 95%, 97%, 98%, or 99% sequence identity, to the amino acid sequence set forth in SEQ ID NO:102 or SEQ ID NO:128

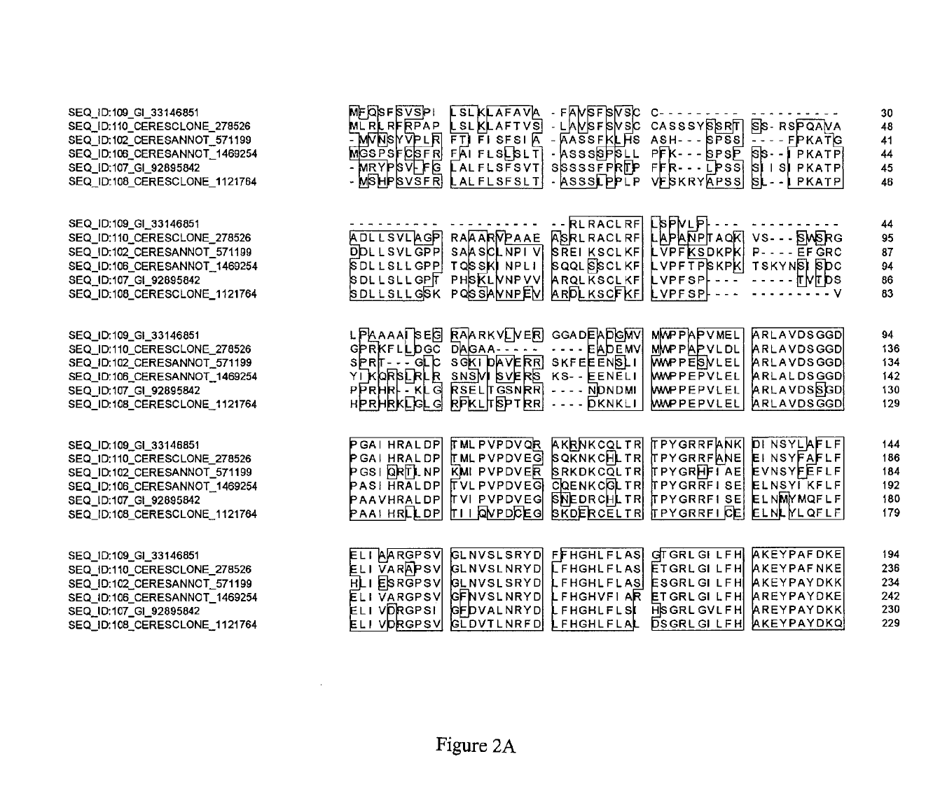

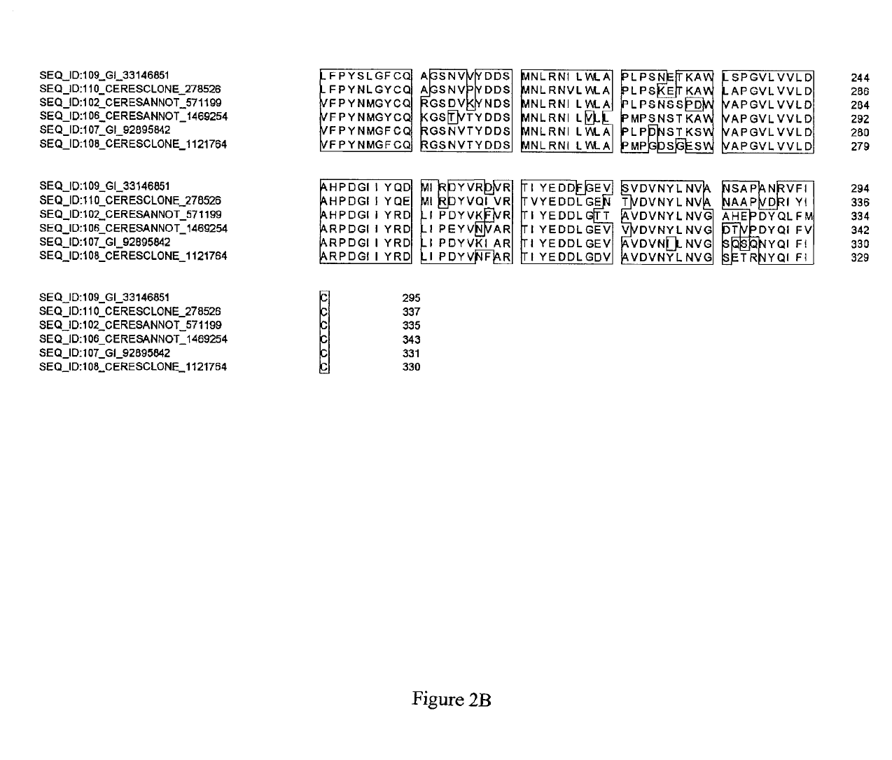

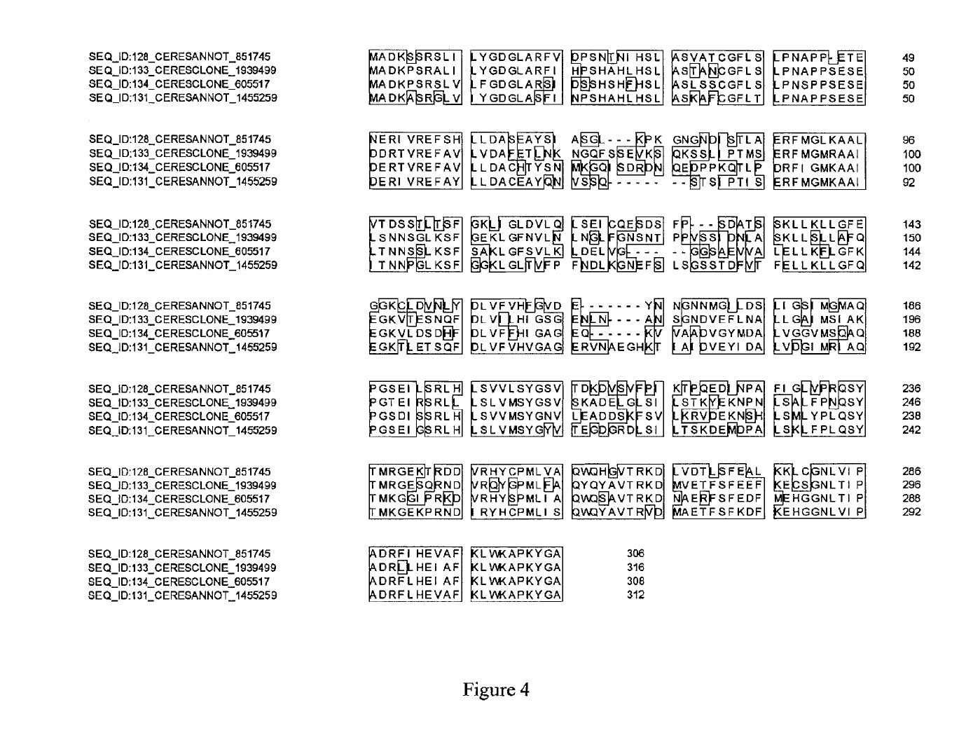

Amino acid sequences of homologs and/or orthologs of the polypeptide having the amino acid sequence set forth in SEQ ID NO:102 and SEQ ID NO:128 are provided in FIG. 2A-2B and FIG. 4, respectively, and in the sequence listing. For example, the alignment in FIGS. 2A-2B provides the amino acid sequences of Lead Annot ID 571199 (SEQ ID NO:102), CeresAnnot:1469254 (SEQ ID NO:106), gi|92895842 (SEQ ID NO:107), CeresClone:1121764 (SEQ ID NO:108), gi|33146851 (SEQ ID NO:109), and CeresClone:278526 (SEQ ID NO:110). Other homologs and/or orthologs include Public GI no. 110737799 (SEQ ID NO:103).

The alignment in FIG. 4 provides the amino acid sequences of Lead Ceres ANNOT ID 851745 (SEQ ID NO:128), CeresAnnot:1455259 (SEQ ID NO:131), CeresClone:1939499 (SEQ ID NO:133), and CeresClone:605517 (SEQ ID NO:134). Other homologs and/or orthologs of SEQ ID NO:128 include Ceres ANNOT ID 6007789 (SEQ ID NO:177), Ceres CLONE ID 842076 (SEQ ID NO:179), and gi|115444077 (SEQ ID NO:180).

In some cases, a protein-modulating polypeptide includes a polypeptide having at least 80% sequence identity, e.g., 80%, 85%, 90%, 95%, 97%, 98%, or 99% sequence identity, to an amino acid sequence corresponding to SEQ ID NO:103, SEQ ID NO:106, SEQ ID NO:107, SEQ ID NO:108, SEQ ID NO:109, SEQ ID NO:110, SEQ ID NO:131, SEQ ID NO:133, SEQ ID NO:134.

A protein-modulating polypeptide can contain an adh_short domain characteristic of polypeptides in the short-chain dehydrogenases/reductases family (SDR). This is a large family of enzymes, most of which are NAD- or NADP-dependent oxidoreductases. SEQ ID NO:181 sets forth the amino acid sequence of a Zea mays clone, identified herein as full-length Ceres Clone 258034 (SEQ ID NO:248), that is predicted to encode a polypeptide containing an adh_short domain. SEQ ID NO:116 sets forth the amino acid sequence of a chimeric polypeptide. Residues 1-45 of SEQ ID NO:116 correspond to residues 1-45 of SEQ ID NO:181. Residues 46-68 of SEQ ID NO:116 correspond to the predicted read-through translational product of vector sequence.

A protein-modulating polypeptide can comprise the amino acid sequence set forth in SEQ ID NO:116 or SEQ ID NO:181. Alternatively, a protein-modulating polypeptide can be a homolog, ortholog, or variant of the polypeptide having the amino acid sequence set forth in SEQ ID NO:181. For example, a protein-modulating polypeptide can have an amino acid sequence with at least 45% sequence identity, e.g., 46%, 50%, 55%, 60%, 65%, 70%, 75%, 80%, 85%, 90%, 95%, 97%, 98%, or 99% sequence identity, to the amino acid sequence set forth in SEQ ID NO:116 or SEQ ID NO:181.

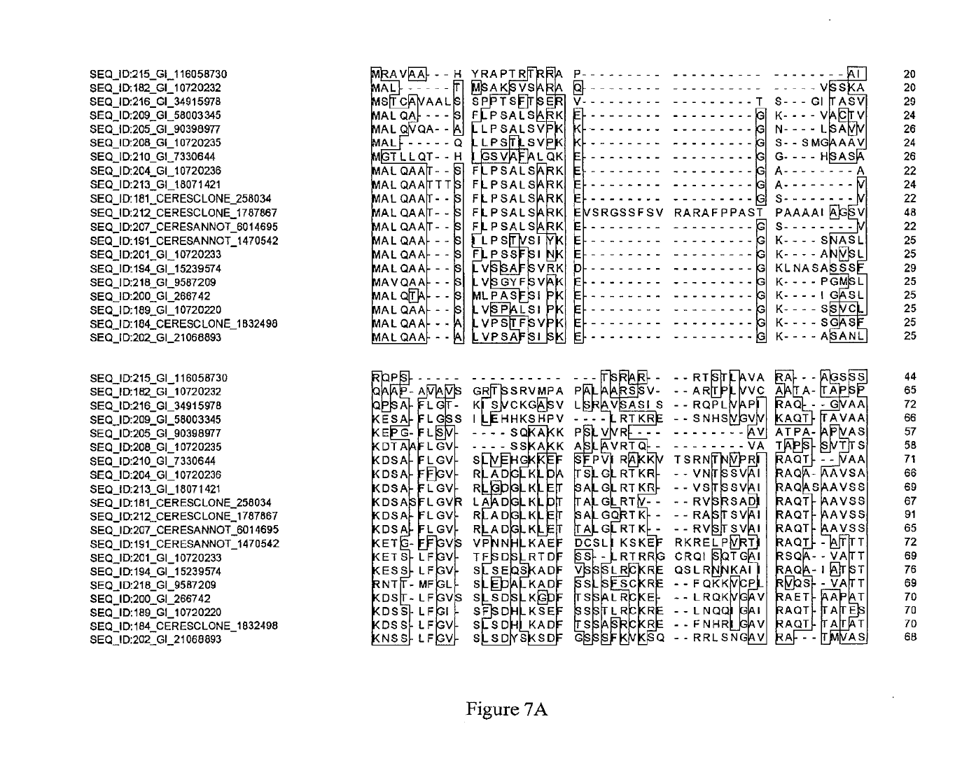

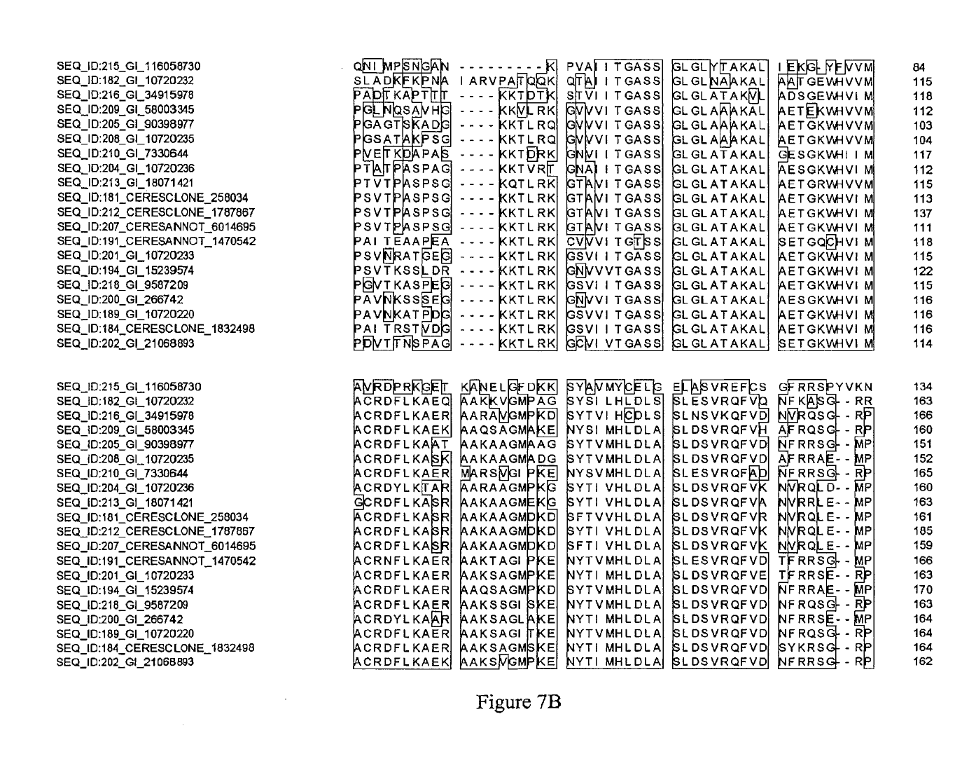

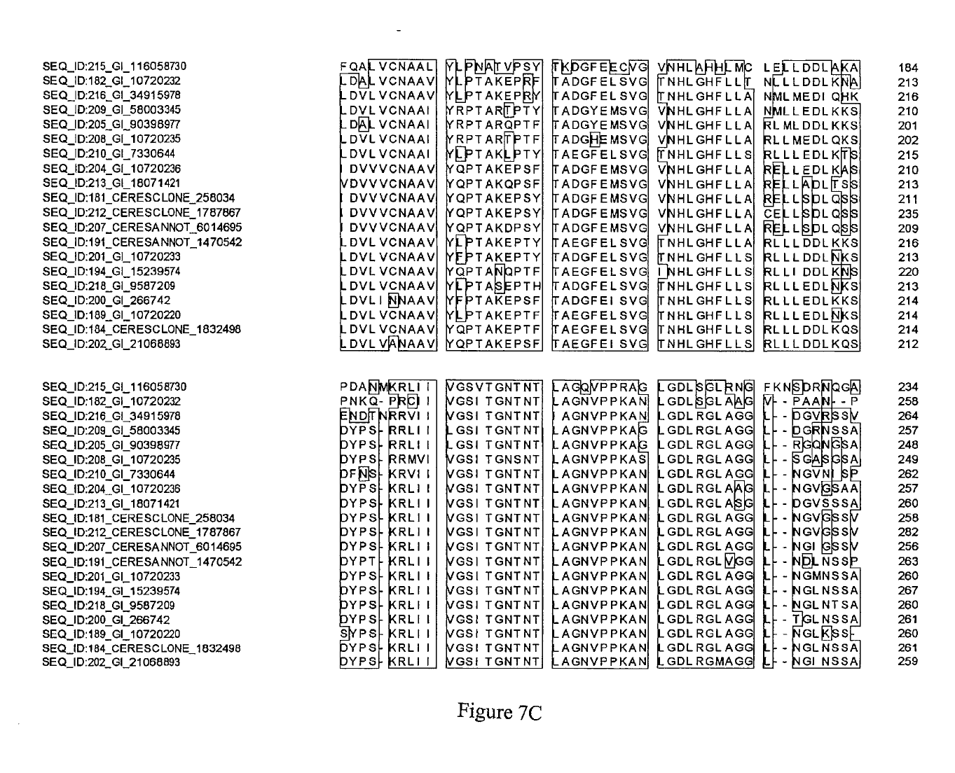

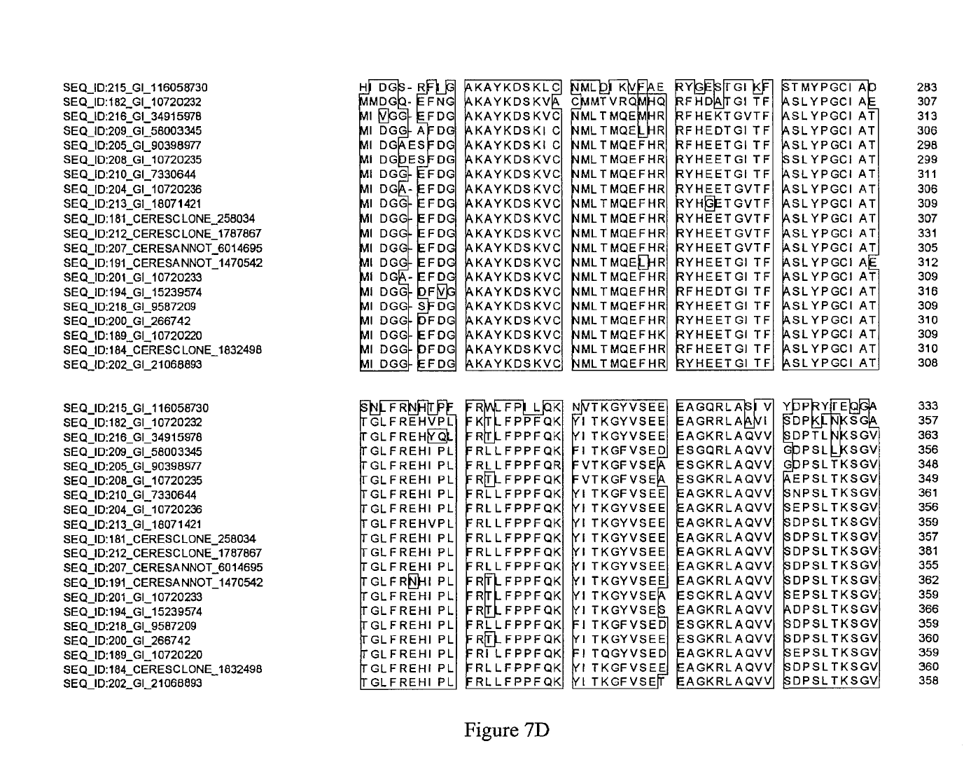

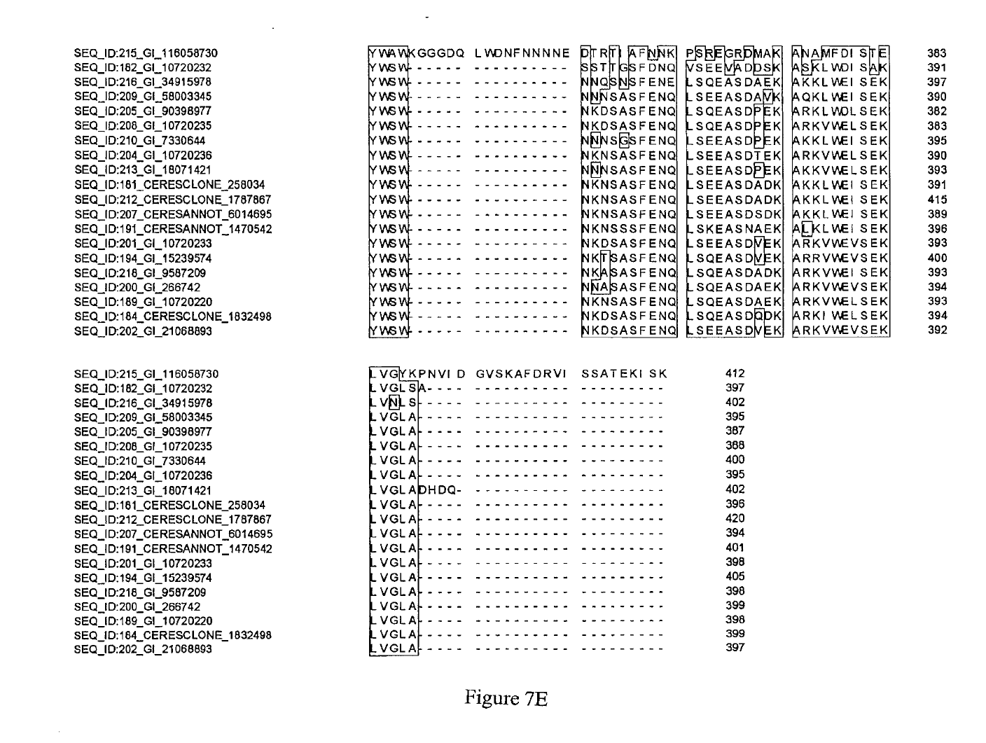

Amino acid sequences of homologs and/or orthologs of the polypeptide having the amino acid sequence set forth in SEQ ID NO:181 are provided in FIGS. 7A-7E and in the sequence listing. For example, the alignment in FIGS. 7A-7E provides the amino acid sequences of full-length Ceres CLONE ID 258034 (SEQ ID NO:181), gi|10720232 (SEQ ID NO:182), Ceres CLONE ID 1832498 (SEQ ID NO:184), gi|10720220 (SEQ ID NO:189), Ceres ANNOT ID 1470542 (SEQ ID NO:191), gi|15239574 (SEQ ID NO:194), gi|266742 (SEQ ID NO:200), gi|10720233 (SEQ ID NO:201), gi|21068893 (SEQ ID NO:202), gi|10720236 (SEQ ID NO:204), gi|90398977 (SEQ ID NO:205), Ceres ANNOT ID no. 6014695 (SEQ ID NO:207), gi|10720235 (SEQ ID NO:208), gi|58003345 (SEQ ID NO:209), gi|7330644 (SEQ ID NO:210), Ceres CLONE ID 1787867 (SEQ ID NO:212), gi|18071421 (SEQ ID NO:213), gi|116058730 (SEQ ID NO:215), gi|34915978 (SEQ ID NO:216), and gi|9587209 (SEQ ID NO:218). Other homologs and/or orthologs of SEQ ID NO:181 include Ceres CLONE ID no. 1846862 (SEQ ID NO:186), Ceres CLONE ID 1836128 (SEQ ID NO:188), Ceres ANNOT ID 1466766 (SEQ ID NO:193), gi|968975 (SEQ ID NO:195), gi|15234129 (SEQ ID NO:196), gi|79316418 (SEQ ID NO:197), gi|21593167 (SEQ ID NO:198), gi|15218860 (SEQ ID NO:199), gi|21068895 (SEQ ID NO:203), gi|10720231 (SEQ ID NO:214), gi|34915980 (SEQ ID NO:217), and gi|13699822 (SEQ ID NO:219).

In some cases, a protein-modulating polypeptide includes a polypeptide having at least 80% sequence identity, e.g., 80%, 85%, 90%, 95%, 97%, 98%, or 99% sequence identity, to an amino acid sequence corresponding to SEQ ID NO:182, SEQ ID NO:184, SEQ ID NO:186, SEQ ID NO:188, SEQ ID NO:189, SEQ ID NO:191, SEQ ID NO:193, SEQ ID NO:194, SEQ ID NO:195, SEQ ID NO:196, SEQ ID NO:197, SEQ ID NO:198, SEQ ID NO:199, SEQ ID NO:200, SEQ ID NO:201, SEQ ID NO:202, SEQ ID NO:203, SEQ ID NO:204, SEQ ID NO:205, SEQ ID NO:207, SEQ ID NO:208, SEQ ID NO:209, SEQ ID NO:210, SEQ ID NO:212, SEQ ID NO:213, SEQ ID NO:214, SEQ ID NO:215, SEQ ID NO:216, SEQ ID NO:217, or SEQ ID NO:218.

In some embodiments, a protein-modulating polypeptide is truncated at the amino- or carboxy-terminal end of a naturally occurring polypeptide. A truncated polypeptide may retain certain domains of the naturally occurring polypeptide while lacking others. Thus, length variants that are up to 5 amino acids shorter or longer typically exhibit the protein-modulating activity of a truncated polypeptide. In some embodiments, a truncated polypeptide is a dominant negative polypeptide. SEQ ID NO: 116 sets forth the amino sequence of a protein-modulating polypeptide that is truncated at the C-terminal end relative to the naturally occurring polypeptide (see SEQ ID NO:181). Expression in a plant of such a truncated polypeptide confers a difference in the level of protein in a tissue of the plant as compared to the corresponding level in tissue of a control plant that does not comprise the truncation

A protein-modulating polypeptide encoded by a recombinant nucleic acid can be a native protein-modulating polypeptide, i.e., one or more additional copies of the coding sequence for a protein-modulating polypeptide that is naturally present in the cell. Alternatively, a protein-modulating polypeptide can be heterologous to the cell, e.g., a transgenic Lycopersicon plant can contain the coding sequence for a kinase polypeptide from a Glycine plant.

A protein-modulating polypeptide can include additional amino acids that are not involved in protein modulation, and thus can be longer than would otherwise be the case. For example, a protein-modulating polypeptide can include an amino acid sequence that functions as a reporter. Such a protein-modulating polypeptide can be a fusion protein in which a green fluorescent protein (GFP) polypeptide is fused to, e.g., SEQ ID NO:112, or in which a yellow fluorescent protein (YFP) polypeptide is fused to, e.g., SEQ ID NO:118. In some embodiments, a protein-modulating polypeptide includes a purification tag, a chloroplast transit peptide, a mitochondrial transit peptide, or a leader sequence added to the amino or carboxy terminus.

Protein-modulating polypeptide candidates suitable for use in the invention can be identified by analysis of nucleotide and polypeptide sequence alignments. For example, performing a query on a database of nucleotide or polypeptide sequences can identify homologs and/or orthologs of protein-modulating polypeptides. Sequence analysis can involve BLAST, Reciprocal BLAST, or PSI-BLAST analysis of nonredundant databases using known protein-modulating polypeptide amino acid sequences. Those polypeptides in the database that have greater than 40% sequence identity can be identified as candidates for further evaluation for suitability as a protein-modulating polypeptide. Amino acid sequence similarity allows for conservative amino acid substitutions, such as substitution of one hydrophobic residue for another or substitution of one polar residue for another. If desired, manual inspection of such candidates can be carried out in order to narrow the number of candidates to be further evaluated. Manual inspection can be performed by selecting those candidates that appear to have domains suspected of being present in protein-modulating polypeptides, e.g., conserved functional domains.

The identification of conserved regions in a template or subject polypeptide can facilitate production of variants of wild type protein-modulating polypeptides. Conserved regions can be identified by locating a region within the primary amino acid sequence of a template polypeptide that is a repeated sequence, forms some secondary structure (e.g., helices and beta sheets), establishes positively or negatively charged domains, or represents a protein motif or domain. See, e.g., the Pfam web site describing consensus sequences for a variety of protein motifs and domains on the World Wide Web at sanger.ac.uk/Software/Pfam/ and pfam.janelia.org/. A description of the information included at the Pfam database is described in Sonnhammer et al., Nucl. Acids Res., 26:320-322 (1998); Sonnhammer et al., Proteins, 28:405-420 (1997); and Bateman et al., Nucl. Acids Res., 27:260-262 (1999). Amino acid residues corresponding to Pfam domains included in protein-modulating polypeptides provided herein are set forth in the sequence listing. For example, amino acid residues 158 to 296 of the amino acid sequence set forth in SEQ ID NO:96 correspond to a TLD domain, as indicated in fields <222> and <223> for SEQ ID NO:96 in the sequence listing.

Conserved regions also can be determined by aligning sequences of the same or related polypeptides from closely related species. Closely related species preferably are from the same family. In some embodiments, alignment of sequences from two different species is adequate. For example, sequences from Arabidopsis and Zea mays can be used to identify one or more conserved regions.

Typically, polypeptides that exhibit at least about 40% amino acid sequence identity are useful to identify conserved regions. Conserved regions of related polypeptides can exhibit at least 45% amino acid sequence identity (e.g., at least 50%, at least 60%, at least 70%, at least 80%, or at least 90% amino acid sequence identity). In some embodiments, a conserved region of target and template polypeptides exhibit at least 92%, 94%, 96%, 98%, or 99% amino acid sequence identity. Amino acid sequence identity can be deduced from amino acid or nucleotide sequences. In certain cases, highly conserved domains have been identified within protein-modulating polypeptides. These conserved regions can be useful in identifying functionally similar (orthologous) protein-modulating polypeptides.

In some instances, suitable protein-modulating polypeptides can be synthesized on the basis of consensus functional domains and/or conserved regions in polypeptides that are homologous protein-modulating polypeptides. Domains are groups of substantially contiguous amino acids in a polypeptide that can be used to characterize protein families and/or parts of proteins. Such domains have a “fingerprint” or “signature” that can comprise conserved (1) primary sequence, (2) secondary structure, and/or (3) three-dimensional conformation. Generally, domains are correlated with specific in vitro and/or in vivo activities. A domain can have a length of from 10 amino acids to 400 amino acids, e.g., 10 to 50 amino acids, or 25 to 100 amino acids, or 35 to 65 amino acids, or 35 to 55 amino acids, or 45 to 60 amino acids, or 200 to 300 amino acids, or 300 to 400 amino acids.

Representative homologs and/or orthologs of protein-modulating polypeptides are shown in FIGS. 1-7. Each Figure represents an alignment of the amino acid sequence of a protein-modulating polypeptide with the amino acid sequences of corresponding homologs and/or orthologs. Amino acid sequences of protein-modulating polypeptides and their corresponding homologs and/or orthologs have been aligned to identify conserved amino acids as shown in FIGS. 1-7. A dash in an aligned sequence represents a gap, i.e., a lack of an amino acid at that position. Identical amino acids or conserved amino acid substitutions among aligned sequences are identified by boxes.

The identification of conserved regions in a protein-modulating polypeptide facilitates production of variants of protein-modulating polypeptides. Variants of protein-modulating polypeptides typically have 10 or fewer conservative amino acid substitutions within the primary amino acid sequence, e.g., 7 or fewer conservative amino acid substitutions, 5 or fewer conservative amino acid substitutions, or between 1 and 5 conservative substitutions. Useful polypeptides can be constructed based on the conserved regions in FIG. 1, FIG. 2, FIG. 3, FIG. 4, FIG. 5, FIG. 6, or FIG. 7. Such a polypeptide includes the conserved regions arranged in the order depicted in the Figure from amino-terminal end to carboxy-terminal end. Such a polypeptide may also include zero, one, or more than one amino acid in positions marked by dashes. When no amino acids are present at positions marked by dashes, the length of such a polypeptide is the sum of the amino acid residues in all conserved regions. When amino acids are present at all positions marked by dashes, such a polypeptide has a length that is the sum of the amino acid residues in all conserved regions and all dashes.

Consensus domains and conserved regions can be identified by homologous polypeptide sequence analysis as described above. The suitability of polypeptides for use as protein-modulating polypeptides can be evaluated by functional complementation studies.

A protein-modulating polypeptide also can be a fragment of a naturally occurring protein-modulating polypeptide. In certain cases, such as transcription factor protein-modulating polypeptides, a fragment can comprise the DNA-binding and transcription-regulating domains of the naturally occurring protein-modulating polypeptide. In some cases, such as enzyme protein-modulating polypeptides, a fragment can comprise the catalytic domain of the naturally occurring protein-modulating polypeptide.

Useful protein-modulating polypeptides also can include those that fit a Hidden Markov Model based on the polypeptides set forth in any one of FIGS. 1-7. A Hidden Markov Model (HMM) is a statistical model of a consensus sequence for a group of functional homologs. See, Durbin et al., Biological Sequence Analysis: Probabilistic Models of Proteins and Nucleic Acids, Cambridge University Press, Cambridge, UK (1998). An HMM is generated by the program HMMER 2.3.2 with default program parameters, using the sequences of the group of functional homologs as input. The multiple sequence alignment is generated by ProbCons (Do et al., Genome Res., 15(2):330-40 (2005)) version 1.11 using a set of default parameters: -c, —consistency REPS of 2; -ir, —iterative-refinement REPS of 100; -pre, —pre-training REPS of 0. ProbCons is a public domain software program provided by Stanford University.

The default parameters for building an HMM (hmmbuild) are as follows: the default “architecture prior” (archpri) used by MAP architecture construction is 0.85, and the default cutoff threshold (idlevel) used to determine the effective sequence number is 0.62. HMMER 2.3.2 was released Oct. 3, 2003 under a GNU general public license, and is available from various sources on the World Wide Web such as hmmer.janelia.org; hmmer.wustl.edu; and fr.com/hmmer232/. Hmmbuild outputs the model as a text file.

The HMM for a group of functional homologs can be used to determine the likelihood that a candidate protein-modulating polypeptide sequence is a better fit to that particular HMM than to a null HMM generated using a group of sequences that are not structurally or functionally related. The likelihood that a candidate polypeptide sequence is a better fit to an HMM than to a null HMM is indicated by the HMM bit score, a number generated when the candidate sequence is fitted to the HMM profile using the HMMER hmmsearch program. The following default parameters are used when running hmmsearch: the default E-value cutoff (E) is 10.0, the default bit score cutoff (T) is negative infinity, the default number of sequences in a database (Z) is the real number of sequences in the database, the default E-value cutoff for the per-domain ranked hit list (domE) is infinity, and the default bit score cutoff for the per-domain ranked hit list (domT) is negative infinity. A high HMM bit score indicates a greater likelihood that the candidate sequence carries out one or more of the biochemical or physiological function(s) of the polypeptides used to generate the HMM. A high HMM bit score is at least 20, and often is higher. Slight variations in the HMM bit score of a particular sequence can occur due to factors such as the order in which sequences are processed for alignment by multiple sequence alignment algorithms such as the ProbCons program. Nevertheless, such HMM bit score variation is minor.

The protein-modulating polypeptides discussed herein fit the indicated HMM with an HMM bit score greater than 20 (e.g., greater than 30, 40, 50, 60, 70, 80, 90, 100, 200, 300, 400, or 500). In some embodiments, the HMM bit score of a protein-modulating polypeptide discussed below is about 50%, 60%, 70%, 80%, 90%, or 95% of the HMM bit score of a functional homolog provided in one of FIGS. 1-7. In some embodiments, a protein-modulating polypeptide discussed herein fits the indicated HMM with an HMM bit score greater than 20, and has a conserved domain e.g., a PFAM domain indicative of a protein-modulating polypeptide discussed herein. In some embodiments, a protein-modulating polypeptide discussed herein fits the indicated HMM with an HMM bit score greater than 20, and has 70% or greater sequence identity (e.g., 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity) to an amino acid sequence shown in any one of FIGS. 1-7.

For example, a protein-modulating polypeptide can fit an HMM generated using the amino acid sequences set forth in FIG. 1 with an HMM bit score that is greater than about 820 (e.g., greater than about 844, 875, 900, 925, or 930). In some cases, a protein-modulating polypeptide can fit an HMM generated using the amino acid sequences set forth in FIG. 2 with an HMM bit score that is greater than about 680 (e.g., greater than about 690, 700, 720, 730, 740, 750, 775, 800, 825, 850, 870, 875, 890, or 895). In some cases, a protein-modulating polypeptide can fit an HMM generated using the amino acid sequences set forth in FIG. 3 with an HMM bit score that is greater than about 225 (e.g., greater than about 230, 235, 250, 275, 300, 325, 330, 340, 345, 375, 400, 425, 450, 475, 500, 525, 550, 560, 575, or 585). In some cases, a protein-modulating polypeptide can fit an HMM generated using the amino acid sequences set forth in FIG. 4 with an HMM bit score that is greater than about 800 (e.g., greater than about 805, 810, 815, 820, 825, 830, 840, 850, or 860). In some cases, a protein-modulating polypeptide can fit an HMM generated using the amino acid sequences set forth in FIG. 5 with an HMM bit score that is greater than about 500 (e.g., greater than about 510, 525, 550, 575, 600, 625, 650, 675, or 700). In some cases, a protein-modulating polypeptide can fit an HMM generated using the amino acid sequences set forth in FIG. 6 with an HMM bit score that is greater than about 1140 (e.g., greater than about 1150, 1200, 1250, 1300, 1350, 1375, 1400, 1450, 1475, 1500, 1550, 1575, 1600, 1650, 1675, 1700, 1750, 1775, 1800, 1850, 1875, 1900, 1950, 1975, 2000, 2010, or 2015). In some cases, a protein-modulating polypeptide can fit an HMM generated using the amino acid sequences set forth in FIG. 7 with an HMM bit score that is greater than about 840 (e.g., greater than about 845, 850, 875, 900, 925, 950, 975, 1000, 1025, 1050, 1075, 1100, 1125, 1130, or 1135).

Nucleic Acids

The terms “nucleic acid” and “polynucleotide” are used interchangeably herein, and refer to both RNA and DNA, including cDNA, genomic DNA, synthetic DNA, and DNA (or RNA) containing nucleic acid analogs. Polynucleotides can have any three-dimensional structure. A nucleic acid can be double-stranded or single-stranded (i.e., a sense strand or an antisense strand). Non-limiting examples of polynucleotides include genes, gene fragments, exons, introns, messenger RNA (mRNA), transfer RNA, ribosomal RNA, siRNA, micro-RNA, ribozymes, cDNA, recombinant polynucleotides, branched polynucleotides, plasmids, vectors, isolated DNA of any sequence, isolated RNA of any sequence, nucleic acid probes, and primers, as well as nucleic acid analogs.

Nucleic acids described herein include protein-modulating nucleic acids. Protein-modulating nucleic acids can be effective to modulate protein levels when transcribed in a plant or plant cell. A protein-modulating nucleic acid can comprise the nucleotide sequence set forth in SEQ ID NO:115. Alternatively, a protein-modulating nucleic acid can be a variant of the nucleic acid having the nucleotide sequence set forth in SEQ ID NO:115. For example, a protein-modulating nucleic acid can have a nucleotide sequence with at least 80% sequence identity, e.g., 81%, 85%, 90%, 95%, 97%, 98%, or 99% sequence identity, to the nucleotide sequence set forth in SEQ ID NO:115.

An “isolated nucleic acid” can be, for example, a naturally-occurring DNA molecule, provided one of the nucleic acid sequences normally found immediately flanking that DNA molecule in a naturally-occurring genome is removed or absent. Thus, an isolated nucleic acid includes, without limitation, a DNA molecule that exists as a separate molecule, independent of other sequences (e.g., a chemically synthesized nucleic acid, or a cDNA or genomic DNA fragment produced by the polymerase chain reaction (PCR) or restriction endonuclease treatment). An isolated nucleic acid also refers to a DNA molecule that is incorporated into a vector, an autonomously replicating plasmid, a virus, or into the genomic DNA of a prokaryote or eukaryote. In addition, an isolated nucleic acid can include an engineered nucleic acid such as a DNA molecule that is part of a hybrid or fusion nucleic acid. A nucleic acid existing among hundreds to millions of other nucleic acids within, for example, cDNA libraries or genomic libraries, or gel slices containing a genomic DNA restriction digest, is not to be considered an isolated nucleic acid.

Isolated nucleic acid molecules can be produced by standard techniques. For example, polymerase chain reaction (PCR) techniques can be used to obtain an isolated nucleic acid containing a nucleotide sequence described herein. PCR can be used to amplify specific sequences from DNA as well as RNA, including sequences from total genomic DNA or total cellular RNA. Various PCR methods are described, for example, in PCR Primer: A Laboratory Manual, Dieffenbach and Dveksler, eds., Cold Spring Harbor Laboratory Press, 1995. Generally, sequence information from the ends of the region of interest or beyond is employed to design oligonucleotide primers that are identical or similar in sequence to opposite strands of the template to be amplified. Various PCR strategies also are available by which site-specific nucleotide sequence modifications can be introduced into a template nucleic acid. Isolated nucleic acids also can be chemically synthesized, either as a single nucleic acid molecule (e.g., using automated DNA synthesis in the 3′ to 5′ direction using phosphoramidite technology) or as a series of oligonucleotides. For example, one or more pairs of long oligonucleotides (e.g., >100 nucleotides) can be synthesized that contain the desired sequence, with each pair containing a short segment of complementarity (e.g., about 15 nucleotides) such that a duplex is formed when the oligonucleotide pair is annealed. DNA polymerase is used to extend the oligonucleotides, resulting in a single, double-stranded nucleic acid molecule per oligonucleotide pair, which then can be ligated into a vector. Isolated nucleic acids of the invention also can be obtained by mutagenesis of, e.g., a naturally occurring DNA.

The term “percent sequence identity” refers to the degree of identity between any given query sequence, e.g., SEQ ID NO:102, and a subject sequence. A subject sequence typically has a length that is from 80 percent to 200 percent of the length of the query sequence, e.g., 82, 85, 87, 89, 90, 93, 95, 97, 99, 100, 105, 110, 115, 120, 130, 140, 150, 160, 170, 180, 190, or 200 percent of the length of the query sequence. A percent identity for any subject nucleic acid or polypeptide relative to a query nucleic acid or polypeptide can be determined as follows. A query sequence (e.g., a nucleic acid sequence or an amino acid sequence) is aligned to one or more subject sequences using the computer program ClustalW (version 1.83, default parameters), which allows alignments of nucleic acid or polypeptide sequences to be carried out across their entire length (global alignment). Chema et al., Nucleic Acids Res., 31(13):3497-500 (2003).

ClustalW calculates the best match between a query and one or more subject sequences, and aligns them so that identities, similarities and differences can be determined. Gaps of one or more residues can be inserted into a query sequence, a subject sequence, or both, to maximize sequence alignments. For fast pairwise alignment of nucleic acid sequences, the following default parameters are used: word size: 2; window size: 4; scoring method: percentage; number of top diagonals: 4; and gap penalty: 5. For multiple alignment of nucleic acid sequences, the following parameters are used: gap opening penalty: 10.0; gap extension penalty: 5.0; and weight transitions: yes. For fast pairwise alignment of protein sequences, the following parameters are used: word size: 1; window size: 5; scoring method: percentage; number of top diagonals: 5; gap penalty: 3. For multiple alignment of protein sequences, the following parameters are used: weight matrix: blosum; gap opening penalty: 10.0; gap extension penalty: 0.05; hydrophilic gaps: on; hydrophilic residues: Gly, Pro, Ser, Asn, Asp, Gln, Glu, Arg, and Lys; residue-specific gap penalties: on. The ClustalW output is a sequence alignment that reflects the relationship between sequences. ClustalW can be run, for example, at the Baylor College of Medicine Search Launcher site (searchlauncher.bcm.tmc.edu/multi-align/multi-align.html) and at the European Bioinformatics Institute site on the World Wide Web (ebi.ac.uk/clustalw).

To determine percent identity of a subject nucleic acid or amino acid sequence to a query sequence, the sequences are aligned using ClustalW, the number of identical matches in the alignment is divided by the length of the query sequence, and the result is multiplied by 100. It is noted that the percent identity value can be rounded to the nearest tenth. For example, 78.11, 78.12, 78.13, and 78.14 are rounded down to 78.1, while 78.15, 78.16, 78.17, 78.18, and 78.19 are rounded up to 78.2.

The term “exogenous” with respect to a nucleic acid indicates that the nucleic acid is part of a recombinant nucleic acid construct, or is not in its natural environment. For example, an exogenous nucleic acid can be a sequence from one species introduced into another species, i.e., a heterologous nucleic acid. Typically, such an exogenous nucleic acid is introduced into the other species via a recombinant nucleic acid construct. An exogenous nucleic acid can also be a sequence that is native to an organism and that has been reintroduced into cells of that organism. An exogenous nucleic acid that includes a native sequence can often be distinguished from the naturally occurring sequence by the presence of non-natural sequences linked to the exogenous nucleic acid, e.g., non-native regulatory sequences flanking a native sequence in a recombinant nucleic acid construct. In addition, stably transformed exogenous nucleic acids typically are integrated at positions other than the position where the native sequence is found. It will be appreciated that an exogenous nucleic acid may have been introduced into a progenitor and not into the cell under consideration. For example, a transgenic plant containing an exogenous nucleic acid can be the progeny of a cross between a stably transformed plant and a non-transgenic plant. Such progeny are considered to contain the exogenous nucleic acid.

Recombinant constructs are also provided herein and can be used to transform plants or plant cells in order to modulate protein levels. A recombinant nucleic acid construct can comprise a nucleic acid encoding a protein-modulating polypeptide as described herein, operably linked to a regulatory region suitable for expressing the protein-modulating polypeptide in the plant or cell. Thus, a nucleic acid can comprise a coding sequence that encodes any of the protein-modulating polypeptides as set forth in SEQ ID NOs:96-98, SEQ ID NO:100, SEQ ID NOs:102-103, SEQ ID NOs:106-110, SEQ ID NO:112, SEQ ID NO:114, SEQ ID NO:116, SEQ ID NOs:118-119, SEQ ID NO:122, SEQ ID NOs:125-126, SEQ ID NO:128, SEQ ID NO:131, SEQ ID NOs:133-134, SEQ ID NO:136, SEQ ID NO:138, SEQ ID NOs:140-141, SEQ ID NOs:143-146, SEQ ID NO:148, SEQ ID NO:150, SEQ ID NO:152, SEQ ID NO:154, SEQ ID NOs:156-157, SEQ ID NO:159, SEQ ID NO:161, SEQ ID NO:163, SEQ ID NO:165, SEQ ID NOs:167-175, SEQ ID NO:177, SEQ ID NOs:179-182, SEQ ID NO:184, SEQ ID NO:186, SEQ ID NO:186, SEQ ID NOs:188-189, SEQ ID NO:191, SEQ ID NOs:193-205, SEQ ID NOs:207-210, SEQ ID NOs:212-220, SEQ ID NO:222, SEQ ID NO:224, SEQ ID NO:226, SEQ ID NO:228, SEQ ID NO:230, SEQ ID NO:232, SEQ ID NO:234, SEQ ID NO:236, SEQ ID NOs:238-239, SEQ ID NO:241, and SEQ ID NOs:243-247.

Examples of nucleic acids encoding protein-modulating polypeptides are set forth in SEQ ID NO:95, SEQ ID NO:99, SEQ ID NO:101, SEQ ID NO:104, SEQ ID NO:105, SEQ ID NO:111, SEQ ID NO:113, SEQ ID NO:115, SEQ ID NO:117, SEQ ID NO:120, SEQ ID NO:121, SEQ ID NO:123, SEQ ID NO:124, SEQ ID NO:127, SEQ ID NO:129, SEQ ID NO:130, SEQ ID NO:132, SEQ ID NO:135, SEQ ID NO:137, SEQ ID NO:139, SEQ ID NO:142, SEQ ID NO:147, SEQ ID NO:149, SEQ ID NO:151, SEQ ID NO:153, SEQ ID NO:155, SEQ ID NO:158, SEQ ID NO:160, SEQ ID NO:162, SEQ ID NO:164, SEQ ID NO:166, SEQ ID NO:176, SEQ ID NO:178, SEQ ID NO:183, SEQ ID NO:187, SEQ ID NO:190, SEQ ID NO:192, SEQ ID NO:206, SEQ ID NO:211, SEQ ID NO:221, SEQ ID NO:225, SEQ ID NO:227, SEQ ID NO:292, SEQ ID NO:231, SEQ ID NO:233, SEQ ID NO:235, SEQ ID NO:237, SEQ ID NO:237, SEQ ID NO:240, SEQ ID NO:242, and SEQ ID NO:248.

A nucleic acid also can be a fragment that is at least 40% (e.g., at least 45, 50, 55, 60, 65, 70, 75, 80, 85, 90, 95, or 99%) of the length of the nucleic acid set forth in SEQ ID NO:95, SEQ ID NO:99, SEQ ID NO:101, SEQ ID NO:104, SEQ ID NO:105, SEQ ID NO:111, SEQ ID NO:113, SEQ ID NO:115, SEQ ID NO:117, SEQ ID NO:120, SEQ ID NO:121, SEQ ID NO:123, SEQ ID NO:124, SEQ ID NO:127, SEQ ID NO:129, SEQ ID NO:130, SEQ ID NO:132, SEQ ID NO:135, SEQ ID NO:137, SEQ ID NO:139, SEQ ID NO:142, SEQ ID NO:147, SEQ ID NO:149, SEQ ID NO:151, SEQ ID NO:153, SEQ ID NO:155, SEQ ID NO:158, SEQ ID NO:160, SEQ ID NO:162, SEQ ID NO:164, SEQ ID NO:166, SEQ ID NO:176, SEQ ID NO:178, SEQ ID NO:183, SEQ ID NO:187, SEQ ID NO:190, SEQ ID NO:192, SEQ ID NO:206, SEQ ID NO:211, SEQ ID NO:221, SEQ ID NO:225, SEQ ID NO:227, SEQ ID NO:292, SEQ ID NO:231, SEQ ID NO:233, SEQ ID NO:235, SEQ ID NO:237, SEQ ID NO:237, SEQ ID NO:240, SEQ ID NO:242, and SEQ ID NO:248.

SEQ ID NO:95 is predicted to encode a polypeptide having the amino acid sequence set forth in SEQ ID NO:96. SEQ ID NO:99 is predicted to encode a polypeptide having the amino acid sequence set forth in SEQ ID NO:100. SEQ ID NO:101 is predicted to encode a polypeptide having the amino acid sequence set forth in SEQ ID NO:102 SEQ ID NO:104 is predicted to encode the polypeptide having the amino acid sequence set forth in SEQ ID NO:105. SEQ ID NO:111 is predicted to encode the polypeptide having the amino acid sequence set forth in SEQ ID NO:112. SEQ ID NO:113 is predicted to encode the polypeptide having the amino acid sequence set forth in SEQ ID NO:114. SEQ ID NO:115 is predicted to encode the polypeptide having the amino acid sequence set forth in SEQ ID NO:116. SEQ ID NO:117 is predicted to encode the polypeptide having the amino acid sequence set forth in SEQ ID NO:118. SEQ ID NO:121 is predicted to encode the polypeptide having the amino acid sequence set forth in SEQ ID NO:122. SEQ ID NO:124 is predicted to encode the polypeptide having the amino acid sequence set forth in SEQ ID NO:125. SEQ ID NO:127 is predicted to encode the polypeptide having the amino acid sequence set forth in SEQ ID NO:128. SEQ ID NO:130 is predicted to encode the polypeptide having the amino acid sequence set forth in SEQ ID NO:131. SEQ ID NO:132 is predicted to encode the polypeptide having the amino acid sequence set forth in SEQ ID NO:133. SEQ ID NO:135 is predicted to encode the polypeptide having the amino acid sequence set forth in SEQ ID NO:136. SEQ ID NO:137 is predicted to encode the polypeptide having the amino acid sequence set forth in SEQ ID NO:138. SEQ ID NO:139 is predicted to encode the polypeptide having the amino acid sequence set forth in SEQ ID NO:140. SEQ ID NO:142 is predicted to encode the polypeptide having the amino acid sequence set forth in SEQ ID NO:143. SEQ ID NO:147 is predicted to encode the polypeptide having the amino acid sequence set forth in SEQ ID NO:148. SEQ ID NO:149 is predicted to encode the polypeptide having the amino acid sequence set forth in SEQ ID NO:150. SEQ ID NO:151 is predicted to encode the polypeptide having the amino acid sequence set forth in SEQ ID NO:152. SEQ ID NO:153 is predicted to encode the polypeptide having the amino acid sequence set forth in SEQ ID NO:154. SEQ ID NO:155 is predicted to encode the polypeptide having the amino acid sequence set forth in SEQ ID NO:156. SEQ ID NO:158 is predicted to encode the polypeptide having the amino acid sequence set forth in SEQ ID NO:159. SEQ ID NO:160 is predicted to encode the polypeptide having the amino acid sequence set forth in SEQ ID NO:161. SEQ ID NO:162 is predicted to encode the polypeptide having the amino acid sequence set forth in SEQ ID NO:163. SEQ ID NO:164 is predicted to encode the polypeptide having the amino acid sequence set forth in SEQ ID NO:165. SEQ ID NO:166 is predicted to encode the polypeptide having the amino acid sequence set forth in SEQ ID NO:167. SEQ ID NO:176 is predicted to encode the polypeptide having the amino acid sequence set forth in SEQ ID NO:177. SEQ ID NO:178 is predicted to encode the polypeptide having the amino acid sequence set forth in SEQ ID NO:179. SEQ ID NO:248 is predicted to encode the polypeptide having the amino acid sequence set forth in SEQ ID NO:181. SEQ ID NO:183 is predicted to encode the polypeptide having the amino acid sequence set forth in SEQ ID NO:184. SEQ ID NO:185 is predicted to encode the polypeptide having the amino acid sequence set forth in SEQ ID NO:186. SEQ ID NO:187 is predicted to encode the polypeptide having the amino acid sequence set forth in SEQ ID NO:188. SEQ ID NO:190 is predicted to encode the polypeptide having the amino acid sequence set forth in SEQ ID NO:191. SEQ ID NO:192 is predicted to encode the polypeptide having the amino acid sequence set forth in SEQ ID NO:193. SEQ ID NO:206 is predicted to encode the polypeptide having the amino acid sequence set forth in SEQ ID NO:207. SEQ ID NO:211 is predicted to encode the polypeptide having the amino acid sequence set forth in SEQ ID NO:212. SEQ ID NO:221 is predicted to encode the polypeptide having the amino acid sequence set forth in SEQ ID NO:222. SEQ ID NO:223 is predicted to encode the polypeptide having the amino acid sequence set forth in SEQ ID NO:224. SEQ ID NO:225 is predicted to encode the polypeptide having the amino acid sequence set forth in SEQ ID NO:226. SEQ ID NO:227 is predicted to encode the polypeptide having the amino acid sequence set forth in SEQ ID NO:228. SEQ ID NO:229 is predicted to encode the polypeptide having the amino acid sequence set forth in SEQ ID NO:230. SEQ ID NO:231 is predicted to encode the polypeptide having the amino acid sequence set forth in SEQ ID NO:232. SEQ ID NO:233 is predicted to encode the polypeptide having the amino acid sequence set forth in SEQ ID NO:234. SEQ ID NO:236 is predicted to encode the polypeptide having the amino acid sequence set forth in SEQ ID NO:237. SEQ ID NO:240 is predicted to encode the polypeptide having the amino acid sequence set forth in SEQ ID NO:241. SEQ ID NO:242 is predicted to encode the polypeptide having the amino acid sequence set forth in SEQ ID NO:243.

In some cases, a recombinant nucleic acid construct can include a nucleic acid comprising less than the full-length of a coding sequence. Typically, such a construct also includes a regulatory region operably linked to the protein-modulating nucleic acid.

It will be appreciated that a number of nucleic acids can encode a polypeptide having a particular amino acid sequence. The degeneracy of the genetic code is well known to the art; i.e., for many amino acids, there is more than one nucleotide triplet that serves as the codon for the amino acid. For example, codons in the coding sequence for a given protein-modulating polypeptide can be modified such that optimal expression in a particular plant species is obtained, using appropriate codon bias tables for that species.

Vectors containing nucleic acids such as those described herein also are provided. A “vector” is a replicon, such as a plasmid, phage, or cosmid, into which another DNA segment may be inserted so as to bring about the replication of the inserted segment. Generally, a vector is capable of replication when associated with the proper control elements. Suitable vector backbones include, for example, those routinely used in the art such as plasmids, viruses, artificial chromosomes, BACs, YACs, or PACs. The term “vector” includes cloning and expression vectors, as well as viral vectors and integrating vectors. An “expression vector” is a vector that includes a regulatory region. Suitable expression vectors include, without limitation, plasmids and viral vectors derived from, for example, bacteriophage, baculoviruses, and retroviruses. Numerous vectors and expression systems are commercially available from such corporations as Novagen (Madison, Wis.), Clontech (Palo Alto, Calif.), Stratagene (La Jolla, Calif.), and Invitrogen/Life Technologies (Carlsbad, Calif.).

The vectors provided herein also can include, for example, origins of replication, scaffold attachment regions (SARs), and/or markers. A marker gene can confer a selectable phenotype on a plant cell. For example, a marker can confer biocide resistance, such as resistance to an antibiotic (e.g., kanamycin, G418, bleomycin, or hygromycin), or an herbicide (e.g., chlorosulfuron or phosphinothricin). In addition, an expression vector can include a tag sequence designed to facilitate manipulation or detection (e.g., purification or localization) of the expressed polypeptide. Tag sequences, such as green fluorescent protein (GFP), glutathione S-transferase (GST), polyhistidine, c-myc, hemagglutinin, or Flag™ tag (Kodak, New Haven, Conn.) sequences typically are expressed as a fusion with the encoded polypeptide. Such tags can be inserted anywhere within the polypeptide, including at either the carboxyl or amino terminus.

Regulatory Regions

The term “regulatory region” refers to a nucleic acid having nucleotide sequences that influence transcription or translation initiation and rate, and stability and/or mobility of a transcription or translation product. Regulatory regions include, without limitation, promoter sequences, enhancer sequences, response elements, protein recognition sites, inducible elements, protein binding sequences, 5′ and 3′ untranslated regions (UTRs), transcriptional start sites, termination sequences, polyadenylation sequences, introns, and combinations thereof.

As used herein, the term “operably linked” refers to positioning of a regulatory region and a sequence to be transcribed in a nucleic acid so as to influence transcription or translation of such a sequence. For example, to bring a coding sequence under the control of a regulatory region, the translation initiation site of the translational reading frame of the polypeptide is typically positioned between one and about fifty nucleotides downstream of the regulatory region. A regulatory region can, however, be positioned as much as about 5,000 nucleotides upstream of the translation initiation site, or about 2,000 nucleotides upstream of the transcription start site. A regulatory region typically comprises at least a core (basal) promoter. A regulatory region also may include at least one control element, such as an enhancer sequence, an upstream element or an upstream activation region (UAR). For example, a suitable enhancer is a cis-regulatory element (−212 to −154) from the upstream region of the octopine synthase (ocs) gene. Fromm et al., The Plant Cell, 1:977-984 (1989). The choice of regulatory regions to be included depends upon several factors, including, but not limited to, efficiency, selectability, inducibility, desired expression level, and cell- or tissue-preferential expression. It is a routine matter for one of skill in the art to modulate the expression of a coding sequence by appropriately selecting and positioning regulatory regions relative to the coding sequence.

Some suitable regulatory regions initiate transcription only, or predominantly, in certain cell types. For example, a promoter that is active predominantly in a reproductive tissue (e.g., fruit, ovule, pollen, pistils, female gametophyte, egg cell, central cell, nucellus, suspensor, synergid cell, flowers, embryonic tissue, embryo sac, embryo, zygote, endosperm, integument, or seed coat) can be used. Thus, as used herein a cell type- or tissue-preferential promoter is one that drives expression preferentially in the target tissue, but may also lead to some expression in other cell types or tissues as well. Methods for identifying and characterizing regulatory regions in plant genomic DNA include, for example, those described in the following references: Jordano et al., Plant Cell, 1:855-866 (1989); Bustos et al., Plant Cell, 1:839-854 (1989); Green et al., EMBO J., 7:4035-4044 (1988); Meier et al., Plant Cell, 3:309-316 (1991); and Zhang et al., Plant Physiology, 110:1069-1079 (1996).

Examples of various classes of regulatory regions are described below. Some of the regulatory regions indicated below as well as additional regulatory regions are described in more detail in U.S. Patent Application Ser. Nos. 60/505,689; 60/518,075; 60/544,771; 60/558,869; 60/583,691; 60/619,181; 60/637,140; 60/757,544; 60/776,307; Ser. Nos. 10/957,569; 11/058,689; 11/172,703; 11/208,308; 11/274,890; 60/583,609; 60/612,891; 11/097,589; 11/233,726; 11/408,791; 11/414,142; 10/950,321; 11/360,017; PCT/US05/011105; PCT/US05/034308; PCT/US05/23639; PCT/US05/034308; PCT/US05/034343; and PCT/US06/038236; PCT/US06/040572; and PCT/US07/62762.

For example, the sequences of regulatory regions p326, YP0144, YP0190, p13879, YP0050, p32449, 21876, YP0158, YP0214, YP0380, PT0848, PT0633, YP0128, YP0275, PT0660, PT0683, PT0758, PT0613, PT0672, PT0688, PT0837, YP0092, PT0676, PT0708, YP0396, YP0007, YP0111, YP0103, YP0028, YP0121, YP0008, YP0039, YP0115, YP0119, YP0120, YP0374, YP0101, YP0102, YP0110, YP0117, YP0137, YP0285, YP0212, YP0097, YP0107, YP0088, YP0143, YP0156, PT0650, PT0695, PT0723, PT0838, PT0879, PT0740, PT0535, PT0668, PT0886, PT0585, YP0381, YP0337, PT0710, YP0356, YP0385, YP0384, YP0286, YP0377, PD1367, PT0863, PT0829, PT0665, PT0678, YP0086, YP0188, YP0263, PT0743 and YP0096 are set forth in the sequence listing of PCT/US06/040572; the sequence of regulatory region PT0625 is set forth in the sequence listing of PCT/US05/034343; the sequences of regulatory regions PT0623, YP0388, YP0087, YP0093, YP0108, YP0022 and YP0080 are set forth in the sequence listing of U.S. patent application Ser. No. 11/172,703; the sequence of regulatory region PR0924 is set forth in the sequence listing of PCT/US07/62762; and the sequences of regulatory regions p530c10, pOsFIE2-2, pOsMEA, pOsYp102, and pOsYp285 are set forth in the sequence listing of PCT/US06/038236. Nucleotide sequences of regulatory regions also are set forth in SEQ ID NOs:1-94 herein.

It will be appreciated that a regulatory region may meet criteria for one classification based on its activity in one plant species, and yet meet criteria for a different classification based on its activity in another plant species.

Broadly Expressing Promoters

A promoter can be said to be “broadly expressing” when it promotes transcription in many, but not necessarily all, plant tissues. For example, a broadly expressing promoter can promote transcription of an operably linked sequence in one or more of the shoot, shoot tip (apex), and leaves, but weakly or not at all in tissues such as roots or stems. As another example, a broadly expressing promoter can promote transcription of an operably linked sequence in one or more of the stem, shoot, shoot tip (apex), and leaves, but can promote transcription weakly or not at all in tissues such as reproductive tissues of flowers and developing seeds. Non-limiting examples of broadly expressing promoters that can be included in the nucleic acid constructs provided herein include the p326 (SEQ ID NO:76), YP0144 (SEQ ID NO:55), YP0190 (SEQ ID NO:59), p13879 (SEQ ID NO:75), YP0050 (SEQ ID NO:35), p32449 (SEQ ID NO:77), 21876 (SEQ ID NO:1), YP0158 (SEQ ID NO:57), YP0214 (SEQ ID NO:61), YP0380 (SEQ ID NO:70), PT0848 (SEQ ID NO:26), and PT0633 (SEQ ID NO:7) promoters. Additional examples include the cauliflower mosaic virus (CaMV) 35S promoter, the mannopine synthase (MAS) promoter, the 1′ or 2′ promoters derived from T-DNA of Agrobacterium tumefaciens, the figwort mosaic virus 34S promoter, actin promoters such as the rice actin promoter, and ubiquitin promoters such as the maize ubiquitin-1 promoter. In some cases, the CaMV 35S promoter is excluded from the category of broadly expressing promoters.

Root Promoters

Root-active promoters confer transcription in root tissue, e.g., root endodermis, root epidermis, or root vascular tissues. In some embodiments, root-active promoters are root-preferential promoters, i.e., confer transcription only or predominantly in root tissue. Root-preferential promoters include the YP0128 (SEQ ID NO:52), YP0275 (SEQ ID NO:63), PT0625 (SEQ ID NO:6), PT0660 (SEQ ID NO:9), PT0683 (SEQ ID NO:14), and PT0758 (SEQ ID NO:22) promoters. Other root-preferential promoters include the PT0613 (SEQ ID NO:5), PT0672 (SEQ ID NO:11), PT0688 (SEQ ID NO:15), and PT0837 (SEQ ID NO:24) promoters, which drive transcription primarily in root tissue and to a lesser extent in ovules and/or seeds. Other examples of root-preferential promoters include the root-specific subdomains of the CaMV 35S promoter (Lam et al., Proc. Natl. Acad. Sci. USA, 86:7890-7894 (1989)), root cell specific promoters reported by Conkling et al., Plant Physiol., 93:1203-1211 (1990), and the tobacco RD2 promoter.

Maturing Endosperm Promoters

In some embodiments, promoters that drive transcription in maturing endosperm can be useful. Transcription from a maturing endosperm promoter typically begins after fertilization and occurs primarily in endosperm tissue during seed development and is typically highest during the cellularization phase. Most suitable are promoters that are active predominantly in maturing endosperm, although promoters that are also active in other tissues can sometimes be used. Non-limiting examples of maturing endosperm promoters that can be included in the nucleic acid constructs provided herein include the napin promoter, the Arcelin-5 promoter, the phaseolin promoter (Bustos et al., Plant Cell, 1(9):839-853 (1989)), the soybean trypsin inhibitor promoter (Riggs et al., Plant Cell, 1(6):609-621 (1989)), the ACP promoter (Baerson et al., Plant Mol. Biol., 22(2):255-267 (1993)), the stearoyl-ACP desaturase promoter (Slocombe et al., Plant Physiol., 104(4):167-176 (1994)), the soybean α′ subunit of β-conglycinin promoter (Chen et al., Proc. Natl. Acad. Sci. USA, 83:8560-8564 (1986)), the oleosin promoter (Hong et al., Plant Mol. Biol., 34(3):549-555 (1997)), and zein promoters, such as the 15 kD zein promoter, the 16 kD zein promoter, 19 kD zein promoter, 22 kD zein promoter and 27 kD zein promoter. Also suitable are the Osgt-1 promoter from the rice glutelin-1 gene (Zheng et al., Mol. Cell. Biol., 13:5829-5842 (1993)), the beta-amylase promoter, and the barley hordein promoter. Other maturing endosperm promoters include the YP0092 (SEQ ID NO:38), PT0676 (SEQ ID NO:12), and PT0708 (SEQ ID NO:17) promoters.

Ovary Tissue Promoters

Promoters that are active in ovary tissues such as the ovule wall and mesocarp can also be useful, e.g., a polygalacturonidase promoter, the banana TRX promoter, the melon actin promoter, YP0396 (SEQ ID NO:74), and PT0623 (SEQ ID NO:94). Examples of promoters that are active primarily in ovules include YP0007 (SEQ ID NO:30), YP0111 (SEQ ID NO:46), YP0092 (SEQ ID NO:38), YP0103 (SEQ ID NO:43), YP0028 (SEQ ID NO:33), YP0121 (SEQ ID NO:51), YP0008 (SEQ ID NO:31), YP0039 (SEQ ID NO:34), YP0115 (SEQ ID NO:47), YP0119 (SEQ ID NO:49), YP0120 (SEQ ID NO:50), and YP0374 (SEQ ID NO:68).

Embryo Sac/Early Endosperm Promoters

To achieve expression in embryo sac/early endosperm, regulatory regions can be used that are active in polar nuclei and/or the central cell, or in precursors to polar nuclei, but not in egg cells or precursors to egg cells. Most suitable are promoters that drive expression only or predominantly in polar nuclei or precursors thereto and/or the central cell. A pattern of transcription that extends from polar nuclei into early endosperm development can also be found with embryo sac/early endosperm-preferential promoters, although transcription typically decreases significantly in later endosperm development during and after the cellularization phase. Expression in the zygote or developing embryo typically is not present with embryo sac/early endosperm promoters.

Promoters that may be suitable include those derived from the following genes: Arabidopsis viviparous-1 (see, GenBank No. U93215); Arabidopsis atmycl (see, Urao (1996) Plant Mol. Biol., 32:571-57; Conceicao (1994) Plant, 5:493-505); Arabidopsis FIE (GenBank No. AF129516); Arabidopsis MEA; Arabidopsis FIS2 (GenBank No. AF096096); and FIE 1.1 (U.S. Pat. No. 6,906,244). Other promoters that may be suitable include those derived from the following genes: maize MAC1 (see, Sheridan (1996) Genetics, 142:1009-1020); maize Cat3 (see, GenBank No. L05934; Abler (1993) Plant Mol. Biol., 22:10131-1038). Other promoters include the following Arabidopsis promoters: YP0039 (SEQ ID NO:34), YP0101 (SEQ ID NO:41), YP0102 (SEQ ID NO:42), YP0110 (SEQ ID NO:45), YP0117 (SEQ ID NO:48), YP0119 (SEQ ID NO:49), YP0137 (SEQ ID NO:53), DME, YP0285 (SEQ ID NO:64), and YP0212 (SEQ ID NO:60). Other promoters that may be useful include the following rice promoters: p530c10 (SEQ ID NO:79), pOsFIE2-2 (SEQ ID NO:80), pOsMEA (SEQ ID NO:81), pOsYp102 (SEQ ID NO:82), and pOsYp285 (SEQ ID NO:83).

Embryo Promoters

Regulatory regions that preferentially drive transcription in zygotic cells following fertilization can provide embryo-preferential expression. Most suitable are promoters that preferentially drive transcription in early stage embryos prior to the heart stage, but expression in late stage and maturing embryos is also suitable. Embryo-preferential promoters include the barley lipid transfer protein (Ltpl) promoter (Plant Cell Rep (2001) 20:647-654), YP0097 (SEQ ID NO:40), YP0107 (SEQ ID NO:44), YP0088 (SEQ ID NO:37), YP0143 (SEQ ID NO:54), YP0156 (SEQ ID NO:56), PT0650 (SEQ ID NO:8), PT0695 (SEQ ID NO:16), PT0723 (SEQ ID NO:19), PT0838 (SEQ ID NO:25), PT0879 (SEQ ID NO:28), and PT0740 (SEQ ID NO:20).

Photosynthetic Tissue Promoters

Promoters active in photosynthetic tissue confer transcription in green tissues such as leaves and stems. Most suitable are promoters that drive expression only or predominantly in such tissues. Examples of such promoters include the ribulose-1,5-bisphosphate carboxylase (RbcS) promoters such as the RbcS promoter from eastern larch (Larix laricina), the pine cab6 promoter (Yamamoto et al., Plant Cell Physiol., 35:773-778 (1994)), the Cab-1 promoter from wheat (Fejes et al., Plant Mol. Biol., 15:921-932 (1990)), the CAB-1 promoter from spinach (Lubberstedt et al., Plant Physiol., 104:997-1006 (1994)), the cab1R promoter from rice (Luan et al., Plant Cell, 4:971-981 (1992)), the pyruvate orthophosphate dikinase (PPDK) promoter from corn (Matsuoka et al., Proc. Natl. Acad. Sci. USA, 90:9586-9590 (1993)), the tobacco Lhcb1*2 promoter (Cerdan et al., Plant Mol. Biol., 33:245-255 (1997)), the Arabidopsis thaliana SUC2 sucrose-H+ symporter promoter (Truernit et al., Planta, 196:564-570 (1995)), and thylakoid membrane protein promoters from spinach (psaD, psaF, psaE, PC, FNR, atpC, atpD, cab, rbcS). Other photosynthetic tissue promoters include PT0535 (SEQ ID NO:3), PT0668 (SEQ ID NO:2), PT0886 (SEQ ID NO:29), YP0144 (SEQ ID NO:55), YP0380 (SEQ ID NO:70) and PT0585 (SEQ ID NO:4).

Vascular Tissue Promoters

Examples of promoters that have high or preferential activity in vascular bundles include YP0087 (SEQ ID NO:86), YP0093 (SEQ ID NO:87), YP0108 (SEQ ID NO:88), YP0022 (SEQ ID NO:89), and YP0080 (SEQ ID NO:90). Other vascular tissue-preferential promoters include the glycine-rich cell wall protein GRP 1.8 promoter (Keller and Baumgartner, Plant Cell, 3(10):1051-1061 (1991)), the Commelina yellow mottle virus (CoYMV) promoter (Medberry et al., Plant Cell, 4(2):185-192 (1992)), and the rice tungro bacilliform virus (RTBV) promoter (Dai et al., Proc. Natl. Acad. Sci. USA, 101(2):687-692 (2004)).

Inducible Promoters

Inducible promoters confer transcription in response to external stimuli such as chemical agents or environmental stimuli. For example, inducible promoters can confer transcription in response to hormones such as giberellic acid or ethylene, or in response to light or drought. Examples of drought-inducible promoters include YP0380 (SEQ ID NO:70), PT0848 (SEQ ID NO:26), YP0381 (SEQ ID NO:71), YP0337 (SEQ ID NO:66), PT0633 (SEQ ID NO:7), YP0374 (SEQ ID NO:68), PT0710 (SEQ ID NO:18), YP0356 (SEQ ID NO:67), YP0385 (SEQ ID NO:73), YP0396 (SEQ ID NO:74), YP0388 (SEQ ID NO:92), YP0384 (SEQ ID NO:72), PT0688 (SEQ ID NO:15), YP0286 (SEQ ID NO:65), YP0377 (SEQ ID NO:69), PD1367 (SEQ ID NO:78), and PD0901 (SEQ ID NO:93). Examples of nitrogen-inducible promoters include PT0863 (SEQ ID NO:27), PT0829 (SEQ ID NO:23), PT0665 (SEQ ID NO:10), and PT0886 (SEQ ID NO:29). Examples of shade-inducible promoters include PR0924 (SEQ ID NO:91) and PT0678 (SEQ ID NO:13).

Basal Promoters

A basal promoter is the minimal sequence necessary for assembly of a transcription complex required for transcription initiation. Basal promoters frequently include a “TATA box” element that may be located between about 15 and about 35 nucleotides upstream from the site of transcription initiation. Basal promoters also may include a “CCAAT box” element (typically the sequence CCAAT) and/or a GGGCG sequence, which can be located between about 40 and about 200 nucleotides, typically about 60 to about 120 nucleotides, upstream from the transcription start site.

Other Promoters

Other classes of promoters include, but are not limited to, shoot-preferential, callus-preferential, trichome cell-preferential, guard cell-preferential such as PT0678 (SEQ ID NO:13), tuber-preferential, parenchyma cell-preferential, and senescence-preferential promoters. Promoters designated YP0086 (SEQ ID NO:36), YP0188 (SEQ ID NO:58), YP0263 (SEQ ID NO:62), PT0758 (SEQ ID NO:22), PT0743 (SEQ ID NO:21), PT0829 (SEQ ID NO:23), YP0119 (SEQ ID NO:49), and YP0096 (SEQ ID NO:39), as described in the above-referenced patent applications, may also be useful.

Other Regulatory Regions

A 5′ untranslated region (UTR) can be included in nucleic acid constructs described herein. A 5′ UTR is transcribed, but is not translated, and lies between the start site of the transcript and the translation initiation codon and may include the +1 nucleotide. A 3′ UTR can be positioned between the translation termination codon and the end of the transcript. UTRs can have particular functions such as increasing mRNA stability or attenuating translation. Examples of 3′ UTRs include, but are not limited to, polyadenylation signals and transcription termination sequences, e.g., a nopaline synthase termination sequence.

It will be understood that more than one regulatory region may be present in a recombinant polynucleotide, e.g., introns, enhancers, upstream activation regions, transcription terminators, and inducible elements. Thus, for example, more than one regulatory region can be operably linked to the sequence of a polynucleotide encoding a protein-modulating polypeptide.

Regulatory regions, such as promoters for endogenous genes, can be obtained by chemical synthesis or by subcloning from a genomic DNA that includes such a regulatory region. A nucleic acid comprising such a regulatory region can also include flanking sequences that contain restriction enzyme sites that facilitate subsequent manipulation.

Transgenic Plants and Plant Cells

The invention also features transgenic plant cells and plants comprising at least one recombinant nucleic acid construct described herein. A plant or plant cell can be transformed by having a construct integrated into its genome, i.e., can be stably transformed. Stably transformed cells typically retain the introduced nucleic acid with each cell division. A plant or plant cell can also be transiently transformed such that the construct is not integrated into its genome. Transiently transformed cells typically lose all or some portion of the introduced nucleic acid construct with each cell division such that the introduced nucleic acid cannot be detected in daughter cells after a sufficient number of cell divisions. Both transiently transformed and stably transformed transgenic plants and plant cells can be useful in the methods described herein.

Transgenic plant cells used in methods described herein can constitute part or all of a whole plant. Such plants can be grown in a manner suitable for the species under consideration, either in a growth chamber, a greenhouse, or in a field. Transgenic plants can be bred as desired for a particular purpose, e.g., to introduce a recombinant nucleic acid into other lines, to transfer a recombinant nucleic acid to other species, or for further selection of other desirable traits. Alternatively, transgenic plants can be propagated vegetatively for those species amenable to such techniques. As used herein, a transgenic plant also refers to progeny of an initial transgenic plant. Progeny includes descendants of a particular plant or plant line. Progeny of an instant plant include seeds formed on F₁, F₂, F₃, F₄, F₅, F₆ and subsequent generation plants, or seeds formed on BC₁, BC₂, BC₃, and subsequent generation plants, or seeds formed on F₁BC₁, F₁BC₂, F₁BC₃, and subsequent generation plants. The designation F₁ refers to the progeny of a cross between two parents that are genetically distinct. The designations F₂, F₃, F₄, F₅ and F₆ refer to subsequent generations of self- or sib-pollinated progeny of an F₁ plant. Seeds produced by a transgenic plant can be grown and then selfed (or outcrossed and selfed) to obtain seeds homozygous for the nucleic acid construct.

Transgenic plants can be grown in suspension culture, or tissue or organ culture. For the purposes of this invention, solid and/or liquid tissue culture techniques can be used. When using solid medium, transgenic plant cells can be placed directly onto the medium or can be placed onto a filter that is then placed in contact with the medium. When using liquid medium, transgenic plant cells can be placed onto a flotation device, e.g., a porous membrane that contacts the liquid medium. Solid medium typically is made from liquid medium by adding agar. For example, a solid medium can be Murashige and Skoog (MS) medium containing agar and a suitable concentration of an auxin, e.g., 2,4-dichlorophenoxyacetic acid (2,4-D), and a suitable concentration of a cytokinin, e.g., kinetin.

When transiently transformed plant cells are used, a reporter sequence encoding a reporter polypeptide having a reporter activity can be included in the transformation procedure and an assay for reporter activity or expression can be performed at a suitable time after transformation. A suitable time for conducting the assay typically is about 1-21 days after transformation, e.g., about 1-14 days, about 1-7 days, or about 1-3 days. The use of transient assays is particularly convenient for rapid analysis in different species, or to confirm expression of a heterologous protein-modulating polypeptide whose expression has not previously been confirmed in particular recipient cells.

Techniques for introducing nucleic acids into monocotyledonous and dicotyledonous plants are known in the art, and include, without limitation, Agrobacterium-mediated transformation, viral vector-mediated transformation, electroporation and particle gun transformation, e.g., U.S. Pat. Nos. 5,538,880; 5,204,253; 6,329,571 and 6,013,863. If a cell or cultured tissue is used as the recipient tissue for transformation, plants can be regenerated from transformed cultures if desired, by techniques known to those skilled in the art.

In aspects related to making transgenic plants, a typical step involves selection or screening of transformed plants, e.g., for the presence of a functional vector as evidenced by expression of a selectable marker. Selection or screening can be carried out among a population of recipient cells to identify transformants using selectable marker genes such as herbicide resistance genes. Physical and biochemical methods can be used to identify transformants. These include Southern analysis or PCR amplification for detection of a polynucleotide; Northern blots, S1 RNase protection, primer-extension, or RT-PCR amplification for detecting RNA transcripts; enzymatic assays for detecting enzyme or ribozyme activity of polypeptides and polynucleotides; and protein gel electrophoresis, Western blots, immunoprecipitation, and enzyme-linked immunoassays to detect polypeptides. Other techniques such as in situ hybridization, enzyme staining, and immunostaining also can be used to detect the presence or expression of polypeptides and/or polynucleotides. Methods for performing all of the referenced techniques are known.

A population of transgenic plants can be screened and/or selected for those members of the population that have a desired trait or phenotype conferred by expression of the transgene. For example, a population of progeny of a single transformation event can be screened for those plants having a desired level of expression of a heterologous protein-modulating polypeptide or nucleic acid. As an alternative, a population of plants comprising independent transformation events can be screened for those plants having a desired trait, such as a modulated level of protein. Selection and/or screening can be carried out over one or more generations, which can be useful to identify those plants that have a statistically significant difference in a protein level as compared to a corresponding level in a control plant. Selection and/or screening can also be carried out in more than one geographic location. In some cases, transgenic plants can be grown and selected under conditions which induce a desired phenotype or are otherwise necessary to produce a desired phenotype in a transgenic plant. In addition, selection and/or screening can be carried out during a particular developmental stage in which the phenotype is expected to be exhibited by the plant. Selection and/or screening can be carried out to choose those transgenic plants having a statistically significant difference in a protein level relative to a control plant that lacks the transgene. Selected or screened transgenic plants have an altered phenotype as compared to a corresponding control plant, as described in the “Transgenic Plant Phenotypes” section below.

Plant Species

The polynucleotides and vectors described herein can be used to transform a number of monocotyledonous and dicotyledonous plants and plant cell systems, including dicots such as alfalfa, almond, amaranth, apple, beans (including kidney beans, lima beans, dry beans, green beans), brazil nut, broccoli, cabbage, carrot, cashew, castor bean, cherry, chick peas, chicory, clover, cocoa, coffee, cotton, crambe, flax, grape, grapefruit, hazelnut, lemon, lentils, lettuce, linseed, macadamia nut, mango, melon (e.g., watermelon, cantaloupe), mustard, orange, peach, peanut, pear, peas, pecan, pepper, pistachio, plum, potato, oilseed rape, quinoa, rapeseed (high erucic acid and canola), safflower, sesame, soybean, spinach, strawberry, sugar beet, sunflower, sweet potatoes, tea, tomato, walnut, and yams, as well as monocots such as banana, barley, bluegrass, date palm, fescue, field corn, garlic, millet, oat, oil palm, onion, pineapple, popcorn, rice, rye, ryegrass, sorghum, sudangrass, sugarcane, sweet corn, switchgrass, timothy, and wheat. Brown seaweeds, green seaweeds, red seaweeds, and microalgae can also be used.

Thus, the methods and compositions described herein can be used with dicotyledonous plants belonging, for example, to the orders Apiales, Arecales, Aristochiales, Asterales, Batales, Campanulales, Capparales, Caryophyllales, Casuarinales, Celastrales, Cornales, Cucurbitales, Diapensales, Dilleniales, Dipsacales, Ebenales, Ericales, Eucomiales, Euphorbiales, Fabales, Fagales, Gentianales, Geraniales, Haloragales, Hamamelidales, Illiciales, Juglandales, Lamiales, Laurales, Lecythidales, Leitneriales, Linales, Magniolales, Malvales, Myricales, Myrtales, Nymphaeales, Papaverales, Piperales, Plantaginales, Plumbaginales, Podostemales, Polemoniales, Polygalales, Polygonales, Primulales, Proteales, Rafflesiales, Ranunculales, Rhamnales, Rosales, Rubiales, Salicales, Santales, Sapindales, Sarraceniaceae, Scrophulariales, Solanales, Trochodendrales, Theales, Umbellales, Urticales, and Violales. The methods and compositions described herein also can be utilized with monocotyledonous plants such as those belonging to the orders Alismatales, Arales, Arecales, Asparagales, Bromeliales, Commelinales, Cyclanthales, Cyperales, Eriocaulales, Hydrocharitales, Juncales, Liliales, Najadales, Orchidales, Pandanales, Poales, Restionales, Triuridales, Typhales, Zingiberales, and with plants belonging to Gymnospermae, e.g., Cycadales, Ginkgoales, Gnetales, and Pinales.

The methods and compositions can be used over a broad range of plant species, including species from the dicot genera Amaranthus, Anacardium, Arachis, Bertholletia, Brassica, Calendula, Camellia, Capsicum, Carthamus, Carya, Chenopodium, Cicer, Cichorium, Cinnamomum, Citrus, Citrullus, Coffea, Corylus, Crambe, Cucumis, Cucurbita, Daucus, Dioscorea, Fragaria, Glycine, Gossypium, Helianthus, Juglans, Lactuca, Lens, Linum, Lycopersicon, Macadamia, Malus, Mangifera, Medicago, Mentha, Nicotiana, Ocimum, Olea, Phaseolus, Pistacia, Pisum, Prunus, Pyrus, Rosmarinus, Salvia, Sesamum, Solanum, Spinacia, Theobroma, Thymus, Trifolium, Vaccinium, Vigna, and Vitis; and the monocot genera Allium, Ananas, Asparagus, Avena, Curcuma, Elaeis, Festuca, Hordeum, Lemna, Lolium, Musa, Oryza, Panicum, Pennisetum, Phleum, Poa, Saccharum, Secale, Sorghum, Triticosecale, Triticum, and Zea.

The methods and compositions described herein also can be used with brown seaweeds, e.g., Ascophyllum nodosum, Fucus vesiculosus, Fucus serratus, Himanthalia elongata, and Undaria pinnatifida; red seaweeds, e.g., Chondrus crispus, Cracilaria verrucosa, Porphyra umbilicalis, and Palmaria palmata; green seaweeds, e.g., Enteromorpha spp. and Ulva spp.; and microalgae, e.g., Spirulina spp. (S. platensis and S. maxima) and Odontella aurita. In addition, the methods and compositions can be used with Crypthecodinium cohnii, Schizochytrium spp., and Haematococcus pluvialis.

In some embodiments, a plant is a member of the species Avena sativa, Brassica spp., Cicer arietinum, Gossypium spp., Glycine max, Hordeum vulgare, Lactuca sativa, Medicago sativa, Oryza sativa, Pennisetum glaucum, Phaseolus spp., Phleum pratense, Secale cereale, Trifolium pratense, Triticum aestivum, and Zea mays.

Expression of Protein-Modulating Polypeptides

The polynucleotides and recombinant vectors described herein can be used to express a protein-modulating polypeptide in a plant species of interest. The term “expression” refers to the process of converting genetic information of a polynucleotide into RNA through transcription, which is catalyzed by an enzyme, RNA polymerase, and into protein, through translation of mRNA on ribosomes. “Up-regulation” or “activation” refers to regulation that increases the production of expression products (mRNA, polypeptide, or both) relative to basal or native states, while “down-regulation” or “repression” refers to regulation that decreases production of expression products (mRNA, polypeptide, or both) relative to basal or native states.

In some cases, expression of a protein-modulating polypeptide inhibits one or more functions of an endogenous polypeptide. For example, a nucleic acid that encodes a dominant negative polypeptide can be used to inhibit protein function. A dominant negative polypeptide typically is mutated or truncated relative to an endogenous wild type polypeptide, and its presence in a cell inhibits one or more functions of the wild type polypeptide in that cell, i.e., the dominant negative polypeptide is genetically dominant and confers a loss of function. The mechanism by which a dominant negative polypeptide confers such a phenotype can vary but often involves a protein-protein interaction or a protein-DNA interaction. For example, a dominant negative polypeptide can be an enzyme that is truncated relative to a native wild type enzyme, such that the truncated polypeptide retains domains involved in binding a first protein but lacks domains involved in binding a second protein. The truncated polypeptide is thus unable to properly modulate the activity of the second protein. See, e.g., US 2007/0056058. As another example, a point mutation that results in a non-conservative amino acid substitution in a catalytic domain can result in a dominant negative polypeptide. See, e.g., US 2005/032221. As another example, a dominant negative polypeptide can be a transcription factor that is truncated relative to a native wild type transcription factor, such that the truncated polypeptide retains the DNA binding domain(s) but lacks the activation domain(s). Such a truncated polypeptide can inhibit the wild type transcription factor from binding DNA, thereby inhibiting transcription activation.

A number of nucleic acid based methods, including antisense RNA, ribozyme directed RNA cleavage, post-transcriptional gene silencing (PTGS), e.g., RNA interference (RNAi), and transcriptional gene silencing (TGS) can be used to inhibit gene expression in plants. Antisense technology is one well-known method. In this method, a nucleic acid segment from a gene to be repressed is cloned and operably linked to a regulatory region and a transcription termination sequence so that the antisense strand of RNA is transcribed. The recombinant vector is then transformed into plants, as described herein, and the antisense strand of RNA is produced. The nucleic acid segment need not be the entire sequence of the gene to be repressed, but typically will be substantially complementary to at least a portion of the sense strand of the gene to be repressed. Generally, higher homology can be used to compensate for the use of a shorter sequence. Typically, a sequence of at least 30 nucleotides is used, e.g., at least 40, 50, 80, 100, 200, 500 nucleotides or more.

In another method, a nucleic acid can be transcribed into a ribozyme, or catalytic RNA, that affects expression of an mRNA. See, U.S. Pat. No. 6,423,885. Ribozymes can be designed to specifically pair with virtually any target RNA and cleave the phosphodiester backbone at a specific location, thereby functionally inactivating the target RNA. Heterologous nucleic acids can encode ribozymes designed to cleave particular mRNA transcripts, thus preventing expression of a polypeptide. Hammerhead ribozymes are useful for destroying particular mRNAs, although various ribozymes that cleave mRNA at site-specific recognition sequences can be used. Hammerhead ribozymes cleave mRNAs at locations dictated by flanking regions that form complementary base pairs with the target mRNA. The sole requirement is that the target RNA contain a 5′-UG-3′ nucleotide sequence. The construction and production of hammerhead ribozymes is known in the art. See, for example, U.S. Pat. No. 5,254,678 and WO 02/46449 and references cited therein. Hammerhead ribozyme sequences can be embedded in a stable RNA such as a transfer RNA (tRNA) to increase cleavage efficiency in vivo. Perriman et al., Proc. Natl. Acad. Sci. USA, 92(13):6175-6179 (1995); de Feyter and Gaudron, Methods in Molecular Biology, Vol. 74, Chapter 43, “Expressing Ribozymes in Plants”, Edited by Turner, P. C., Humana Press Inc., Totowa, N.J. RNA endoribonucleases which have been described, such as the one that occurs naturally in Tetrahymena thermophile, can be useful. See, for example, U.S. Pat. Nos. 4,987,071 and 6,423,885.

PTGS, e.g., RNAi, can also be used to inhibit the expression of a gene. For example, a construct can be prepared that includes a sequence that is transcribed into an RNA that can anneal to itself, e.g., a double stranded RNA having a stem-loop structure. In some embodiments, one strand of the stem portion of a double stranded RNA comprises a sequence that is similar or identical to the sense coding sequence of a protein-modulating polypeptide, and that is from about 10 nucleotides to about 2,500 nucleotides in length. The length of the sequence that is similar or identical to the sense coding sequence can be from 10 nucleotides to 500 nucleotides, from 15 nucleotides to 300 nucleotides, from 20 nucleotides to 100 nucleotides, or from 25 nucleotides to 100 nucleotides. The other strand of the stem portion of a double stranded RNA comprises a sequence that is similar or identical to the antisense strand of the coding sequence of the protein-modulating polypeptide, and can have a length that is shorter, the same as, or longer than the corresponding length of the sense sequence. In some cases, one strand of the stem portion of a double stranded RNA comprises a sequence that is similar or identical to the 3′ or 5′ untranslated region of an mRNA encoding a protein-modulating polypeptide, and the other strand of the stem portion of the double stranded RNA comprises a sequence that is similar or identical to the sequence that is complementary to the 3′ or 5′ untranslated region, respectively, of the mRNA encoding the protein-modulating polypeptide. In other embodiments, one strand of the stem portion of a double stranded RNA comprises a sequence that is similar or identical to the sequence of an intron in the pre-mRNA encoding a protein-modulating polypeptide, and the other strand of the stem portion comprises a sequence that is similar or identical to the sequence that is complementary to the sequence of the intron in the pre-mRNA. The loop portion of a double stranded RNA can be from 3 nucleotides to 5,000 nucleotides, e.g., from 3 nucleotides to 25 nucleotides, from 15 nucleotides to 1,000 nucleotides, from 20 nucleotides to 500 nucleotides, or from 25 nucleotides to 200 nucleotides. The loop portion of the RNA can include an intron. A double stranded RNA can have zero, one, two, three, four, five, six, seven, eight, nine, ten, or more stem-loop structures. A construct including a sequence that is operably linked to a regulatory region and a transcription termination sequence, and that is transcribed into an RNA that can form a double stranded RNA, is transformed into plants as described herein. Methods for using RNAi to inhibit the expression of a gene are known to those of skill in the art. See, e.g., U.S. Pat. Nos. 5,034,323; 6,326,527; 6,452,067; 6,573,099; 6,753,139; and 6,777,588. See also WO 97/01952; WO 98/53083; WO 99/32619; WO 98/36083; and U.S. Patent Publications 20030175965, 20030175783, 20040214330, and 20030180945.

Constructs containing regulatory regions operably linked to nucleic acid molecules in sense orientation can also be used to inhibit the expression of a gene. The transcription product can be similar or identical to the sense coding sequence of a protein-modulating polypeptide. The transcription product can also be unpolyadenylated, lack a 5′ cap structure, or contain an unsplicable intron. Methods of inhibiting gene expression using a full-length cDNA as well as a partial cDNA sequence are known in the art. See, e.g., U.S. Pat. No. 5,231,020.

In some embodiments, a construct containing a nucleic acid having at least one strand that is a template for both sense and antisense sequences that are complementary to each other is used to inhibit the expression of a gene. The sense and antisense sequences can be part of a larger nucleic acid molecule or can be part of separate nucleic acid molecules having sequences that are not complementary. The sense or antisense sequence can be a sequence that is identical or complementary to the sequence of an mRNA, the 3′ or 5′ untranslated region of an mRNA, or an intron in a pre-mRNA encoding a protein-modulating polypeptide. In some embodiments, the sense or antisense sequence is identical or complementary to a sequence of the regulatory region that drives transcription of the gene encoding a protein-modulating polypeptide. In each case, the sense sequence is the sequence that is complementary to the antisense sequence.

The sense and antisense sequences can be any length greater than about 10 nucleotides (e.g., 12, 13, 14, 15, 16, 17, 18, 19, 20, 21, 22, 23, 24, 25, 26, 27, 28, 29, 30, or more nucleotides). For example, an antisense sequence can be 21 or 22 nucleotides in length. Typically, the sense and antisense sequences range in length from about 15 nucleotides to about 30 nucleotides, e.g., from about 18 nucleotides to about 28 nucleotides, or from about 21 nucleotides to about 25 nucleotides.

In some embodiments, an antisense sequence is a sequence complementary to an mRNA sequence encoding a protein-modulating polypeptide described herein. The sense sequence complementary to the antisense sequence can be a sequence present within the mRNA of the protein-modulating polypeptide. Typically, sense and antisense sequences are designed to correspond to a 15-30 nucleotide sequence of a target mRNA such that the level of that target mRNA is reduced.

In some embodiments, a construct containing a nucleic acid having at least one strand that is a template for more than one sense sequence (e.g., 2, 3, 4, 5, 6, 7, 8, 9, 10 or more sense sequences) can be used to inhibit the expression of a gene. Likewise, a construct containing a nucleic acid having at least one strand that is a template for more than one antisense sequence (e.g., 2, 3, 4, 5, 6, 7, 8, 9, 10 or more antisense sequences) can be used to inhibit the expression of a gene. For example, a construct can contain a nucleic acid having at least one strand that is a template for two sense sequences and two antisense sequences. The multiple sense sequences can be identical or different, and the multiple antisense sequences can be identical or different. For example, a construct can have a nucleic acid having one strand that is a template for two identical sense sequences and two identical antisense sequences that are complementary to the two identical sense sequences. Alternatively, an isolated nucleic acid can have one strand that is a template for (1) two identical sense sequences 20 nucleotides in length, (2) one antisense sequence that is complementary to the two identical sense sequences 20 nucleotides in length, (3) a sense sequence 30 nucleotides in length, and (4) three identical antisense sequences that are complementary to the sense sequence 30 nucleotides in length. The constructs provided herein can be designed to have any arrangement of sense and antisense sequences. For example, two identical sense sequences can be followed by two identical antisense sequences or can be positioned between two identical antisense sequences.

A nucleic acid having at least one strand that is a template for one or more sense and/or antisense sequences can be operably linked to a regulatory region to drive transcription of an RNA molecule containing the sense and/or antisense sequence(s). In addition, such a nucleic acid can be operably linked to a transcription terminator sequence, such as the terminator of the nopaline synthase (nos) gene. In some cases, two regulatory regions can direct transcription of two transcripts: one from the top strand, and one from the bottom strand. See, for example, Yan et al., Plant Physiol., 141:1508-1518 (2006). The two regulatory regions can be the same or different. The two transcripts can form double-stranded RNA molecules that induce degradation of the target RNA. In some cases, a nucleic acid can be positioned within a T-DNA or P-DNA such that the left and right T-DNA border sequences, or the left and right border-like sequences of the P-DNA, flank or are on either side of the nucleic acid. The nucleic acid sequence between the two regulatory regions can be from about 15 to about 300 nucleotides in length. In some embodiments, the nucleic acid sequence between the two regulatory regions is from about 15 to about 200 nucleotides in length, from about 15 to about 100 nucleotides in length, from about 15 to about 50 nucleotides in length, from about 18 to about 50 nucleotides in length, from about 18 to about 40 nucleotides in length, from about 18 to about 30 nucleotides in length, or from about 18 to about 25 nucleotides in length.

In some nucleic-acid based methods for inhibition of gene expression in plants, a suitable nucleic acid can be a nucleic acid analog. Nucleic acid analogs can be modified at the base moiety, sugar moiety, or phosphate backbone to improve, for example, stability, hybridization, or solubility of the nucleic acid. Modifications at the base moiety include deoxyuridine for deoxythymidine, and 5-methyl-2′-deoxycytidine and 5-bromo-2′-deoxycytidine for deoxycytidine. Modifications of the sugar moiety include modification of the 2′ hydroxyl of the ribose sugar to form 2′-O-methyl or 2′-O-allyl sugars. The deoxyribose phosphate backbone can be modified to produce morpholino nucleic acids, in which each base moiety is linked to a six-membered morpholino ring, or peptide nucleic acids, in which the deoxyphosphate backbone is replaced by a pseudopeptide backbone and the four bases are retained. See, for example, Summerton and Weller, 1997, Antisense Nucleic Acid Drug Dev., 7:187-195; Hyrup et al., Bioorgan. Med. Chem., 4:5-23 (1996). In addition, the deoxyphosphate backbone can be replaced with, for example, a phosphorothioate or phosphorodithioate backbone, a phosphoroamidite, or an alkyl phosphotriester backbone.

Transgenic Plant Phenotypes

In some embodiments, a plant in which expression of a protein-modulating polypeptide is modulated can have increased levels of seed protein. For example, a protein-modulating polypeptide described herein can be expressed in a transgenic plant, resulting in increased levels of seed protein. The seed protein level can be increased by at least 2 percent, e.g., 2, 3, 4, 5, 6, 7, 8, 9, 10, 11, 12, 13, 14, 15, 16, 17, 18, 19, 20, 25, 30, 35, 40, 45, 50, 55, 60, or more than 60 percent, as compared to the seed protein level in a corresponding control plant that does not express the transgene. In some embodiments, a plant in which expression of a protein-modulating polypeptide is modulated can have decreased levels of seed protein. The seed protein level can be decreased by at least 2 percent, e.g., 2, 3, 4, 5, 10, 15, 20, 25, 30, 35, or more than 35 percent, as compared to the seed protein level in a corresponding control plant that does not express the transgene.

Plants for which modulation of levels of seed protein can be useful include, without limitation, amaranth, barley, beans, canola, coffee, cotton, edible nuts (e.g., almond, brazil nut, cashew, hazelnut, macadamia nut, peanut, pecan, pine nut, pistachio, walnut), field corn, millet, oat, oil palm, peas, popcorn, rapeseed, rice, rye, safflower, sorghum, soybean, sunflower, sweet corn, and wheat. Increases in seed protein in such plants can provide improved nutritional content in geographic locales where dietary intake of protein/amino acid is often insufficient. Decreases in seed protein in such plants can be useful in situations where seeds are not the primary plant part that is harvested for human or animal consumption.

In some embodiments, a plant in which expression of a protein-modulating polypeptide is modulated can have increased or decreased levels of protein in one or more non-seed tissues, e.g., leaf tissues, stem tissues, root or corm tissues, or fruit tissues other than seed. For example, the protein level can be increased by at least 2 percent, e.g., 2, 3, 4, 5, 6, 7, 8, 9, 10, 11, 12, 13, 14, 15, 16, 17, 18, 19, 20, 25, 30, 35, 40, 45, 50, 55, 60, or more than 60 percent, as compared to the protein level in a corresponding control plant that does not express the transgene. In some embodiments, a plant in which expression of a protein-modulating polypeptide is modulated can have decreased levels of protein in one or more non-seed tissues. The protein level can be decreased by at least 2 percent, e.g., 2, 3, 4, 5, 10, 15, 20, 25, 30, 35, or more than 35 percent, as compared to the protein level in a corresponding control plant that does not express the transgene.

Plants for which modulation of levels of protein in non-seed tissues can be useful include, without limitation, alfalfa, amaranth, apple, banana, barley, beans, bluegrass, broccoli, carrot, cherry, clover, coffee, fescue, field corn, grape, grapefruit, lemon, lettuce, mango, melon, millet, oat, oil palm, onion, orange, peach, peanut, pear, peas, pineapple, plum, popcorn, potato, rapeseed, rice, rye, ryegrass, safflower, sorghum, soybean, strawberry, sugarcane, sudangrass, sunflower, sweet corn, switchgrass, timothy, tomato, and wheat. Increases in non-seed protein in such plants can provide improved nutritional content in edible fruits and vegetables, or improved animal forage. Decreases in non-seed protein can provide more efficient partitioning of nitrogen to plant part(s) that are harvested for human or animal consumption.

In some embodiments, a plant in which expression of a protein-modulating polypeptide having an amino acid sequence corresponding to SEQ ID NO:102 is modulated can have modulated levels of seed oil accompanying increased levels of seed protein. The oil level can be modulated by at least 2 percent, e.g., 2, 3, 4, 5, 10, 15, 20, 25, 30, 35, or more than 35 percent.

In some embodiments, a plant in which expression of a protein-modulating polypeptide having an amino acid sequence corresponding to SEQ ID NO:96, SEQ ID NO:112, SEQ ID NO:114, or SEQ ID NO:118 is modulated can have decreased levels of seed oil accompanying increased levels of seed protein. The oil level can be decreased by at least 2 percent, e.g., 3, 4, 5, 10, 15, 20, 25, 30, 35, or more than 35 percent, as compared to the oil level in a corresponding control plant that does not express the transgene.

Typically, a difference (e.g., an increase) in the amount of oil or protein in a transgenic plant or cell relative to a control plant or cell is considered statistically significant at p≦0.05 with an appropriate parametric or non-parametric statistic, e.g., Chi-square test, Student's t-test, Mann-Whitney test, or F-test. In some embodiments, a difference in the amount of oil or protein is statistically significant at p<0.01, p<0.005, or p<0.001. A statistically significant difference in, for example, the amount of protein in a transgenic plant compared to the amount in cells of a control plant indicates that (1) the recombinant nucleic acid present in the transgenic plant results in altered protein levels and/or (2) the recombinant nucleic acid warrants further study as a candidate for altering the amount of protein in a plant.

The phenotype of a transgenic plant is evaluated relative to a control plant that does not express the exogenous polynucleotide of interest, such as a corresponding wild type plant, a corresponding plant that is not transgenic for the exogenous polynucleotide of interest but otherwise is of the same genetic background as the transgenic plant of interest, or a corresponding plant of the same genetic background in which expression of the polypeptide is suppressed, inhibited, or not induced (e.g., where expression is under the control of an inducible promoter). A plant is said “not to express” a polypeptide when the plant exhibits less than 10%, e.g., less than 9%, 8%, 7%, 6%, 5%, 4%, 3%, 2%, 1%, 0.5%, 0.1%, 0.01%, or 0.001%, of the amount of polypeptide or mRNA encoding the polypeptide exhibited by the plant of interest. Expression can be evaluated using methods including, for example, RT-PCR, Northern blots, S1 RNase protection, primer extensions, Western blots, protein gel electrophoresis, immunoprecipitation, enzyme-linked immunoassays, chip assays, and mass spectrometry. It should be noted that if a polypeptide is expressed under the control of a tissue-preferential or broadly expressing promoter, expression can be evaluated in the entire plant or in a selected tissue. Similarly, if a polypeptide is expressed at a particular time, e.g., at a particular time in development or upon induction, expression can be evaluated selectively at a desired time period.

Information that the polypeptides disclosed herein can modulate protein content can be useful in breeding of crop plants. Based on the effect of disclosed polypeptides on protein content, one can search for and identify polymorphisms linked to genetic loci for such polypeptides. Polymorphisms that can be identified include simple sequence repeats (SSRs), rapid amplification of polymorphic DNA (RAPDs), amplified fragment length polymorphisms (AFLPs) and restriction fragment length polymorphisms (RFLPs).

If a polymorphism is identified, its presence and frequency in populations is analyzed to determine if it is statistically significantly correlated to an alteration in protein content. Those polymorphisms that are correlated with an alteration in protein content can be incorporated into a marker assisted breeding program to facilitate the development of lines that have a desired alteration in protein content. Typically, a polymorphism identified in such a manner is used with polymorphisms at other loci that are also correlated with a desired alteration in protein content.

Articles of Manufacture

Transgenic plants provided herein have particular uses in the agricultural and nutritional industries. For example, transgenic plants described herein can be used to make animal feed and food products, such as grains and fresh, canned, and frozen vegetables. Suitable plants with which to make such products include alfalfa, barley, beans, clover, corn, millet, oat, peas, rice, rye, soybean, timothy, and wheat. For example, soybeans can be used to make various food products, including tofu, soy flour, and soy protein concentrates and isolates. Soy protein concentrates can be used to make textured soy protein products that resemble meat products. Soy protein isolates can be added to many soy food products, such as soy sausage patties, soybean burgers, soy protein bars, powdered soy protein beverages, soy protein baby formulas, and soy protein supplements. Such products are useful to provide increased or decreased protein and caloric content in the diet.

Seeds from transgenic plants described herein can be used as is, e.g., to grow plants, or can be used to make food products, such as flour. Seeds can be conditioned and bagged in packaging material by means known in the art to form an article of manufacture. Packaging material such as paper and cloth are well known in the art. A package of seed can have a label e.g., a tag or label secured to the packaging material, a label printed on the packaging material, or a label inserted within the package.

The invention will be further described in the following examples, which do not limit the scope of the invention described in the claims.

### EXAMPLES

### Example 1

### Transgenic Plants

The following symbols are used in the Examples: T₁: first generation transformant; T₂: second generation, progeny of self-pollinated T₁ plants; T₃: third generation, progeny of self-pollinated T₂ plants; T₄: fourth generation, progeny of self-pollinated T₃ plants. Independent transformations are referred to as events.

The following is a list of nucleic acids that were isolated from Arabidopsis thaliana plants. ANNOT ID 826303 (SEQ ID NO:95) is a DNA clone that is predicted to encode a 303 amino acid polypeptide (SEQ ID NO:96). ANNOT ID 842015 (SEQ ID NO:111) is a DNA clone that is predicted to encode a 462 amino acid RASPBERRY3 polypeptide (SEQ ID NO:112). CLONE ID 97982 (SEQ ID NO:113) is a DNA clone that is predicted to encode a 277 amino acid carbonate dehydratase-like polypeptide (SEQ ID NO:114). ANNOT ID 571199 (SEQ ID NO:101) is a DNA clone that is predicted to encode a 335 amino acid polypeptide (SEQ ID NO:102). ANNOT ID 564367 (SEQ ID NO:117) is a DNA clone that is predicted to encode a 174 amino acid heat shock polypeptide (SEQ ID NO:118). ANNOT ID 851745 (SEQ ID NO:127) is a DNA clone that is predicted to encode a 306 amino acid polypeptide (SEQ ID NO:128).

The following nucleic acid was isolated from Zea mays. 3′-truncated CLONE ID 258034 (SEQ ID NO:115) is a DNA clone that is predicted to encode a 68 amino acid polypeptide (SEQ ID NO:116). As discussed above, the polypeptide having the amino acid sequence set forth in SEQ ID NO:116 is a chimeric polypeptide. Residues 1-45 of SEQ ID NO:116 correspond to residues 1-45 of SEQ ID NO:181 while residues 46-68 of SEQ ID NO:116 correspond to the predicted read-through translational product of vector sequence.

Each isolated nucleic acid described above was cloned into a Ti plasmid vector, CRS 338, containing a phosphinothricin acetyltransferase gene which confers Finale™ resistance to transformed plants. Constructs were made using CRS 338 that contained ANNOT ID 826303, ANNOT ID 842015, CLONE ID 97982, ANNOT ID 571199, ANNOT ID 564367, ANNOT ID 851745, or 3′-truncated CLONE ID 258034, each operably linked to a CaMV 35S promoter. Wild-type Arabidopsis thaliana ecotype Wassilewskija (Ws) plants were transformed separately with each construct. The transformations were performed essentially as described in Bechtold et al., C.R. Acad. Sci. Paris, 316:1194-1199 (1993).

Transgenic Arabidopsis lines containing ANNOT ID 826303, ANNOT ID 842015, CLONE ID 97982, ANNOT ID 571199, ANNOT ID 564367, ANNOT ID 851745, or CLONE ID 258034 were designated ME11370, ME 11410, ME02482, ME11409, ME10870, ME11351, or ME07978, respectively. The presence of each vector containing a Ceres clone described above in the respective transgenic Arabidopsis line transformed with the vector was confirmed by Finale™ resistance, polymerase chain reaction (PCR) amplification from green leaf tissue extract, and/or sequencing of PCR products. As controls, wild-type Arabidopsis ecotype Ws plants were transformed with the empty vector CRS 338.

### Example 2

### Analysis of Protein Content in Transgenic Arabidopsis Seeds

An analytical method based on Fourier transform near-infrared (FT-NIR) spectroscopy was developed, validated, and used to perform a high-throughput screen of transgenic seed lines for alterations in seed protein content. To calibrate the FT-NIR spectroscopy method, total nitrogen elemental analysis was used as a primary method to analyze a sub-population of randomly selected transgenic seed lines. The overall percentage of nitrogen in each sample was determined. Percent nitrogen values were multiplied by a conversion factor to obtain percent total protein values. A conversion factor of 5.30 was selected based on data for cotton, sunflower, safflower, and sesame seed (Rhee, K. C., Determination of Total Nitrogen In Handbook of Food Analytical Chemistry—Water, Proteins, Enzymes, Lipids, and Carbohydrates (R. Wrolstad, et al., ed.), John Wiley and Sons, Inc., p. 105, (2005)). The same seed lines were then analyzed by FT-NIR spectroscopy, and the protein values calculated via the primary method were entered into the FT-NIR chemometrics software (Bruker Optics, Billerica, Mass.) to create a calibration curve for analysis of seed protein content by FT-NIR spectroscopy.

Elemental analysis was performed using a FlashEA 1112 NC Analyzer (Thermo Finnigan, San Jose, Calif.). To analyze total nitrogen content, 2.00±0.15 mg of dried transgenic Arabidopsis seed was weighed into a tared tin cup. The tin cup with the seed was weighed, crushed, folded in half, and placed into an autosampler slot on the FlashEA 1112 NC Analyzer (Thermo Finnigan). Matched controls were prepared in a manner identical to the experimental samples and spaced evenly throughout the batch. The first three samples in every batch were a blank (empty tin cup), a bypass, (approximately 5 mg of aspartic acid), and a standard (5.00±0.15 mg aspartic acid), respectively. Blanks were entered between every 15 experimental samples. Each sample was analyzed in triplicate.

The FlashEA 1112 NC Analyzer (Thermo Finnigan) instrument parameters were as follows: left furnace 900° C., right furnace 840° C., oven 50° C., gas flow carrier 130 mL/min., and gas flow reference 100 mL/min. The data parameter LLOD was 0.25 mg for the standard and different for other materials. The data parameter LLOQ was 3.0 mg for the standard, 1.0 mg for seed tissue, and different for other materials.

Quantification was performed using the Eager 300 software (Thermo Finnigan). Replicate percent nitrogen measurements were averaged and multiplied by a conversion factor of 5.30 to obtain percent total protein values. For results to be considered valid, the standard deviation between replicate samples was required to be less than 10%. The percent nitrogen of the aspartic acid standard was required to be within ±1.0% of the theoretical value. For a run to be declared valid, the weight of the aspartic acid (standard) was required to be between 4.85 and 5.15 mg, and the blank(s) were required to have no recorded nitrogen content.

The same seed lines that were analyzed for elemental nitrogen content were also analyzed by FT-NIR spectroscopy, and the percent total protein values determined by elemental analysis were entered into the FT-NIR chemometrics software (Bruker Optics, Billerica, Mass.) to create a calibration curve for protein content. The protein content of each seed line based on total nitrogen elemental analysis was plotted on the x-axis of the calibration curve. The y-axis of the calibration curve represented the predicted values based on the best-fit line. Data points were continually added to the calibration curve data set.

T₂ seed from each transgenic plant line was analyzed by FT-NIR spectroscopy. Sarstedt tubes containing seeds were placed directly on the lamp, and spectra were acquired through the bottom of the tube. The spectra were analyzed to determine seed protein content using the FT-NIR chemometrics software (Bruker Optics) and the protein calibration curve. Results for experimental samples were compared to population means and standard deviations calculated for transgenic seed lines that were planted within 30 days of the lines being analyzed and grown under the same conditions. Typically, results from three to four events of each of 400 to 1600 different transgenic lines were used to calculate a population mean. Each data point was assigned a z-score (z=(x−mean)/std), and a p-value was calculated for the z-score.

Transgenic seed lines with protein levels in T₂ seed that differed by more than two standard deviations from the population mean were selected for evaluation of protein levels in the T₃ generation. All events of selected lines were planted in individual pots. The pots were arranged randomly in flats along with pots containing matched control plants in order to minimize microenvironment effects. Matched control plants contained an empty version of the vector used to generate the transgenic seed lines. T₃ seed from up to five plants from each event was collected and analyzed individually using FT-NIR spectroscopy. Data from replicate samples were averaged and compared to controls using the Student's t-test.

### Example 3

### Analysis of Oil Content in Transgenic Arabidopsis Seeds

An analytical method based on Fourier transform near-infrared (FT-NIR) spectroscopy was developed, validated, and used to perform a high-throughput screen of transgenic seed lines for alterations in seed oil content. To calibrate the FT-NIR spectroscopy method, a sub-population of transgenic seed lines was randomly selected and analyzed for oil content using a direct primary method. Fatty acid methyl ester (FAME) analysis by gas chromatography-mass spectroscopy (GC-MS) was used as the direct primary method to determine the total fatty acid content for each seed line and produce the FT-NIR spectroscopy calibration curves for oil.

To analyze seed oil content using GC-MS, seed tissue was homogenized in liquid nitrogen using a mortar and pestle to create a powder. The tissue was weighed, and 5.0±0.25 mg were transferred into a 2 mL Eppendorf tube. The exact weight of each sample was recorded. One mL of 2.5% H₂SO₄ (v/v in methanol) and 20 μL of undecanoic acid internal standard (1 mg/mL in hexane) were added to the weighed seed tissue. The tubes were incubated for two hours at 90° C. in a pre-equilibrated heating block. The samples were removed from the heating block and allowed to cool to room temperature. The contents of each Eppendorf tube were poured into a 15 mL polypropylene conical tube, and 1.5 ml, of a 0.9% NaCl solution and 0.75 mL of hexane were added to each tube. The tubes were vortexed for 30 seconds and incubated at room temperature for 15 minutes. The samples were then centrifuged at 4,000 rpm for 5 minutes using a bench top centrifuge. If emulsions remained, then the centrifugation step was repeated until they were dissipated. One hundred μL of the hexane (top) layer was pipetted into a 1.5 mL autosampler vial with minimum volume insert. The samples were stored no longer than 1 week at −80° C. until they were analyzed.

Samples were analyzed using a Shimadzu QP-2010 GC-MS (Shimadzu Scientific Instruments, Columbia, Md.). The first and last sample of each batch consisted of a blank (hexane). Every fifth sample in the batch also consisted of a blank. Prior to sample analysis, a 7-point calibration curve was generated using the Supelco 37 component FAME mix (0.00004 mg/mL to 0.2 mg/mL). The injection volume was 1 μL.

The GC parameters were as follows: column oven temperature: 70° C., inject temperature: 230° C., inject mode: split, flow control mode: linear velocity, column flow: 1.0 mL/min, pressure: 53.5 mL/min, total flow: 29.0 mL/min, purge flow: 3.0 mL/min, split ratio: 25.0. The temperature gradient was as follows: 70° C. for 5 minutes, increasing to 350° C. at a rate of 5 degrees per minute, and then held at 350° C. for 1 minute. The MS parameters were as follows: ion source temperature: 200° C., interface temperature: 240° C., solvent cut time: 2 minutes, detector gain mode: relative, detector gain: 0.6 kV, threshold: 1000, group: 1, start time: 3 minutes, end time: 62 minutes, ACQ mode: scan, interval: 0.5 second, scan speed: 666 amu/sec., start M/z: 40, end M/z: 350. The instrument was tuned each time the column was cut or a new column was used.

The data were analyzed using the Shimadzu GC-MS Solutions software. Peak areas were integrated and exported to an Excel spreadsheet. Fatty acid peak areas were normalized to the internal standard, the amount of tissue weighed, and the slope of the corresponding calibration curve generated using the FAME mixture. Peak areas were also multiplied by the volume of hexane (0.75 mL) used to extract the fatty acids.

The same seed lines that were analyzed using GC-MS were also analyzed by FT-NIR spectroscopy, and the oil values determined by the GC-MS primary method were entered into the FT-NIR chemometrics software (Bruker Optics, Billerica, Mass.) to create a calibration curve for oil content. The actual oil content of each seed line analyzed using GC-MS was plotted on the x-axis of the calibration curve. The y-axis of the calibration curve represented the predicted values based on the best-fit line. Data points were continually added to the calibration curve data set.

T₂ seed from each transgenic plant line was analyzed by FT-NIR spectroscopy. Sarstedt tubes containing seeds were placed directly on the lamp, and spectra were acquired through the bottom of the tube. The spectra were analyzed to determine seed oil content using the FT-NIR chemometrics software (Bruker Optics) and the oil calibration curve. Results for experimental samples were compared to population means and standard deviations calculated for transgenic seed lines that were planted within 30 days of the lines being analyzed and grown under the same conditions. Typically, results from three to four events of each of 400 to 1600 different transgenic lines were used to calculate a population mean. Each data point was assigned a z-score (z=(x−mean)/std), and a p-value was calculated for the z-score.

Transgenic seed lines with protein levels in T₂ seed that differed by more than two standard deviations from the population mean were also analyzed to determine oil levels in the T₃ generation. Events of selected lines were planted in individual pots. The pots were arranged randomly in flats along with pots containing matched control plants in order to minimize microenvironment effects. Matched control plants contained an empty version of the vector used to generate the transgenic seed lines. T₃ seed from up to five plants from each event was collected and analyzed individually using FT-NIR spectroscopy. Data from replicate samples were averaged and compared to controls using the Student's t-test.

### Example 4

### Results for ME11370 Events

T₂ and T₃ seed from five events of ME11370 containing ANNOT ID 826303 was analyzed for total protein content using FT-NIR spectroscopy as described in Example 2.

The protein content in T₂ seed from five events of ME11370 was significantly increased compared to the mean protein content in seed from transgenic Arabidopsis lines planted within 30 days of ME11370. As presented in Table 1, the protein content was increased to 136%, 122%, 145%, 120%, and 159% in seed from events-01, -02, -03, -04 and -05, respectively, compared to the population mean.

TABLE 1
Protein content (% control) in T₂ and T₃ seed from
ME11370 events containing ANNOT ID 826303

| | Event -01 | Event -02 | Event -03 | Event -04 | Event -05 | Control |
|---|---|---|---|---|---|---|
| Protein content | 136 | 122 | 145 | 120 | 159 | 100 ± 19* |
| (% control) in T₂ seed | | | | | | |
| p-value | <0.01 | <0.01 | <0.01 | <0.01 | <0.01 | N/A |
| Protein content | 117 ± 4 | 130 ± 2 | 124 ± 5 | 122 | 118 ± 2 | 100 ± 4 |
| (% control) in T₃ seed | | | | | | |
| p-value | 0.01 | <0.01 | <0.01 | N/A | 0.01 | N/A |
| No. of T₂ plants | 4 | 4 | 4 | 2 | 3 | 20 |

*Population mean of the protein content in seed from transgenic lines planted within 30 days of ME11370. Variation is presented as the standard error of the mean.

The protein content in T₃ seed from four events of ME11370 was significantly increased compared to the protein content in corresponding control seed. As presented in Table 1, the protein content was increased to 117%, 130%, 124%, and 118% in seed from events-01, -02, -03 and -05, respectively, compared to the protein content in control seed.

T₂ and T₃ seed from five events of ME11370 containing ANNOT ID 826303 was also analyzed for total oil content using FT-NIR spectroscopy as described in Example 3.

The oil content in T₂ seed from ME11370 events was not observed to differ significantly from the mean oil content in seed from transgenic Arabidopsis lines planted within 30 days of ME11370 (Table 2).

TABLE 2
Oil content (% control) in T₂ and T₃ seed from
ME11370 events containing ANNOT ID 826303

| | Event -01 | Event -02 | Event -03 | Event -04 | Event -05 | Control |
|---|---|---|---|---|---|---|
| Oil content | 93 | 111 | 102 | 102 | 91 | 100 ± 9* |
| (% control) in T₂ seed | | | | | | |
| p-value | 0.26 | 0.45 | 0.48 | 0.49 | 0.44 | N/A |
| Oil content | 98 ± 2 | 98 ± 2 | 96 ± 1 | 97 ± 2 | 96 ± 3 | 100 ± 1 |
| (% control) in T₃ seed | | | | | | |
| p-value | 0.46 | 0.34 | 0.01 | 0.28 | 0.38 | N/A |
| No. of T₂ plants | 4 | 4 | 4 | 2 | 3 | 20 |

*Population mean of the oil content in seed from transgenic lines planted within 30 days of ME11370. Variation is presented as the standard error of the mean.

The oil content in T₃ seed from one event of ME11370 was significantly decreased compared to the oil content in corresponding control seed. As presented in Table 2, the oil content was decreased to 96% in seed from event-03 compared to the oil content in control seed.

The physical appearances of T₁ ME11370 plants were similar to those of corresponding control plants. There were no observable or statistically significant differences between T₂ plants from events-01, -03, and -05 of ME11370 and control plants in germination, onset of flowering, rosette area, fertility, and general morphology/architecture. The seed yield of T₂ plants from events-03 and -05 was comparable to that of control plants, while the seed yield of plants from event-01 was lower.

### Example 5

### Results for ME11410 Events

T₂ and T₃ seed from three events and two events, respectively, of ME11410 containing ANNOT ID 842015 was analyzed for total protein content using FT-NIR spectroscopy as described in Example 2.

The protein content in T₂ seed from three events of ME11410 was significantly increased compared to the mean protein content in seed from transgenic Arabidopsis lines planted within 30 days of ME11410. As presented in Table 3, the protein content was increased to 144%, 145%, and 136% in seed from events-02, -03, and -05, respectively, compared to the population mean.

TABLE 3
Protein content (% control) in T₂ and T₃ seed from
ME11410 events containing ANNOT ID 842015

| | Event -02 | Event -03 | Event -05 | Control |
|---|---|---|---|---|
| Protein content | 144 | 145 | 136 | 100 ± 18* |
| (% control) in T₂ seed | | | | |
| p-value | 0.01 | <0.01 | 0.01 | N/A |
| Protein content | 122 ± 4 | No data | 131 ± 5 | 100 ± 4 |
| (% control) in T₃ seed | | | | |
| p-value | 0.01 | No data | 0.02 | N/A |
| No. of T₂ plants | 4 | No data | 3 | 15 |

*Population mean of the protein content in seed from transgenic lines planted within 30 days of ME11410. Variation is presented as the standard error of the mean.

The protein content in T₃ seed from two events of ME11410 was significantly increased compared to the protein content in corresponding control seed. As presented in Table 3, the protein content was increased to 122% and 131% in seed from events-02 and -05, respectively, compared to the protein content in control seed.

T₂ and T₃ seed from three events of ME11410 containing ANNOT ID 842015 was also analyzed for total oil content using FT-NIR spectroscopy as described in Example 3.

The oil content in T₂ seed from ME11410 events was not observed to differ significantly from the mean oil content in seed from transgenic Arabidopsis lines planted within 30 days of ME11410 (Table 4).

TABLE 4
Oil content (% control) in T₂ and T₃ seed from
ME11410 events containing ANNOT ID 842015

| | Event -02 | Event -03 | Event -05 | Control |
|---|---|---|---|---|
| Oil content | 102 | 104 | 105 | 100 ± 9* |
| (% control) in T₂ seed | | | | |
| p-value | 0.57 | 0.46 | 0.43 | N/A |
| Oil content | 96 ± 1 | No data | 98 ± 1 | 100 ± 1 |
| (% control) in T₃ seed | | | | |
| p-value | 0.01 | No data | 0.21 | N/A |
| No. of T₂ plants | 4 | No data | 3 | 20 |

*Population mean of the oil content in seed from transgenic lines planted within 30 days of ME11410. Variation is presented as the standard error of the mean.

The oil content in T₃ seed from one event of ME11410 was significantly decreased compared to the oil content in corresponding control seed. As presented in Table 4, the oil content was decreased to 96% in seed from event-02 compared to the oil content in control seed.

The physical appearances of T₁ ME11410 plants were similar to those of corresponding control plants. There were no observable or statistically significant differences between T₃ ME11410 and control plants in germination, onset of flowering, rosette area, fertility, general morphology/architecture, and seed yield.

### Example 6

### Results for ME02482 Events

T₂ and T₃ seed from five events of ME02482 containing CLONE ID 97982 was analyzed for total protein content using FT-NIR spectroscopy as described in Example 2.

The protein content in T₂ seed from four events of ME02482 was significantly increased compared to the mean protein content of seed from transgenic Arabidopsis lines planted within 30 days of ME02482. As presented in Table 5, the protein content was increased to 130% in seed from events-12 and -13, and to 128% and 135% in seed from events-16 and -19, respectively, compared to the population mean.

TABLE 5
Protein content (% control) in T₂ and T₃ seed
from ME02482 events containing CLONE ID 97982

| | Event -12 | Event -13 | Event -14 | Event -16 | Event -19 | Control |
|---|---|---|---|---|---|---|
| Protein content | 130 | 130 | 129 | 128 | 135 | 100 ± 13* |
| (% control) in T₂ seed | | | | | | |
| p-value | 0.049 | 0.047 | 0.06 | 0.04 | 0.02 | N/A |
| Protein content | 101 ± 1 | 104 ± 3 | 103 ± 5 | 109 ± 3 | 107 ± 2 | 100 ± 1 |
| (% control) in T₃ seed | | | | | | |
| p-value | 0.21 | 0.12 | 0.40 | 0.04 | 0.01 | N/A |
| No. of T₂ plants | 5 | 4 | 4 | 5 | 5 | 15 |

*Population mean of the protein content in seed from transgenic lines planted within 30 days of ME02482. Variation is presented as the standard error of the mean.

The protein content in T₃ seed from two events of ME02482 was significantly increased compared to the protein content in corresponding control seed. As presented in Table 5, the protein content was increased to 109% and 107% in seed from events-16 and -19, respectively.

T₂ and T₃ seed from five events of ME02482 containing CLONE ID 97982 was also analyzed for total oil content using FT-NIR spectroscopy as described in Example 3. The oil content in T₂ seed from ME02482 events was not observed to differ significantly from the mean oil content in seed from transgenic Arabidopsis lines planted within 30 days of ME02482 (Table 6).

TABLE 6
Oil content (% control) in T₂ and T₃ seed from
ME02482 events containing CLONE ID 97982

| | Event -12 | Event -13 | Event -14 | Event -16 | Event -19 | Control |
|---|---|---|---|---|---|---|
| Oil content | 93 | 98 | 95 | 104 | 99 | 100 ± 1* |
| (% control) in T₂ seed | | | | | | |
| p-value | 0.15 | 0.42 | 0.24 | 0.21 | 0.48 | N/A |
| Oil content | 92 ± 1 | 95 ± 1 | 92 ± 5 | 91 ± 2 | 93 ± 1 | 100 ± 2 |
| (% control) in T₃ seed | | | | | | |
| p-value | <0.01 | <0.01 | 0.04 | <0.01 | <0.01 | N/A |
| No. of T₂ plants | 5 | 4 | 4 | 5 | 5 | 15 |

*Population mean of the oil content in seed from transgenic lines planted within 30 days of ME02482. Variation is presented as the standard error of the mean.

The oil content in T₃ seed from five events of ME02482 was significantly decreased compared to the oil content in corresponding control seed. As presented in Table 6, the oil content was decreased to 92% in seed from events-12 and -14, and to 95%, 91%, and 93% in seed from events-13, -16, and -19, respectively, compared to the oil content in corresponding control seed.

The physical appearances of T₁ ME02482 plants were similar to those of corresponding control plants. There were no observable or statistically significant differences between T₂ plants from events-16 and -19 of ME02482 and control plants in germination, onset of flowering, rosette area, fertility, general morphology/architecture, or seed yield.

### Example 7

### Results for ME11409 Events

T₂ and T₃ seed from four events of ME11409 containing ANNOT ID 571199 was analyzed for total protein content using FT-NIR spectroscopy as described in Example 2.

The protein content in T₂ seed from three events of ME11409 was significantly increased compared to the mean protein content in seed from transgenic Arabidopsis lines planted within 30 days of ME11409. As presented in Table 7, the protein content was increased to 134%, 133%, and 127% in seed from events-03, -04, and -05, respectively, compared to the population mean.

TABLE 7
Protein content (% control) in T₂ and T₃ seed
from ME11409 events containing ANNOT ID 571199

| | Event -01 | Event -03 | Event -04 | Event -05 | Control |
|---|---|---|---|---|---|
| Protein content | 125 | 134 | 133 | 127 | 100 ± 18* |
| (% control) in T₂ seed | | | | | |
| p-value | 0.11 | 0.02 | 0.03 | 0.04 | N/A |
| Protein content | 126 | 122 | 124 ± 1 | 136 ± 4 | 100 ± 4 |
| (% control) in T₃ seed | | | | | |
| p-value | N/A | N/A | <0.01 | 0.01 | N/A |
| No. of T₂ plants | 2 | 2 | 5 | 3 | 15 |

*Population mean of the protein content in seed from transgenic lines planted within 30 days of ME11409. Variation is presented as the standard error of the mean.

The protein content in T₃ seed from four events of ME was increased compared to the protein content in corresponding control seed. As presented in Table 7, the protein content was increased to 126%, 122%, 124%, and 136% in seed from events-01, -03, -04 and -05, respectively, compared to the protein content in control seed.

T₂ and T₃ seed from four events of ME11409 containing ANNOT ID 571199 was also analyzed for total oil content using FT-NIR spectroscopy as described in Example 3. The oil content in T₂ seed from ME11409 events was not observed to differ significantly from the mean oil content in seed from transgenic Arabidopsis lines planted within 30 days of ME11409 (Table 8).

TABLE 8
Oil content (% control) in T₂ and T₃ seed from
ME11409 events containing ANNOT ID 571199

| | Event -01 | Event -03 | Event -04 | Event -05 | Control |
|---|---|---|---|---|---|
| Oil content | 101 | 102 | 104 | 105 | 100 ± 9* |
| (% control) in T₂ seed | | | | | |
| p-value | 0.66 | 0.57 | 0.46 | 0.43 | N/A |
| Oil content | 87 | 97 | 90 ± 1 | 113 ± 3 | 100 ± 1 |
| (% control) in T₃ seed | | | | | |
| p-value | N/A | N/A | <0.01 | 0.03 | N/A |
| No. of T₂ plants | 2 | 2 | 5 | 3 | 20 |

*Population mean of the oil content in seed from transgenic lines planted within 30 days of ME11409. Variation is presented as the standard error of the mean.

The oil content in T₃ seed from three events of ME11409 was decreased compared to the oil content in corresponding control seed. As presented in Table 8, the oil content was decreased to 87%, 97%, and 90% in seed from events-01, -03, and -04, respectively, compared to the oil content in control seed. The oil content in T₃ seed from one event of ME11409 was significantly increased compared to the oil content in corresponding control seed. As presented in Table 8, the oil content was increased to 113% in T₃ seed from event-05 compared to the oil content in control seed.

The physical appearances of T₁ ME11409 plants were similar to those of corresponding control plants. There were no observable or statistically significant differences between T₃ ME11409 and control plants in germination, onset of flowering, rosette area, fertility, or general morphology/architecture. The seed yield of plants from event-04 was comparable to that of control plants, while the seed yield of plants from event-05 was lower.

### Example 8

### Results for ME10870 Events

T₂ and T₃ seed from three events of ME10870 containing ANNOT ID 564367 was analyzed for total protein content using FT-NIR spectroscopy as described in Example 2.

The protein content in T₂ seed from three events of ME10870 was significantly increased compared to the mean protein content in seed from transgenic Arabidopsis lines planted within 30 days of ME10870. As presented in Table 9, the protein content was increased to 141%, 134%, and 154% in seed from events-02, -03, and -04, respectively, compared to the population mean.

TABLE 9
Protein content (% control) in T₂ and T₃ seed
from ME10870 events containing ANNOT ID 564367

| | Event -02 | Event -03 | Event -04 | Control |
|---|---|---|---|---|
| Protein content | 141 | 134 | 154 | 100 ± 19* |
| (% control) in T₂ seed | | | | |
| p-value | 0.02 | 0.04 | 0.01 | N/A |
| Protein content | 108 ± 1 | 106 ± 1 | 100 ± 2 | 100 ± 1 |
| (% control) in T₃ seed | | | | |
| p-value | <0.01 | <0.01 | 0.91 | N/A |
| No. of T₂ plants | 5 | 5 | 3 | 15 |

*Population mean of the protein content in seed from transgenic lines planted within 30 days of ME10870. Variation is presented as the standard error of the mean.

The protein content in T₃ seed from two events of ME10870 was significantly increased compared to the protein content in corresponding control seed. As presented in Table 9, the protein content was increased to 108% and 106% in seed from events-02 and -03, respectively, compared to the protein content in control seed.

T₂ and T₃ seed from three events of ME10870 containing ANNOT ID 564367 was also analyzed for total oil content using FT-NIR spectroscopy as described in Example 3.

The oil content in T₂ seed from one event of ME10870 was significantly decreased compared to the mean oil content in seed from transgenic Arabidopsis lines planted within 30 days of ME10870. As presented in Table 10, the oil content was decreased to 74% in seed from event-04 compared to the population mean.

TABLE 10
Oil content (% control) in T₂ and T₃ seed from
ME10870 events containing ANNOT ID 564367

| | Event -02 | Event -03 | Event -04 | Control |
|---|---|---|---|---|
| Oil content | 82 | 94 | 74 | 100 ± 9* |
| (% control) in T₂ seed | | | | |
| p-value | 0.12 | 0.49 | 0.03 | N/A |
| Oil content | 97 ± 1 | 96 ± 4 | 96 ± 2 | 100 ± 2 |
| (% control) in T₃ seed | | | | |
| p-value | 0.09 | 0.38 | 0.15 | N/A |
| No. of T₂ plants | 5 | 5 | 3 | 15 |

*Population mean of the oil content in seed from transgenic lines planted within 30 days of ME10870. Variation is presented as the standard error of the mean.

The oil content in T₃ seed from ME10870 events was not observed to differ significantly from the oil content in control seed (Table 10).

The physical appearances of T₁ ME10870 plants were similar to those of corresponding control plants. There were no observable or statistically significant differences between T₃ ME10870 and control plants in germination, onset of flowering, rosette area, fertility, general morphology/architecture, or seed yield.

### Example 9

### Results for ME11351 Events

T₂ and T₃ seed from four events of ME11351 containing ANNOT ID 851745 was analyzed for total protein content using FT-NIR spectroscopy as described in Example 2.

The protein content in T₂ seed from three events of ME11351 was significantly increased compared to the mean protein content in seed from transgenic Arabidopsis lines planted within 30 days of ME11351. As presented in Table 11, the protein content was increased to 147%, 141%, and 134% in seed from events-02, -03, and -05, respectively, compared to the population mean.

TABLE 11
Protein content (% control) in T₂ and T₃ seed
from ME11351 events containing ANNOT ID 851745

| | Event -01 | Event -02 | Event -03 | Event -05 | Control |
|---|---|---|---|---|---|
| Protein content | 132 | 147 | 141 | 134 | 100 ± 19* |
| (% control) in T₂ seed | | | | | |
| p-value | 0.138 | 0.02 | 0.045 | 0.03 | N/A |
| Protein content | 103 ± 4 | 109 ± 1 | 104 | 106 ± 1 | 100 ± 1 |
| (% control) in T₃ seed | | | | | |
| p-value | 0.54 | <0.01 | N/A | <0.01 | N/A |
| No. of T₂ plants | 3 | 5 | 1 | 5 | 15 |

*Population mean of the protein content in seed from transgenic lines planted within 30 days of ME11351. Variation is presented as the standard error of the mean.

The protein content in T₃ seed from two events of ME11351 was significantly increased compared to the protein content in corresponding control seed. As presented in Table 11, the protein content was increased to 109% and 106% in seed from events-02 and -05, respectively, compared to the protein content in control seed.

T₂ and T₃ seed from four events of ME11351 containing ANNOT ID 851745 was also analyzed for total oil content using FT-NIR spectroscopy as described in Example 3. The oil content in T₂ and T₃ seed from ME11351 events was not observed to differ significantly from the oil content in corresponding control seed (Table 12).

TABLE 12
Oil content (% control) in T₂ and T₃ seed from
ME11351 events containing ANNOT ID 851745

| | Event -01 | Event -02 | Event -03 | Event -05 | Control |
|---|---|---|---|---|---|
| Oil content | 95 | 101 | 105 | 104 | 100 ± 9* |
| (% control) in T₂ seed | | | | | |
| p-value | 0.16 | 0.33 | 0.48 | 0.44 | N/A |
| Oil content | 100 ± 1 | 97 ± 2 | 101 | 98 ± 2 | 100 ± 2 |
| (% control) in T₃ seed | | | | | |
| p-value | 0.96 | 0.11 | N/A | 0.38 | N/A |
| No. of T₂ plants | 3 | 5 | 1 | 5 | 15 |

*Population mean of the oil content in seed from transgenic lines planted within 30 days of ME11351. Variation is presented as the standard error of the mean.

The physical appearances of T₁ ME11351 plants were similar to those of corresponding control plants. There were no observable or statistically significant differences between T₃ ME11351 and control plants in germination, onset of flowering, rosette area, fertility, general morphology/architecture, or seed yield.

### Example 10

### Results for ME07978 Events

T₂ and T₃ seed from five events of ME07978 containing 3′-truncated CLONE ID 258034 was analyzed for total protein content using FT-NIR spectroscopy as described in Example 2.

The protein content in T₂ seed from five events of ME07978 was significantly increased compared to the mean protein content in seed from transgenic Arabidopsis lines planted within 30 days of ME07978. As presented in Table 13, the protein content was increased to 127%, 132%, 129%, 134%, and 125% in seed from events-01, -02, -03, -04, and -05, respectively, compared to the population mean.

TABLE 13
Protein content (% control) in T₂ and T₃ seed from ME07978
events containing 3′ truncated CLONE ID 258034

| | Event -01 | Event -02 | Event -03 | Event -04 | Event -05 | Control |
|---|---|---|---|---|---|---|
| Protein content | 127 | 132 | 129 | 134 | 125 | 100 ± 15* |
| (% control) in T₂ seed | | | | | | |
| p-value | 0.04 | 0.02 | 0.03 | 0.01 | 0.05 | N/A |
| Protein content | 97 ± 1 | 107 ± 2 | 101 ± 3 | 111 ± 0 | 107 ± 4 | 100 ± 4 |
| (% control) in T₃ seed | | | | | | |
| p-value | 0.03 | 0.01 | 0.76 | <0.01 | 0.26 | N/A |

*Population mean of the protein content in seed from transgenic lines planted within 30 days of ME07978. Variation is presented as the standard error of the mean.

The protein content in T₃ seed from two events of ME07978 was significantly increased compared to the protein content in corresponding control seed. As presented in Table 13, the protein content was increased to 107% and 111% in seed from events-02 and -04, respectively, compared to the protein content in control seed. The protein content in T₃ seed from one event of ME07978 was significantly decreased compared to the protein content in corresponding control seed. As presented in Table 13, the protein content was decreased to 97% in seed from event-01 compared to the protein content in control seed.

### Example 11

### Determination of Functional Homolog and/or Ortholog Sequences

A subject sequence was considered a functional homolog or ortholog of a query sequence if the subject and query sequences encoded proteins having a similar function and/or activity. A process known as Reciprocal BLAST (Rivera et al., Proc. Natl. Acad. Sci. USA, 95:6239-6244 (1998)) was used to identify potential functional homolog and/or ortholog sequences from databases consisting of all available public and proprietary peptide sequences, including NR from NCBI and peptide translations from Ceres clones.

Before starting a Reciprocal BLAST process, a specific query polypeptide was searched against all peptides from its source species using BLAST in order to identify polypeptides having BLAST sequence identity of 80% or greater to the query polypeptide and an alignment length of 85% or greater along the shorter sequence in the alignment. The query polypeptide and any of the aforementioned identified polypeptides were designated as a cluster.

The BLASTP version 2.0 program from Washington University at Saint Louis, Mo., USA was used to determine BLAST sequence identity and E-value. The BLASTP version 2.0 program includes the following parameters: 1) an E-value cutoff of 1.0e-5; 2) a word size of 5; and 3) the -postsw option. The BLAST sequence identity was calculated based on the alignment of the first BLAST HSP (High-scoring Segment Pairs) of the identified potential functional homolog and/or ortholog sequence with a specific query polypeptide. The number of identically matched residues in the BLAST HSP alignment was divided by the HSP length, and then multiplied by 100 to get the BLAST sequence identity. The HSP length typically included gaps in the alignment, but in some cases gaps were excluded.

The main Reciprocal BLAST process consists of two rounds of BLAST searches; forward search and reverse search. In the forward search step, a query polypeptide sequence, “polypeptide A,” from source species SA was BLASTed against all protein sequences from a species of interest. Top hits were determined using an E-value cutoff of 10⁻⁵ and a sequence identity cutoff of 35%. Among the top hits, the sequence having the lowest E-value was designated as the best hit, and considered a potential functional homolog or ortholog. Any other top hit that had a sequence identity of 80% or greater to the best hit or to the original query polypeptide was considered a potential functional homolog or ortholog as well. This process was repeated for all species of interest.

In the reverse search round, the top hits identified in the forward search from all species were BLASTed against all protein sequences from the source species SA. A top hit from the forward search that returned a polypeptide from the aforementioned cluster as its best hit was also considered as a potential functional homolog or ortholog.

Functional homologs and/or orthologs were identified by manual inspection of potential functional homolog and/or ortholog sequences. Representative functional homologs and/or orthologs for SEQ ID NO:96, SEQ ID NO:102, SEQ ID NO:118, and SEQ ID NO:128 are shown in FIGS. 1-4, respectively. The BLAST percent identities and E-values of functional homologs and/or orthologs to SEQ ID NO:96, SEQ ID NO:102, SEQ ID NO:118, and SEQ ID NO:128 are shown below in Tables 14-17, respectively. The BLAST sequence identities and E-values given in Tables 14-17 were taken from the forward search round of the Reciprocal BLAST process.

TABLE 14
Percent identity to ANNOT ID 826303 (SEQ ID NO: 96)

| | | SEQ | % | |
|---|---|---|---|---|
| Designation | Species | ID NO: | Identity | e-value |
| Ceres CLONE | Glycine max | 97 | 58.5 | 3.10E−75 |
| ID no. 1103899 | | | | |
| Ceres CLONE | Zea mays | 98 | 56.1 | 8.90E−69 |
| ID no. 463034 | | | | |
| Ceres CLONE | Panicum virgatum | 100 | 52 | 2.69E−67 |
| ID no. 1816436 | | | | |

TABLE 15
Percent identity to ANNOT ID 571199 (SEQ ID NO: 102)

| | | SEQ | % | |
|---|---|---|---|---|
| Designation | Species | ID NO: | Identity | e-value |
| Public GI | Arabidopsis thaliana | 103 | 99.7 | 9.79E−182 |
| no. 110737799 | | | | |
| Ceres ANNOT | Populus balsamifera | 106 | 68.1 | 7.20E−115 |
| ID no. 1469254 | subsp. trichocarpa | | | |
| Public GI | Medicago truncatula | 107 | 67 | 2.09E−108 |
| no. 92895842 | | | | |
| Ceres CLONE | Glycine max | 108 | 66.1 | 4.19E−110 |
| ID no. 1121764 | | | | |
| Public GI | Oryza sativa subsp. | 109 | 65.1 | 1.00E−90 |
| no. 33146851 | japonica | | | |
| Ceres CLONE | Zea mays | 110 | 60.9 | 3.10E−98 |
| ID no. 278526 | | | | |

TABLE 16
Percent identity to ANNOT ID 564367 (SEQ ID NO: 118)

| | | SEQ | % | |
|---|---|---|---|---|
| Designation | Species | ID NO: | Identity | e-value |
| Ceres CLONE | Glycine max | 119 | 56.4 | 9.79E−47 |
| ID no. 594825 | | | | |
| Ceres ANNOT | Populus balsamifera | 122 | 53.8 | 4.19E−46 |
| ID no. 1539862 | subsp. trichocarpa | | | |
| Ceres ANNOT | Populus balsamifera | 125 | 53.8 | 4.19E−46 |
| ID no. 1486448 | subsp. trichocarpa | | | |
| Public GI | Oryza sativa subsp. | 126 | 50.9 | 3.50E−35 |
| no. 115479583 | japonica | | | |

TABLE 17
Percent identity to ANNOT ID 851745 (SEQ ID NO: 128)

| | | SEQ | % | |
|---|---|---|---|---|
| Designation | Species | ID NO: | Identity | e-value |
| Ceres ANNOT | Populus balsamifera | 131 | 56.1 | 2.20E−81 |
| ID no. 1455259 | subsp. trichocarpa | | | |
| Ceres CLONE | Gossypium hirsutum | 133 | 55 | 1.99E−78 |
| ID no. 1939499 | | | | |
| Ceres CLONE | Glycine max | 134 | 53.7 | 4.79E−77 |
| ID no. 605517 | | | | |

### Other Embodiments

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

1.-44. (canceled)

45. A plant cell comprising an exogenous nucleic acid comprising a nucleotide sequence encoding a polypeptide having 80 percent or greater sequence identity to an amino acid sequence selected from the group consisting of SEQ ID NOs:96-98, SEQ ID NO:100, SEQ ID NOs:102-103, SEQ ID NOs:106-110, SEQ ID NO:112, SEQ ID NO:114, SEQ ID NO:116, SEQ ID NOs:118-119, SEQ ID NO:122, SEQ ID NOs:125-126, SEQ ID NO:128, SEQ ID NO:131, SEQ ID NOs:133-134, SEQ ID NO:136, SEQ ID NO:138, SEQ ID NOs:140-141, SEQ ID NOs:143-146, SEQ ID NO:148, SEQ ID NO:150, SEQ ID NO:152, SEQ ID NO:154, SEQ ID NOs:156-157, SEQ ID NO:159, SEQ ID NO:161, SEQ ID NO:163, SEQ ID NO:165, SEQ ID NOs:167-175, SEQ ID NO:177, SEQ ID NOs:179-182, SEQ ID NO:184, SEQ ID NO:186, SEQ ID NO:186, SEQ ID NOs:188-189, SEQ ID NO:191, SEQ ID NOs:193-205, SEQ ID NOs:207-210, SEQ ID NOs:212-220, SEQ ID NO:222, SEQ ID NO:224, SEQ ID NO:226, SEQ ID NO:228, SEQ ID NO:230, SEQ ID NO:232, SEQ ID NO:234, SEQ ID NO:236, SEQ ID NOs:238-239, SEQ ID NO:241, and SEQ ID NOs:243-247, wherein a tissue of a plant produced from said plant cell has a difference in the level of protein as compared to the corresponding level in tissue of a control plant that does not comprise said nucleic acid.

46. A plant cell comprising an exogenous nucleic acid comprising a nucleotide sequence encoding a polypeptide having 80 percent or greater sequence identity to an amino acid sequence selected from the group consisting of SEQ ID NOs:96-98, SEQ ID NO:100, SEQ ID NO:102, SEQ ID NO:106, SEQ ID NO:108, SEQ ID NO:110, SEQ ID NO:112, SEQ ID NO:114, SEQ ID NO:116, SEQ ID NOs:118-119, SEQ ID NO:122, SEQ ID NO:125, SEQ ID NO:128, SEQ ID NO:131, SEQ ID NOs:133-134, SEQ ID NO:136, SEQ ID NO:138, SEQ ID NO:140, SEQ ID NO:143, SEQ ID NO:148, SEQ ID NO:150, SEQ ID NO:152, SEQ ID NO:154, SEQ ID NO:156, SEQ ID NO:159, SEQ ID NO:161, SEQ ID NO:163, SEQ ID NO:165, SEQ ID NO:167, SEQ ID NO:177, SEQ ID NO:179, SEQ ID NO:181, SEQ ID NO:184, SEQ ID NO:186, SEQ ID NO:188, SEQ ID NO:191, SEQ ID NO:193, SEQ ID NO:207, SEQ ID NO:212, SEQ ID NO:222, SEQ ID NO:224, SEQ ID NO:226, SEQ ID NO:228, SEQ ID NO:230, SEQ ID NO:232, SEQ ID NO:234, SEQ ID NO:236, SEQ ID NO:238, SEQ ID NO:241, and SEQ ID NO:243, wherein a tissue of a plant produced from said plant cell has a difference in the level of protein as compared to the corresponding level in tissue of a control plant that does not comprise said nucleic acid.

47. A plant cell comprising an exogenous nucleic acid comprising a nucleotide sequence having 80 percent or greater sequence identity to a nucleotide sequence corresponding to SEQ ID NO:115, wherein a tissue of a plant produced from said plant cell has a difference in the level of protein as compared to the corresponding level in tissue of a control plant that does not comprise said nucleic acid.

48. A plant cell comprising an exogenous nucleic acid comprising a nucleotide sequence having 80 percent or greater sequence identity to a nucleotide sequence selected from the group consisting of SEQ ID NO:95, SEQ ID NO:99, SEQ ID NO:101, SEQ ID NO:104, SEQ ID NO:105, SEQ ID NO:111, SEQ ID NO:113, SEQ ID NO:115, SEQ ID NO:117, SEQ ID NO:120, SEQ ID NO:121, SEQ ID NO:123, SEQ ID NO:124, SEQ ID NO:127, SEQ ID NO:129, SEQ ID NO:130, SEQ ID NO:132, SEQ ID NO:135, SEQ ID NO:137, SEQ ID NO:139, SEQ ID NO:142, SEQ ID NO:147, SEQ ID NO:149, SEQ ID NO:151, SEQ ID NO:153, SEQ ID NO:155, SEQ ID NO:158, SEQ ID NO:160, SEQ ID NO:162, SEQ ID NO:164, SEQ ID NO:166, SEQ ID NO:176, SEQ ID NO:178, SEQ ID NO:183, SEQ ID NO:187, SEQ ID NO:190, SEQ ID NO:192, SEQ ID NO:206, SEQ ID NO:211, SEQ ID NO:221, SEQ ID NO:223, SEQ ID NO:225, SEQ ID NO:227, SEQ ID NO:229, SEQ ID NO:231, SEQ ID NO:233, SEQ ID NO:235, SEQ ID NO:237, SEQ ID NO:240, SEQ ID NO:242, and SEQ ID NO:248, wherein a tissue of a plant produced from said plant cell has a difference in the level of protein as compared to the corresponding level in tissue of a control plant that does not comprise said nucleic acid.

49. The plant cell of claim 45, wherein said sequence identity is 85 percent or greater.

50. The plant cell of claim 49, wherein said sequence identity is 90 percent or greater.

51. The plant cell of claim 50, wherein said sequence identity is 95 percent or greater.

52. The plant cell of claim 46, wherein said nucleotide sequence encodes a polypeptide comprising an amino acid sequence corresponding to SEQ ID NO:96, SEQ ID NO:102, SEQ ID NO:112, SEQ ID NO:114, SEQ ID NO:116, SEQ ID NO:118, SEQ ID NO:128, or SEQ ID NO:181.

53. The plant cell of claim 47, wherein said exogenous nucleic acid comprises a nucleotide sequence corresponding to SEQ ID NO:115.

54. The plant cell of claim 45, wherein said difference is an increase in the level of protein.

55. The plant cell of claim 45, wherein said exogenous nucleic acid is operably linked to a regulatory region.

56. The plant cell of claim 55, wherein said regulatory region is a promoter.

57. The plant cell of claim 56, wherein said promoter is a tissue-preferential, broadly expressing, or inducible promoter.

58. The plant cell of claim 57, wherein said promoter is a maturing endosperm promoter.

59. The plant cell of claim 45, wherein said plant is a dicot.

60. The plant cell of claim 59, wherein said plant is a member of the genus Arachis, Brassica, Carthamus, Glycine, Gossypium, Helianthus, Lactuca, Linum, Lycopersicon, Medicago, Olea, Pisum, Solanum, Trifolium, or Vitis.

61. The plant cell of claim 45, wherein said plant is a monocot.

62. The plant cell of claim 61, wherein said plant is a member of the genus Avena, Elaeis, Hordeum, Musa, Oryza, Phleum, Secale, Sorghum, Triticosecale, Triticum, or Zea.

63. The plant cell of claim 52, wherein said plant is a member of the genus Oryza.

64. The plant cell of claim 45, wherein said tissue is seed tissue.

65. A transgenic plant comprising the plant cell of claim 45.

66. Progeny of the plant of claim 65, wherein said progeny has a difference in the level of protein as compared to the level of protein in a corresponding control plant that does not comprise said nucleic acid.

67. Seed from a transgenic plant according to claim 66.

68. Vegetative tissue from a transgenic plant according to claim 65.

69. A food product comprising seed or vegetative tissue from a transgenic plant according to claim 65.

70. A feed product comprising seed or vegetative tissue from a transgenic plant according to claim 65.

71. An isolated nucleic acid comprising a nucleotide sequence having 95% or greater sequence identity to the nucleotide sequence set forth in SEQ ID NO:105, SEQ ID NO:121, SEQ ID NO:124, SEQ ID NO:130, or SEQ ID NO:132.

72. An isolated nucleic acid comprising a nucleotide sequence encoding a polypeptide having 80% or greater sequence identity to the amino acid sequence set forth in SEQ ID NO:106, SEQ ID NO:122, SEQ ID NO:125, SEQ ID NO:131, or SEQ ID NO:133.

73.-76. (canceled)
